SEQUENCE LISTING (144 sequences)

SEQ ID NO: 1 - protein, 12 aa, Artificial Sequence
  A peptide containing D amino acids.
QXQXQXQXQXQX

SEQ ID NO: 2 - protein, 8 aa, Artificial Sequence
  A peptide containing D amino acids.
QXWXWXWX

SEQ ID NO: 3 - protein, 8 aa, Artificial Sequence
  A peptide containing D amino acids.
KXWXWXWX

SEQ ID NO: 4 - protein, 8 aa, Artificial Sequence
  A peptide containing D amino acids.
KXKXKXKX

SEQ ID NO: 5 - protein, 8 aa, Artificial Sequence
  A peptide containing D amino acids.
XKXWXWXW

SEQ ID NO: 6 - protein, 8 aa, Artificial Sequence
  A peptide containing D amino acids.
XHXWXWXW

SEQ ID NO: 7 - protein, 8 aa, Artificial Sequence
  A peptide containing D amino acids.
XGXWXWXW

SEQ ID NO: 8 - protein, 8 aa, Artificial Sequence
  A peptide containing D amino acids.
XQXWXWXW

SEQ ID NO: 9 - protein, 8 aa, Artificial Sequence
  A peptide containing D amino acids.
KXRXLXLX

SEQ ID NO: 10 - protein, 8 aa, Artificial Sequence
  A peptide containing D amino acids.
XQXWXWXW

SEQ ID NO: 11 - protein, 8 aa, Artificial Sequence
  A peptide containing D amino acids.
XQXWXWXW

SEQ ID NO: 12 - protein, 8 aa, Artificial Sequence
  A peptide containing D amino acids.
XSXWXWXW

SEQ ID NO: 13 - protein, 8 aa, Artificial Sequence
  A peptide containing D amino acids.
XHXWXWXW

SEQ ID NO: 14 - protein, 8 aa, Artificial Sequence
  A peptide containing D amino acids.
XKXWXWXW

SEQ ID NO: 15 - protein, 8 aa, Artificial Sequence
  A peptide containing D amino acids.
XHXWXWXW

SEQ ID NO: 16 - protein, 8 aa, Artificial Sequence
  A peptide containing D amino acids.
XKXWXWXW

SEQ ID NO: 17 - protein, 8 aa, Artificial Sequence
  A peptide containing D amino acids.
XKXWXWXW

SEQ ID NO: 18 - protein, 8 aa, Artificial Sequence
  A peptide containing D amino acids.
XRXWXWXW

SEQ ID NO: 19 - protein, 8 aa, Artificial Sequence
  A peptide containing D amino acids.
XRXWXWXW

SEQ ID NO: 20 - protein, 8 aa, Artificial Sequence
  A peptide containing D amino acids.
XRXWXWXW

SEQ ID NO: 21 - protein, 8 aa, Artificial Sequence
  A peptide containing D amino acids.
XKXWXWXW

SEQ ID NO: 22 - protein, 8 aa, Artificial Sequence
  A peptide containing D amino acids.
XHXWXWXW

SEQ ID NO: 23 - protein, 9 aa, Artificial Sequence
  A peptide containing D amino acids.
WXKXWXWXW

SEQ ID NO: 24 - protein, 8 aa, Artificial Sequence
  A peptide containing D amino acids.
XKXWXWXW

SEQ ID NO: 25 - protein, 8 aa, Artificial Sequence
  A peptide containing D amino acids.
XKXWXWXX

SEQ ID NO: 26 - protein, 6 aa, Artificial Sequence
  A peptide containing D amino acids.
XKXWXW

SEQ ID NO: 27 - protein, 6 aa, Artificial Sequence
  A peptide containing D amino acids.
XHXWXW

SEQ ID NO: 28 - protein, 6 aa, Artificial Sequence
  A peptide containing D amino acids.
XSXWXW

SEQ ID NO: 29 - protein, 6 aa, Artificial Sequence
  A peptide containing D amino acids.
XRXWXW

SEQ ID NO: 30 - protein, 8 aa, Artificial Sequence
  A peptide containing D amino acids.
QXXXXXWX

SEQ ID NO: 31 - protein, 8 aa, Artificial Sequence
  A peptide containing D amino acids.
QXXXXXWX

SEQ ID NO: 32 - protein, 8 aa, Artificial Sequence
  A peptide containing D amino acids.
QXXXXXWX

SEQ ID NO: 33 - protein, 8 aa, Artificial Sequence
  A peptide containing D amino acids.
QXXXXXWX

SEQ ID NO: 34 - protein, 8 aa, Artificial Sequence
  A peptide containing D amino acids.
QXXXFXWX

SEQ ID NO: 35 - protein, 8 aa, Artificial Sequence
  A peptide containing D amino acids.
QXXXLXWX

SEQ ID NO: 36 - protein, 8 aa, Artificial Sequence
  A peptide containing D amino acids.
QXWXLXWX

SEQ ID NO: 37 - protein, 8 aa, Artificial Sequence
  A peptide containing D amino acids.
QXXXWXWX

SEQ ID NO: 38 - protein, 8 aa, Artificial Sequence
  A peptide containing D amino acids.
QXVXLXWX

SEQ ID NO: 39 - protein, 8 aa, Artificial Sequence
  A peptide containing D amino acids.
QXFXLXWX

SEQ ID NO: 40 - protein, 8 aa, Artificial Sequence
  A peptide containing D amino acids.
KXWXWXWX

SEQ ID NO: 41 - protein, 8 aa, Artificial Sequence
  A peptide containing D amino acids.
KXWXWXWX

SEQ ID NO: 42 - protein, 8 aa, Artificial Sequence
  A peptide containing D amino acids.
KXWXWXWX

SEQ ID NO: 43 - protein, 8 aa, Artificial Sequence
  A peptide containing D amino acids.
KXWXWXWX

SEQ ID NO: 44 - protein, 8 aa, Artificial Sequence
  A peptide containing D amino acids.
KXWXWXKX

SEQ ID NO: 45 - protein, 8 aa, Artificial Sequence
  A peptide containing D amino acids.
KXKXAXAX

SEQ ID NO: 46 - protein, 8 aa, Artificial Sequence
  A peptide containing D amino acids.
KXKXAXWX

SEQ ID NO: 47 - protein, 8 aa, Artificial Sequence
  A peptide containing D amino acids.
KXKXFXAX

SEQ ID NO: 48 - protein, 8 aa, Artificial Sequence
  A peptide containing D amino acids.
KXKXFXFX

SEQ ID NO: 49 - protein, 8 aa, Artificial Sequence
  A peptide containing D amino acids.
KXKXFXWX

SEQ ID NO: 50 - protein, 8 aa, Artificial Sequence
  A peptide containing D amino acids.
KXKXWXWX

SEQ ID NO: 51 - protein, 8 aa, Artificial Sequence
  A peptide containing D amino acids.
KXKXLXLX

SEQ ID NO: 52 - protein, 8 aa, Artificial Sequence
  A peptide containing D amino acids.
KXKXFXFX

SEQ ID NO: 53 - protein, 8 aa, Artificial Sequence
  A peptide containing D amino acids.
KXKXAXAX

SEQ ID NO: 54 - protein, 8 aa, Artificial Sequence
  A peptide containing D amino acids.
KXKXFXFX

SEQ ID NO: 55 - protein, 8 aa, Artificial Sequence
  A peptide containing D amino acids.
KXKXFXFX

SEQ ID NO: 56 - protein, 8 aa, Artificial Sequence
  A peptide containing D amino acids.
KXKXGXGX

SEQ ID NO: 57 - protein, 8 aa, Artificial Sequence
  A peptide containing D amino acids.
KXKXIXIX

SEQ ID NO: 58 - protein, 8 aa, Artificial Sequence
  A peptide containing D amino acids.
KXKXIXIX

SEQ ID NO: 59 - protein, 8 aa, Artificial Sequence
  A peptide containing D amino acids.
KXKXXXXX

SEQ ID NO: 60 - protein, 8 aa, Artificial Sequence
  A peptide containing D amino acids.
KXKXVXVX

SEQ ID NO: 61 - protein, 8 aa, Artificial Sequence
  A peptide containing D amino acids.
KXKXWXWX

SEQ ID NO: 62 - protein, 8 aa, Artificial Sequence
  A peptide containing D amino acids.
KXKXYXYX

SEQ ID NO: 63 - protein, 8 aa, Artificial Sequence
  A peptide containing D amino acids.
KXXXWXWX

SEQ ID NO: 64 - protein, 8 aa, Artificial Sequence
  A peptide containing D amino acids.
KXKXAXAX

SEQ ID NO: 65 - protein, 8 aa, Artificial Sequence
  A peptide containing D amino acids.
KXKXFXFX

SEQ ID NO: 66 - protein, 8 aa, Artificial Sequence
  A peptide containing D amino acids.
KXKXWXWX

SEQ ID NO: 67 - protein, 8 aa, Artificial Sequence
  A peptide containing D amino acids.
KXWXWXKX

SEQ ID NO: 68 - protein, 8 aa, Artificial Sequence
  A peptide containing D amino acids.
QXFXLXWX

SEQ ID NO: 69 - protein, 8 aa, Artificial Sequence
  A peptide containing D amino acids.
QXLXLXWX

SEQ ID NO: 70 - protein, 8 aa, Artificial Sequence
  A peptide containing D amino acids.
QXVXLXWX

SEQ ID NO: 71 - protein, 8 aa, Artificial Sequence
  A peptide containing D amino acids.
QXWXLXWX

SEQ ID NO: 72 - protein, 8 aa, Artificial Sequence
  A peptide containing D amino acids.
KXKXFXWX

SEQ ID NO: 73 - protein, 8 aa, Artificial Sequence
  A peptide containing D amino acids.
KXKXWXWX

SEQ ID NO: 74 - protein, 8 aa, Artificial Sequence
  A peptide containing D amino acids.
KXWXWXKX

SEQ ID NO: 75 - protein, 8 aa, Artificial Sequence
  A peptide containing D amino acids.
KXWXWXKX

SEQ ID NO: 76 - protein, 8 aa, Artificial Sequence
  A peptide containing D amino acids.
KXKXFXFX

SEQ ID NO: 77 - protein, 8 aa, Artificial Sequence
  A peptide containing D amino acids.
KXKXWXWX

SEQ ID NO: 78 - protein, 8 aa, Artificial Sequence
  A peptide containing D amino acids.
KXSXAXAX

SEQ ID NO: 79 - protein, 8 aa, Artificial Sequence
  A peptide containing D amino acids.
KXSXFXFX

SEQ ID NO: 80 - protein, 8 aa, Artificial Sequence
  A peptide containing D amino acids.
KXSXVXVX

SEQ ID NO: 81 - skipped

SEQ ID NO: 82 - protein, 8 aa, Artificial Sequence
  A peptide containing D amino acids.
KXSXWXWX

SEQ ID NO: 83 - protein, 8 aa, Artificial Sequence
  A peptide containing D amino acids.
KXSXYXYX

SEQ ID NO: 84 - protein, 8 aa, Artificial Sequence
  A peptide containing D amino acids.
KXWXWXRX

SEQ ID NO: 85 - protein, 8 aa, Artificial Sequence
  A peptide containing D amino acids.
KXKXKXLX

SEQ ID NO: 86 - protein, 8 aa, Artificial Sequence
  A peptide containing D amino acids.
KXQXKXWX

SEQ ID NO: 87 - protein, 8 aa, Artificial Sequence
  A peptide containing D amino acids.
KXQXKXLX

SEQ ID NO: 88 - protein, 8 aa, Artificial Sequence
  A peptide containing D amino acids.
KXSXKXLX

SEQ ID NO: 89 - protein, 8 aa, Artificial Sequence
  A peptide containing D amino acids.
KXQXKXWX

SEQ ID NO: 90 - protein, 8 aa, Artificial Sequence
  A peptide containing D amino acids.
KXQXKXWX

SEQ ID NO: 91 - protein, 8 aa, Artificial Sequence
  A peptide containing D amino acids.
KXKXKXLX

SEQ ID NO: 92 - protein, 8 aa, Artificial Sequence
  A peptide containing D amino acids.
KXQXKXLX

SEQ ID NO: 93 - protein, 8 aa, Artificial Sequence
  A peptide containing D amino acids.
KXSXKXLX

SEQ ID NO: 94 - protein, 8 aa, Artificial Sequence
  A peptide containing D amino acids.
KXQXKXWX

SEQ ID NO: 95 - protein, 8 aa, Artificial Sequence
  A peptide containing D amino acids.
KXQXKXWX

SEQ ID NO: 96 - protein, 8 aa, Artificial Sequence
  A peptide containing D amino acids.
KXQXKXWX

SEQ ID NO: 97 - protein, 8 aa, Artificial Sequence
  A peptide containing D amino acids.
KXQXKXWX

SEQ ID NO: 98 - protein, 8 aa, Artificial Sequence
  A peptide containing D amino acids.
KXQXKXWX

SEQ ID NO: 99 - protein, 8 aa, Artificial Sequence
  A peptide containing D amino acids.
KXWXWXHX

SEQ ID NO: 100 - protein, 8 aa, Artificial Sequence
  A peptide containing D amino acids.
KXWXWXKX

SEQ ID NO: 101 - protein, 8 aa, Artificial Sequence
  A peptide containing D amino acids.
KXKXLXLX

SEQ ID NO: 102 - protein, 8 aa, Artificial Sequence
  A peptide containing D amino acids.
KXQXKXWX

SEQ ID NO: 103 - protein, 8 aa, Artificial Sequence
  A peptide containing D amino acids.
KXKXHXHX

SEQ ID NO: 104 - protein, 8 aa, Artificial Sequence
  A peptide containing D amino acids.
KXKXQXQX

SEQ ID NO: 105 - protein, 8 aa, Artificial Sequence
  A peptide containing D amino acids.
KXKXRXRX

SEQ ID NO: 106 - protein, 8 aa, Artificial Sequence
  A peptide containing D amino acids.
KXKXRXRX

SEQ ID NO: 107 - protein, 8 aa, Artificial Sequence
  A peptide containing D amino acids.
KXKXSXLX

SEQ ID NO: 108 - protein, 8 aa, Artificial Sequence
  A peptide containing D amino acids.
KXQXKXWX

SEQ ID NO: 109 - protein, 8 aa, Artificial Sequence
  A peptide containing D amino acids.
XRXWXEXW

SEQ ID NO: 110 - protein, 8 aa, Artificial Sequence
  A peptide containing D amino acids.
XRXWXHXW

SEQ ID NO: 111 - protein, 8 aa, Artificial Sequence
  A peptide containing D amino acids.
XRXWXKXW

SEQ ID NO: 112 - protein, 8 aa, Artificial Sequence
  A peptide containing D amino acids.
XRXWXSXW

SEQ ID NO: 113 - protein, 6 aa, Artificial Sequence
  A peptide containing D amino acids.
KXVXAX

SEQ ID NO: 114 - protein, 6 aa, Artificial Sequence
  A peptide containing D amino acids.
KXIXAX

SEQ ID NO: 115 - protein, 6 aa, Artificial Sequence
  A peptide containing D amino acids.
RXWXWX

SEQ ID NO: 116 - protein, 6 aa, Artificial Sequence
  A peptide containing D amino acids.
KXQXLX

SEQ ID NO: 117 - protein, 6 aa, Artificial Sequence
  A peptide containing D amino acids.
KXKXLX

SEQ ID NO: 118 - protein, 6 aa, Artificial Sequence
  A peptide containing D amino acids.
KXQXLX

SEQ ID NO: 119 - protein, 6 aa, Artificial Sequence
  A peptide containing D amino acids.
KXKXLX

SEQ ID NO: 120 - protein, 6 aa, Artificial Sequence
  A peptide containing D amino acids.
KXQXLX

SEQ ID NO: 121 - protein, 6 aa, Artificial Sequence
  A peptide containing D amino acids.
KXKXLX

SEQ ID NO: 122 - protein, 6 aa, Artificial Sequence
  A peptide containing D amino acids.
KXQXLX

SEQ ID NO: 123 - protein, 6 aa, Artificial Sequence
  A peptide containing D amino acids.
KXQXLX

SEQ ID NO: 124 - protein, 6 aa, Artificial Sequence
  A peptide containing D amino acids.
KXQXLX

SEQ ID NO: 125 - protein, 6 aa, Artificial Sequence
  A peptide containing D amino acids.
KXKXLX

SEQ ID NO: 126 - protein, 6 aa, Artificial Sequence
  A peptide containing D amino acids.
KXKXLX

SEQ ID NO: 127 - protein, 6 aa, Artificial Sequence
  A peptide containing D amino acids.
KXYXWX

SEQ ID NO: 128 - protein, 6 aa, Artificial Sequence
  A peptide containing D amino acids.
KXKXLX

SEQ ID NO: 129 - protein, 6 aa, Artificial Sequence
  A peptide containing D amino acids.
KXQXLX

SEQ ID NO: 130 - protein, 6 aa, Artificial Sequence
  A peptide containing D amino acids.
KXQXRX

SEQ ID NO: 131 - protein, 6 aa, Artificial Sequence
  A peptide containing D amino acids.
KXQXRX

SEQ ID NO: 132 - protein, 6 aa, Artificial Sequence
  A peptide containing D amino acids.
KXIXIX

SEQ ID NO: 133 - protein, 8 aa, Artificial Sequence
  A peptide containing D amino acids.
RXRXLXLX

SEQ ID NO: 134 - protein, 8 aa, Artificial Sequence
  A peptide containing D amino acids.
XKXWXWXW

SEQ ID NO: 135 - protein, 8 aa, Artificial Sequence
  A peptide containing D amino acids.
XRXWXWXW

SEQ ID NO: 136 - protein, 8 aa, Artificial Sequence
  A peptide containing D amino acids.
XRXWXWXW

SEQ ID NO: 137 - protein, 8 aa, Artificial Sequence
  A peptide containing D amino acids.
XWXRXWXW

SEQ ID NO: 138 - protein, 8 aa, Artificial Sequence
  A peptide containing D amino acids.
XWXRXWXW

SEQ ID NO: 139 - protein, 8 aa, Artificial Sequence
  A peptide containing D amino acids.
RXKXLXLX

SEQ ID NO: 140 - protein, 8 aa, Artificial Sequence
  A peptide containing D amino acids.
KXRXLXLX

SEQ ID NO: 141 - protein, 6 aa, Artificial Sequence
  A peptide containing D amino acids.
XKXWXW

SEQ ID NO: 142 - protein, 8 aa, Artificial Sequence
  A peptide containing D amino acids.
QXWXLXWX

SEQ ID NO: 143 - protein, 8 aa, Artificial Sequence
  A peptide containing D amino acids.
XEXWXWXW

SEQ ID NO: 144 - protein, 8 aa, Artificial Sequence
  A peptide containing D amino acids.
WXKXWXWX

(19) United States
(12) Patent Application Publication
(10) Pub. No.: US 2005/0107289 A1
(43) Pub. Date: May 19, 2005

(54) Anti-microbial peptides and compositions

(75) Inventors: M. Reza Ghadiri, San Diego, CA (US); Hui-Sun Kim, El Cerrito, CA (US); Sara Fernandez-Lopez, Menlo Park, CA (US); Keith Wilcoxen, Cambridge, MA (US)

Correspondence Address: Lisa A. Haile, J.D., Ph.D.;GRAY CARY WARE & FREIDENRICH LLP, Suite 1100, 4365 Executive Drive, San Diego, CA, 92121-2133, US

(73) Assignee: THE SCRIPPS RESEARCH INSTITUTE

(21) Appl. No.: 10/886,165

(22) Filed: Jul. 6, 2004

Related U.S. Application Data
(63) Continuation of application No. 10/141,688, filed on May 6, 2002, now abandoned.
(60) Provisional application No. 60/288,990, filed on May 4, 2001.

(51) Int. Cl. A61K 38/12

(52) U.S. Cl. 514/9

(57) ABSTRACT

The present invention provides novel anti-microbial agents and compositions that include cyclic peptides having an amino acid sequence of alternating D- and L-α-amino acids. Alternatively, the cyclic peptides are made from β-amino acids.

This application claims priority from provisional application U.S. Ser. No. 60/288,990, filed May 4, 2001.

Work relating to this application was supported by a grant from the National Institutes of Health (GM 52190). The government may have certain rights in the invention.

### FIELD OF THE INVENTION

The present invention provides cyclic peptide anti-microbials. Anti-microbial peptides and compositions of the invention can quickly kill microbes causing an infection without causing substantial or undesired harm to mammalian cells. The cyclic peptides of the invention may be composed of a sequence of α-amino acids that have a repeating D, L-backbone chirality, or homochiral β-amino acids.

### BACKGROUND OF THE INVENTION

Antibiotic resistance is gaining in importance as a medical problem because more microorganisms are becoming resistant to greater numbers of antibiotics. See, Antibiotic Resistance, A Growing Threat, website at fda.gov/oc/opacom/hottopics/anti_resist.html; P. J. Koplan et al., Preventing emerging infections diseases. A Strategy for the 21^{st} Century, U.S. Department of Health & Human Services, Center for Disease Control & Prevention, Atlantic, Ga. (1998). For example, some strains of Staphylococcus aureus have developed resistance to many distinct antibiotics and are now described as having a “multi-resistant” phenotype. Unfortunately, due to extensive use of antibiotics, such multi-resistant strains of microorganisms are now found in the general population and within hospitals. Hospital-acquired bacterial infections occur in 5% of patients admitted to the hospital (about 2 million patients per year in the United States). Such infections cause an estimated 20,000 deaths per year, and contribute to an additional 60,000 hospital deaths every year. It is estimated that such infections cause about 7.5 million added days of hospitalization and cost an extra $1 billion dollars in health care costs each year.

“New” antibiotics are often structurally related to, or structurally derived from, a previous generation of antibiotics. For example, cephalosporin is structurally related to penicillin. While these structural analogs of known antibiotics can be successful for a time, increasing reports of resistance often develop as the “new” antibiotic becomes widely used. The progression of β-lactam antibiotics illustrates this problem. Since the introduction of penicillin in the 1940's, microbes have evolved resistance to β-lactam antibiotics, typically by acquired or enhanced catabolism of these drugs through the enzyme β-lactamase that is found in many bacterial strains. While researchers have made successive changes to the basic β-lactam antibiotic structure in order to generate new antibiotics that are more resistant to degradation by β-lactamases, microorganisms have responded by producing different types or larger amounts of β-lactamases.

Approximately seventy percent of hospital-acquired bacterial infections are resistant to at least one of the most commonly prescribed antibiotics. Such resistance often develops because commonly used antibiotics act comparatively slowly on the metabolism or cellular structure of the bacteria. For example, a common mechanism by which β-lactam antibiotic-resistance develops is via plasmid amplification of an encoded protein that can breakdown the β-lactam antibiotic. When β-lactam antibiotics are routinely prescribed, as often occurs in hospitals, a small segment of the bacterial population that is resistant to such antibiotics can propagate, the plasmids encoding the resistance function can amplify, and the highly resistant strain of bacteria can spread throughout the hospital.

This problem has been addressed by the production of new generations of antibiotics. For example, valinomycin is a cyclic depsipeptide with an alternating D-D-L-L chiral motif that employs ester linkages within the ring structure. However, the activity, selectivity, in vivo stability, toxicity and bioavailability of valinomycin and other such peptides are not optimal. Accordingly, there is a need for fast-acting, non-toxic anti-microbial agents to combat microbial infections, especially infections that are resistant to currently available antibiotics, which are easily and cheaply manufactured.

### SUMMARY OF THE INVENTION

The present invention provides new, fast-acting anti-microbial cyclic peptides and compositions for treating and/or preventing microbial infections. The present anti-microbial agents are highly effective for many microbial species, including multiply-resistant bacteria as well as lethal methicillin-resistant and vancomycin-resistant microbial strains. Cyclic peptides are fast-acting, proteolytically stable and easy to synthesize. In many embodiments, substantially no microbes can be detected within just a few hours of exposure to the cyclic peptides of the invention. Still others do not cause substantial undesired lysis of mammalian cells, for example, as measured by hemolysis of erythrocytes.

The invention provides anti-microbial cyclic peptides and pharmaceutical compositions thereof for human and veterinary applications wherein the cyclic peptides comprise a sequence of from four to about sixteen alternating D- and L-α-amino acids, and wherein the cyclic peptide has preferential cytotoxic activity against the target microbial organism and does not have substantial undesired activity against mammalian cells. Activity against mammalian cells can be measured, for example, by the ability of peptides to cause hemolysis of mammalian red blood cells. Such cyclic peptides and pharmaceutical compositions can be used for treating or preventing a microbial infection in a mammal. Unlike many anti-microbial agents, preferred cyclic peptides of the invention have been shown to be bactericidal, and not merely bacteristatic at minimum inhibitory concentrations.

The cyclic peptides of the invention generally have a minimum inhibitory concentration at which substantially no cells of at least one target microbe grow in vitro that is less than one half the peptide concentration needed to cause 50% hemolysis of mammalian red blood cells. In other embodiments, the minimum inhibitory concentration can be less than one quarter to less than one fifth the peptide concentration needed to cause 50% hemolysis of mammalian red blood cells. In other embodiments, the minimum inhibitory concentration is less than at least one tenth to less than at least one twentieth the peptide concentration needed to cause 50% hemolysis of mammalian red blood cells.

A large variety of microbial infections can be treated with the cyclic peptides of the invention. For example, the target microbial organism can be a bacterial strain, a yeast strain, a fungal strain, a single-cell organism, a single-cell parasite or a related organism. In one embodiment, the target microbial organisms are gram-positive bacteria or gram-negative bacteria.

The cyclic peptides of the invention are believed to self-assemble into supramolecular structures within or by association with microbial membranes. Such supramolecular structures can be, for example, nanotubes, barrels or groups of associated, axially parallel nanotubes, carpets of associated nanotubes, or mixtures thereof. These types of supramolecular structures can induce microbial cell membrane permeation, depolarization or disruption (e.g., lysis) selectively over mammalian cell membrane permeation, depolarization or disruption.

Cyclic peptides of the invention may have a plurality of amino acids having side chains with affinity for biomolecules integral to microbial cell membranes. Such biomolecules can facilitate selective assembly of the cyclic peptides into supramolecular structures on or within microbial membranes. Most mammalian cell membranes generally do not have the same biomolecular composition as most microbial membranes. Cyclic peptides of the invention will not preferably associate with mammalian cell membrane over microbial cell membranes at desired anti-microbial concentrations.

The cyclic peptides can have a half-life in the bloodstream of the mammal of about six hours or less, at an amount that is effective against microbial infections.

Such an effective amount is an amount of the cyclic peptide that is sufficient to induce death or lysis of microbial cells in single or divided doses without inducing an undesired amount of death or lysis of mammalian cells. Preferred cyclic peptides of the invention induce substantially no hemolysis of red blood cells at anti-microbially effective doses.

In some embodiments, the cyclic peptides of the invention can be administered in a single dosage and yet are effective for treating microbial infections. Alternatively, the cyclic peptides of the invention can be administered continuously or in multiple doses over a period of one to ten days. Effective amounts of the present cyclic peptides include about 0.1 mg/kg to about 100 mg/kg of body weight, alternatively about 0.5 mg/kg to about 50 mg/kg of body weight, about 1.0 mg/kg to about 30 mg/kg of body weight, and other amounts set forth herein.

The cyclic peptides of the invention generally have about 25% to about 88% D- and/or L-polar amino acids. In some embodiments, the percentage of polar amino acids can be from about 33% or 50% to about 65% or 88% of the total number of D- and L-amino acids. Thus, for example, an eight residue cyclic peptide of the invention can have at least one, alternatively, two to seven polar D- and L-amino acids. Other eight residue cyclic peptides will have three to five polar D- and L-amino acids for example. In some embodiments, for example, six residue cyclic peptides of the invention can have two to five polar D- and L-amino acids. Other six residue cyclic peptides may have three to four polar D- and L-amino acids. At least one of these polar D- or L-amino acids may be adjacent to at least one other polar D- or L-amino acid. Alternatively, at least one polar D- or L-amino acid may be adjacent only to nonpolar D- or L-amino acids.

A variety of polar amino acids are available to one of skill in the art. Examples of polar D- and L-α-amino acids that can be utilized in the peptides of the invention include the D- or L-enantiomers of serine, threonine, asparagine, glutamine, aspartic acid, cysteine, homocysteine, glutamic acid, histidine, arginine, lysine, hydroxylysine or ornithine.

The cyclic peptides of the invention generally have about 25% to about 88% D- and/or L-ionizable amino acids. In some embodiments, the percentage of ionizable amino acids can be from about 33% or 50% to about 65% or 88% of the total number of D- and L-amino acids. Thus, for example, a six or eight residue cyclic peptide can have at least one, or alternatively two or three or more ionizable D- and/or L-amino acids. In other embodiments, the cyclic peptides of the invention can have four to six ionizable D- and/or L-amino acids. Such an ionizable D- or L-amino acid can be adjacent to at least one other polar or ionizable D- or L-amino acid. Alternatively, the cyclic peptides of the invention can have at least one ionizable D- or L-amino acid that is adjacent only to nonpolar D- or L-amino acids.

Many types of ionizable amino acids are available to one of skill in the art and the invention contemplates all such ionizable amino acids. Examples of ionizable D- and/or L-amino acids include the D- or L-enantiomers of arginine, aspartic acid, glutamic acid, histidine, lysine, hydroxylysine or ornithine.

The cyclic peptides of the invention can have nonpolar D- and/or L-amino acid residues. The cyclic peptides of the invention generally have about 12% to about 75% D- and/or L-nonpolar amino acids. In some embodiments, the percentage of nonpolar amino acids can be from about 50% to about 67% or 75% of the total number of D- and/or L-amino acids. Thus, for example, an eight residue cyclic peptide of the invention can have at least one, alternatively, two to seven nonpolar D- and/or L-amino acids. Other eight residue cyclic peptides may have three to five nonpolar D- and/or L-amino acids. In some embodiments, for example, six residue cyclic peptides of the invention have two to five nonpolar D- and/or L-amino acids. Other six residue cyclic peptides may have three to four nonpolar D- and/or L-amino acids. At least one of these nonpolar D- and/or L-amino acids may be adjacent to at least one other nonpolar D- and/or L-amino acid. Alternatively, at least one nonpolar D- or L-amino acid may be adjacent only to polar D- or L-amino acids.

Many types of nonpolar amino acids are available to one of skill in the art and the invention contemplates all such nonpolar amino acids. Examples of nonpolar amino acids include the D- and L-enantiomers of alanine, valine, isoleucine, leucine, methionine, norleucine, phenylalanine, tyrosine or tryptophan.

In addition to the amino acids found naturally in proteins, other naturally occurring amino acids may be used, as well as non-naturally occurring and synthetic amino acids.

In one embodiment, the cyclic peptides of the invention can have an amino acid sequence having formula I: 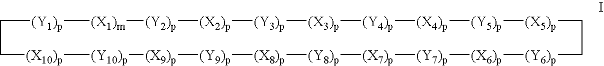
wherein:
   m is an integer ranging from 1 to 7;
   each p is separately an integer ranging from 0 to 7;
   each X₁, X₂, X₃, X₄, X₅, X₆, X₇, X₈, X₉, and X₁₀ is separately a polar D- or L-α-amino acid; and
   each Y₁, Y₂, Y₃, Y₄, Y₅, Y₆, Y₇, Y₈, Y₉, and Y₁₀ is separately nonpolar D- or L-α-amino acid; and
wherein the cyclic peptide has an even number of from four to about sixteen alternating D- and L-α amino acids.

In another embodiment, the cyclic peptides of the invention can have an amino acid sequence having formula II: 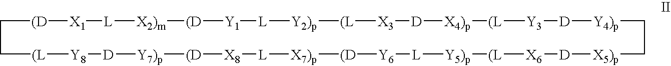
wherein:
   m is an integer ranging from 1 to 7;
   each p is separately an integer ranging from 0 to 7;
   each X₁, X₂, X₃, X₄, X₅, X₆, X₇, and X₈ is separately a polar D- or L-α-amino acid;
   each Y₁, Y₂, Y₃, Y₄, Y₅, Y₆, Y₇, and Y₈ is separately nonpolar D- or L-α-amino acid; and
wherein the cyclic peptide has an even number of from four to about sixteen alternating D- and L-α amino acids.

In yet another embodiment, the cyclic peptides of the invention can have an amino acid sequence having formula III: 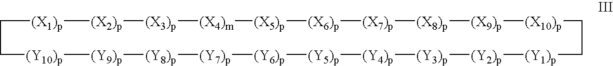
wherein:
   m is an integer ranging from 1 to 7;
   each p is separately an integer ranging from 0 to 7;
   each X₁, X₂, X₃, X₄, X₅, X₆, X₇, X₈, X₉, and X₁₀ is separately a polar D- or L-α-amino acid;
   each Y₁, Y₂, Y₃, Y₄, Y₅, Y₆, Y₇, Y₈, Y₉, and Y₁₀ is separately nonpolar D- or L-α-amino acid; and
wherein the cyclic peptide has an even number of from four to about sixteen alternating D- and L-α amino acids.

In yet another embodiment, the cyclic peptide has an amino acid sequence of formula IVa or IVb: 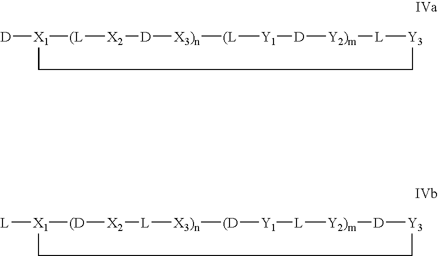
wherein:
   n is an integer ranging from 0 to 4;
   m is an integer ranging from 1 to 7;
   X₁, X₂ and X₃ are each separately a polar amino acid;
   Y₁, Y₂ and Y₃ are each separately nonpolar amino acid; and
wherein the cyclic peptide has an even number of from four to about sixteen alternating D- and L-α amino acids.

In another embodiment, the cyclic peptide has an amino acid sequence of formula Va or Va: 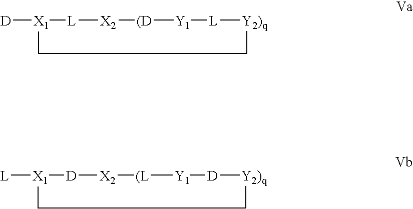
wherein:
   q is an integer ranging from 2 to 7;
   X₁ and X₂ are separately polar amino acids;
   Y₁ and Y₂ are separately nonpolar amino acids.

The claimed cyclic peptides of the above formulae, however, exclude those composed entirely of nonpolar amino acids. Moreover, a peptide having any one of the following sequences may be excluded from one or more of the above formulae:

cyclo-[-(Gln-D-Ala)₄-]
cyclo-[-(Gln-D-Leu)₄-]
cyclo-[-(Gln-D-Val)₄-]
cyclo-[-(Phe-D-Leu)₄-]
cyclo-[-(Phe-D-Ala)₄-]
cyclo-[-(Gln-D-Ala-Glu-D-Ala)₂-]
cyclo-[-(Gln-D-Phe-Glu-D-Phe)₂-]
cyclo-[-(Gln-D-Ala-Glu-D-Ala)₃-]
cyclo-[-(Trp-D-Leu)₃-Gln-D-Leu-]
cyclo-[-Gln-(D-Leu-Trp)₄-D-Leu-]
cyclo-[-Lys-D-Leu-Lys-D-Leu-Lys-D-Leu-Lys-D-Leu];
cyclo-[-Arg-D-Leu-Arg-D-Leu-Arg-D-Leu-Arg-D-Leu-];
cyclo-[-Glu-D-Leu-Glu-D-Leu-Glu-D-Leu-Glu-D-Leu-].

Additionally, the following sequences may or may not be excluded from the above formulae and associated compositions:

cyclo-[-D-Arg-Gln-D-Arg-Ala-D-Trp-Leu-D-Trp-Trp-];
cyclo-[-D-Arg-Gln-D-Arg-Leu-D-Trp-Leu-D-Trp-Trp-];
cyclo-[-D-Arg-Gln-D-Arg-Val-D-Trp-Leu-D-Trp-Trp-];
cyclo-[-D-Arg-Gln-D-Arg-Phe-D-Trp-Leu-D-Trp-Trp-];
or
cyclo-[-D-Arg-Gln-D-Arg-Trp-D-Trp-Leu-D-Trp-Trp-].

Cyclic peptides having any of the above sequences, however, including any of the excluded sequences, may be included within or excluded from pharmaceutical compositions and peptides of the present invention.

Cyclic peptides often have, for example, about four to about sixteen D- and L-α-amino acids. In other embodiments, the cyclic peptides have about six to about ten or twelve D- and L-α-amino acids. In still other embodiments, cyclic peptides of about six or eight D- and L-α-amino acids are employed.

The pharmaceutical compositions of the invention can include an effective amount of at least one of the cyclic peptides of the invention, or two or more different cyclic peptides of the invention. These compositions also include a pharmaceutically effective carrier.

According to the invention, the cyclic peptides need not be made from D- or L-α-amino acids and can alternatively have a sequence of from three to about ten homochiral β-amino acids. Such β-amino acids are available to one of skill in the art. Beta amino acids can be substituted at the α- or β-carbons by one to two substituents. Mono-substituted beta amino acids of either S or R chirality can be employed for the construction of cyclic β-peptides, provided that the cyclic β-peptide is homochiral. Disubstituted β-amino acids employed in the homochiral β-peptides of the present invention have the relative R,R or S,S diastereomeric configuration, provided that the cyclic β-peptide is homochiral. In other words, the β-amino acids of the cyclic β-peptides of the present invention should be homochiral, i.e., the substituents at the α and/or β backbone carbons should be all S and/or S,S, or all R and/or R,R. Cyclic peptides having β-amino acids generally have at least one β-amino acid with at least one polar side chain. Preferred β-peptides cause substantially no undesirable lysis of mammalian cells.

In one embodiment, the cyclic β-peptides of the invention can have an amino acid sequence of formula VI: 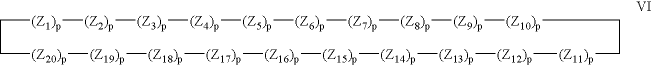
wherein:
   each p is separately an integer ranging from 0 to 7;
   each Z₁, Z₃, Z₅, Z₇, Z₉, Z₁₁, Z₁₃, Z₁₅, Z₁₇, and Z₁₉ is separately a monosubstituted β-amino acid;
   each Z₂, Z₄, Z₆, Z₈, Z₁₀, Z₁₂, Z₁₄, Z₁₆, Z₁₈, and Z₂₀ is separately a disubstituted β-amino acid; and
wherein the cyclic β-peptide has a sequence of from three to about ten homochiral β-amino acids. The invention therefore also contemplates pharmaceutical compositions that include an effective amount of at least one cyclic peptide, or two or more different cyclic peptides, wherein these cyclic peptide(s) have a sequence of from three to about ten homochiral β-amino acids, for example, as provided by formula VI.

The invention also provides a method of treating a microbial infection in humans and other animals, which comprises contacting microbial cells with a cyclic peptide comprising a sequence of from four to about sixteen amino acids, wherein the sequence has alternating D- and L-α-amino acids, in an amount sufficient to induce microbial cell death without inducing an undesirable amount of mammalian or animal cell death. Such cyclic peptides can alternatively have a sequence of from three to about ten homochiral β-amino acids.

The invention further provides a method of identifying cyclic peptides selectively cytotoxic to a target cell-type comprising screening one or more cyclic peptides for induction of cell death in target cells without induction of substantial or undesired cell death in a second cell type. Such cyclic peptides may comprise, for example, an alternating D- and L-α amino acid sequence of between four and about sixteen amino acids. Alternatively, such cyclic peptides may have a sequence of from three to about ten homochiral β-amino acids.

The invention also provides a method of identifying cyclic peptides selectively cytotoxic to a target cell-type comprising: (a) making a combinatorial library of cyclic peptides, wherein each cyclic peptide in the combinatorial library comprises an alternating D- and L-α amino acid sequence of between four and about sixteen amino acids; and (b) screening cyclic peptides from the combinatorial library for induction of cell death in target cells without induction of substantial or undesired cell death in a second cell type. Alternatively, such cyclic peptides may have a sequence of from three to about ten homochiral β-amino acids. The library can be used to generate single cyclic peptides or mixtures of cyclic peptides. Mixtures of cyclic peptides that show anti-microbial activity can then be further screened to identify one or more anti-microbially active cyclic peptides in one or more of the mixtures, which can then be isolated or synthesized and re-tested for induction of cell death in target cells without induction of substantial or undesired cell death in a second cell type.

The invention further provides a method of identifying cyclic peptides selectively cytotoxic to a target cell-type. This method involves the step of rationally designing at least one cyclic peptide comprising an alternating D- and L-α amino acid sequence of between four and sixteen amino acids. Alternatively, such cyclic peptides may have a sequence of from three to about ten homochiral β-amino acids. The method further involves screening such rationally designed cyclic peptides for induction of cell death in target cells without induction of substantial or undesired cell death in a second cell type.

Rationally designing a cyclic peptide can involve identifying at least one effective cyclic peptide from a combinatorial library that can induce cell death in target cells without inducing substantial or undesired cell death in a second cell type and exchanging at least one amino acid for a different amino acid in the alternating D- and L-α amino acid sequence of the effective cyclic peptide to generate a rationally designed cyclic peptide. The rationally designed cyclic peptide can then be screened for induction of cell death in target cells without inducing substantial or undesired cell death in a second cell type. Alternatively, such cyclic peptides may have a sequence of from three to about ten homochiral β-amino acids.

In these types of screening methods, the target cell-type can be a microbe and the second cell type can be a mammalian cell, for example, a mammalian red blood cell. Cell death of the second cell type can be detected by detecting hemolysis of red blood cells.

The above methods can further include screening a third cell type. Such screening can include determining whether a peptide induces substantial or undesired cell death in the third cell type. The method can also include determining the minimum inhibitory dose at which a peptide can kill substantially all, or inhibit the growth, of the target cells.

Screening can be performed in vitro by separately contacting a peptide with the target cell type and with other cell types (e.g., a second or third or other cell type). Alternatively, screening can be performed in vivo by administering at least one peptide to a test animal comprising the target cell type and another cell type or types and determining whether the peptide is toxic to the target cell type but does not have substantial or undesired toxicity to another cell type or types. Such a method can also include determining whether the peptide adversely affects the health of the animal. Determining whether the peptide adversely affects the health of the animal can include examining the bodily fluids or the anatomy of the animal by pathological or histological methods.

The invention further provides a method of identifying cyclic peptides capable of selective association with one or more target biomolecules on a selected cell surface, comprising contacting a solution of cyclic peptides, each peptide comprising between four to about sixteen amino acids in an alternating D- and L-α amino acid sequence, or a peptide comprising from three to about ten β-amino acids, with the target biomolecule(s) and determining, for example, whether the peptides spontaneously assemble into a supramolecular structure that selectively associates with the biomolecule(s). The target biomolecule can be displayed, for example, on the surface of a living cell or on the surface of a liposome. Alternatively, the peptide can be contacted with the target biomolecule(s). The method can further include determining the structure of the peptides that spontaneously assemble into the supramolecular structure that selectively associates with the biomolecule(s).

The present invention also provides methods of evaluating or confirming therapeutically effective dosages for treating a microbial infection with a cyclic peptide having an amino acid sequence of alternating D- and L-α-amino acids, or a cyclic peptide comprising from three to about ten β-amino acids, that includes determining the minimum inhibitory concentration of the cyclic peptide at which substantially no bacteria grow in vitro.

The present invention also provides a composition comprising one or more of the present cyclic peptides with one or more other anti-microbial agents. Such agents include penicillin, vancomycin, erythromycin and other therapeutic agents.

Mammals and birds may be treated by the methods and compositions described and claimed herein. Such mammals and birds include humans, dogs, cats, and livestock, for example, horses, cattle, sheep, goats, chickens, turkeys and the like.

### BRIEF DESCRIPTION OF THE FIGURES

FIG. 1 provides a schematic drawing of the self-assembly of the present cyclic peptides into nanotubules. An eight-residue cyclic D, L-α-peptide with alternating D- and L-amino acids is depicted to the left, with emphasis on its flat ring-shaped conformation. Side chains (R) decorate the outside surface of the cyclic peptide. Upon self-assembly, a series of cyclic peptides align and are believed to undergo intermolecular hydrogen bonding to form a tubular structure referred to herein as a nanotube (center). Self-assembly is believed to be directed by inter-subunit backbone-backbone hydrogen-bonding resulting in a β-sheet-like open-ended hollow tubular supramolecular structure. This β-sheet like hydrogen bonding pattern is shown to the right. For clarity most side chains are omitted.

FIG. 2 illustrates the modes of membrane permeation that are accessible to peptide supramolecular structures. Depending on the composition and sequence of amino acids employed in the cyclic peptides, supramolecular structures that may be formed can interact with the membranes of cells through, for example, supramolecular assembly of (a) pores, (b) barrel stave structures, or (c) carpet-like modes of action. Cyclic peptides are depicted as ring structures.

FIG. 3 graphically illustrates the rate of membrane depolarization of intact S. aureus (ATCC 25923) upon exposure to cyclic peptide SEQ ID NO:8 (open circles), cyclic peptide SEQ ID NO:18 (open triangles), and cyclic peptide SEQ ID NO:26 (open squares). Membrane depolarization was monitored by the change in the fluorescence emission intensity of the membrane potential-sensitive dye diSC₃. The excitation wavelength (λₑₓ) was 622 nm and the emission wavelength (λₑₘ) was 670 nm. Peptides were added at t=1 min and aliquots taken at t=4 min to obtain viable bacteria counts. In all cases at least a 1000 fold reduction in viability upon the addition of the peptides was observed.

FIG. 4 provides a plot of the apparent proton transport (FIG. 4a) and the carboxyfluoroscein release (FIG. 4b) mediated by peptide SEQ ID NO:11 (cyclo-[Gln-D-Lys-(Trp-D-Leu)₂-Trp-D-Lys-]), as expressed in fractional fluorescence changes as a function time. For FIG. 4b, the peptide was added at about 100 seconds, and the detergent triton X-100 was added at about 200 seconds.

FIG. 5 provides an attenuated total reflectance (ATR) infrared (IR) spectra for analyzing the orientation of peptide SEQ ID NO:11 (cyclo-[-Gln-D-Lys-(Trp-D-Leu)₂-Trp-D-Lys-] in DMPC multibilayers. The solid trace indicates absorbance of parallel-polarized light; the dashed trace provides absorbance of perpendicular-polarized light.

FIG. 6 provides a dose-effect curve illustrating the in vivo efficacy of three cyclic peptides as anti-microbial compounds. As illustrated, the survival rate of mice challenged with lethal doses of methicillin-resistant Staphylococcus aureus is dramatically improved as the dosage of the cyclic peptide is increased. Three different cyclic peptides were tested: (a) peptide SEQ ID NO:18 (PD₅₀=5±2 mg/kg), (b) peptide SEQ ID NO:17 (PD₅₀=6±2 mg/kg), and (c) peptide SEQ ID NO:26 (PD₅₀=10±2 mg/kg). PD50 is the protective dose where 50% of animals survive.

FIG. 7 provides a structural comparison of supramolecular structures composed of: (a) cyclic β-tetrapeptides, and (b) cyclic D, L-α-octapeptides. This figure illustrates that, due to the unidirectional arrangement of the polar backbone amide groups, cyclic β-tetrapeptide supramolecular structures may possess a macrodipole moment reminiscent of an α-helix, while cyclic D, L-α-octapeptide supranolecular structures will, under most circumstances, not have such a net dipole moment. For clarity most side chains are omitted from the nanotube structures depicted in FIGS. 7a and 7b.

FIG. 8a provides a “time-killing curve” illustrating the number of surviving S. aureus (MRSA, ATCC 33591) in log cfu/ml as a function of time after exposure to one of the following peptides at MIC concentrations: cyclo[KHLWLW] (closed circles), cyclo[KRKWLWLW] (closed triangles) or cyclo[KQRWLWLW] (open squares), where the underlining indicates that the amino acid is a D-amino acid.

FIG. 8b provides a “time-killing curve” illustrating the number of surviving E. coli JM109 (DE3) in log cfu/ml as a function of time after exposure to one of the following peptides at the MIC concentration: cyclo[KRKWLWLW] (closed triangles), cyclo[KQRWLWLW] (closed circles), cyclo[KHLWLW] (closed squares) or cyclo[KKLWLW] (open squares), where the underlining indicates that the amino acid is a D-amino acid.

FIG. 9 provides a “time-killing curve” illustrating the number of surviving S. aureus (MRSA) (ATCC 33592)(log cfu/ml) as a function of time after exposure to the cyclo[KSKWLWLW] peptide at 37° C., where the underlining indicates that the amino acid is a D-amino acid. The effects of several concentrations of the cyclo[KSKWLWLW] peptide on separate cultures are shown.

FIG. 10 provides a “time-killing curve” illustrating the number of surviving S. aureus (MRSA) (ATCC 33592)(log cfu/ml) as a function of time after exposure of the cyclo[KSKWLWLW] peptide at room temperature, where the underlining indicates that the amino acid is a D-amino acid. The effects of several concentrations of the cyclo[KSKWLWLW] peptide on separate cultures are shown.

FIG. 11 provides a “time-killing curve” illustrating the number of surviving Eneterococus faecium SP180 (cfu/ml) as a function of time after exposure to the cyclo[KSKWLWLW] peptide at 37° C., where the underlining indicates that the amino acid is a D-amino acid. E. faecium SP180 is vancomycin resistant (vanA) and multiply resistant to several FDA approved antibiotics. The effects of several concentrations of the cyclo[KSKWLWLW] peptide on separate cultures are shown.

FIG. 12 provides a “time-killing curve” illustrating the number of surviving Eneterococus faecium SP 180 (cfu/ml) as a function of time after exposure to the cyclo[KSKWLWLW] peptide at room temperature, where the underlining indicates that the amino acid is a D-amino acid. E. faecium SP 180 is vancomycin resistant (vanA) and multiply resistant to several FDA approved antibiotics. The effects of several concentrations of the cyclo[KSKWLWLW] peptide on separate cultures are shown.

FIG. 13 provides a “time-killing curve” illustrating the number of surviving S. aureus (MRSA) (ATCC 33592)(cfu/ml) as a function of time after exposure to the cyclo[RRKWLWLW] peptide at 37° C., where the underlining indicates that the amino acid is a D-amino acid. The effects of several concentrations of the cyclo[RRKWLWLW] peptide on separate cultures are shown.

FIG. 14 provides a “time killing curve” illustrating the number of surviving S. aureus (MRSA) (ATCC 33592)(cfu/ml) as a function of time after exposure to the cyclo[RRKWLWLW] peptide at room temperature, where the underlining indicates that the amino acid is a D-amino acid. The effects of several concentrations of the cyclo[RRKWLWLW] peptide on separate cultures are shown.

FIG. 15 provides a “time-killing curve” illustrating the number of surviving Eneterococus faecium SP180 (cfu/ml) as a function of time after exposure to the cyclo[RRKWLWLW] peptide at 37° C., where the underlining indicates that the amino acid is a D-amino acid. E. faecium SP180 is vancomycin resistant (vanA) and multiply resistant to several FDA approved antibiotics. The effects of several concentrations of the cyclo[RRKWLWLW] peptide on separate cultures are shown.

FIG. 16 provides a “time-killing curve” illustrating the number of surviving Eneterococus faecium SP180 (cfu/ml) as a function of time after exposure to the cyclo[RRKWLWLW] peptide at room temperature, where the underlining indicates that the amino acid is a D-amino acid. E. faecium SP180 is vancomycin resistant (vanA) and multiply resistant to several FDA approved antibiotics. The effects of several concentrations of the cyclo[RRKWLWLW] peptide on separate cultures are shown.

FIG. 17 provides a “time-killing curve” illustrating the number of surviving S. aureus (MRSA) (ATCC 33592)(cfu/ml) as a function of time after cells were exposed to penicillin G at 37° C. The effects of several concentrations penicillin G on separate cultures are shown.

FIG. 18 provides a “time-killing curve” illustrating the number of surviving S. aureus (MRSA) (ATCC 33592)(cfu/ml) as a function of time after cells were exposed to penicillin G at room temperature. The effects of several concentrations of penicillin G on separate cultures are shown.

FIG. 19 provides a “time-killing curve” illustrating the number of surviving Eneterococus faecium SP 180 (cfu/ml) as a function of time after cells were exposed to penicillin G at 37° C. E. faecium SP180 is vancomycin resistant (vanA) and multiply resistant to several FDA approved antibiotics. The effects of several concentrations penicillin G on separate cultures are shown.

FIG. 20 provides a “time-killing curve” illustrating the number of surviving Eneterococus faecium SP 180 (cfu/ml) as a function of time after cells were exposed to penicillin at room temperature. E. faecium SP180 is vancomycin resistant (vanA) and multiply resistant to several FDA approved antibiotics. The effects of several concentrations of penicillin on separate cultures are shown.

FIG. 21 provides a thin section electron microscopy image of untreated S. aureus (ATCC 25923) displaying a normal intact membrane.

FIG. 22 provides a thin section electron microscopy image of S. aureus (ATCC 25923) after exposure to 2×MIC concentration of cyclo[KSKWLWLW] for 2 h. The image provides direct visualization of the membrane mode of action. Arrows denote abnormal membrane structures caused by the peptide action.

FIG. 23 provides a thin section electron microscopy image of S. aureus (ATCC 25923) after exposure to 2×MIC concentration of cyclo[KSKWVWLW] for 2 h. The image provides direct visualization of the membrane mode of action. Arrows denote abnormal membrane structures caused by the peptide action.

FIG. 24 illustrates the protective effects of the present cyclic peptides on survival of mice lethally infected with S. aureus MRSA (ATCC 33591). The mouse peritonitis model was used with intraperitoneal infection (right side) of bacteria in 5% mucin and peptide injection (IP left side) 45-60 min post infection. Survival was monitored over 14 days (death as an end point); eight mice were tested per dose. In FIG. 24a, the mice were injected with varying concentrations of cyclo[RRKWLWLW]HCl. In FIG. 24b, the mice were injected with varying concentrations of cyclo[KQRWLLWLW]HCl.

FIG. 25 illustrates the protective effects of the present cyclic peptides on survival of mice infected with S. aureus MRSA (ATCC 33591). The mouse peritonitis model was used with intraperitoneal infection (right side) of bacteria in 5% mucin and peptide injection (IP left side) 45-60 min post infection. Survival was monitored over 14 days (death as an end point); eight mice were tested per dose. In FIG. 25a, the mice were injected with varying concentrations of cyclo[KSKWLWLW]HCl. In FIG. 25b, the mice were injected with varying concentrations of cyclo[KKKWLWLW]HCl.

FIG. 26 illustrates the protective effects of the present cyclic peptides on survival of mice infected with S. aureus MRSA (ATCC 33591). The mouse peritonitis model was used with intraperitoneal infection (right side) of bacteria in 5% mucin and peptide injection (IP left side) 45-60 min post infection. Survival was monitored over 14 days (death as an end point); eight mice were tested per dose. The mice were injected with varying concentrations of cyclo[KKLWLW]HCl.

FIG. 27 illustrates the protective effects of the present cyclic peptides on survival of mice infected with vancomycin resistant E. faecium (VREF)(ATCC 51575). The mouse peritonitis model was used with intraperitoneal infection (right side) of bacteria in 5% mucin and peptide injection (IP left side) 45-60 min post infection. Survival was monitored over 14 days (death as an end point); eight mice were tested per dose. In FIG. 27a, the mice were injected with varying concentrations of cyclo[RRKWLWLW]HCl. In FIG. 27b, the mice were injected with varying concentrations of cyclo[KSKWLWLW]HCl.

FIG. 28 illustrates the protective effects of the present cyclic peptides on survival of mice infected with S. aureus MRSA (ATCC 33591). The mouse peritonitis model was used with intraperitoneal infection (right side) of bacteria in 5% mucin and peptide injection (IP left side) 45-60 min post infection. Survival was monitored over 14 days (death as an end point); four mice were tested per dose. In FIG. 28a, the mice were injected subcutaneously (upper neck compartment) with varying concentrations of cyclo[RRKWLWLW]HCl. In FIG. 28b, the mice were injected intravenously with varying concentrations of cyclo[RRKWLWLW]HCl. Peptide doses indicated are for each injection. Five doses at the indicated mg/kg quantities were administered intravenously 8-12 h apart.

FIG. 29 illustrates the toxicity of peptides administered via intraperitoneal route. In FIG. 29a, the mice were injected with varying concentrations of cyclo[RRKWLWLW]HCl. In FIG. 29b, the mice were injected with varying concentrations of cyclo[KQRWLVWLW]HCl. Mice were monitored over a period of 14 days for activity and mortality. Four mice per dose were used in each experiment.

FIG. 30 illustrates the toxicity of peptides administered via intraperitoneal route. In FIG. 30a, the mice were injected with varying concentrations of cyclo[KSKWLWLW]HCl. In FIG. 30b, the mice were injected with varying concentrations of cyclo[KUWLW]HCl. Mice were monitored over a period of 14 days for activity and mortality. Four mice per dose were used in each experiment.

### DETAILED DESCRIPTION OF THE INVENTION

The present invention provides small cyclic peptides and compositions that quickly and selectively kill microbes without substantial or undesired toxicity toward mammalian cells. The present invention includes cyclic peptides, and pharmaceutical compositions comprising cyclic peptides, with either a sequence of alternating D-, and L-α-amino acids, or a sequence of β-amino acids, that can sample flat, ring-shaped conformations. Such ring-shaped conformations project the amino acid side chains of the cyclic peptides away from the center of the ring and orient the amide backbone approximately perpendicular to the plane of the ring structure. It is believed that under conditions that favor hydrogen bonding, such as side chain charge neutualization through interactions with cell membrane constituents and/or contact with low dielectric constant environments of cell membranes, the cyclic peptides can self-assemble via intermolecular hydrogen bonding to form supramolecular structures. Cyclic peptides that simply contain one or more D-amino acids do not adopt a flat ring-shaped conformation and do not have the backbone conformation needed for self-assembly of the cyclic peptide into supramolecular structures. Target microbial organisms against which the present cyclic peptides are effective include microbes, including any single cell organism or parasite that has a cellular membrane and that can infect a mammal. For example, target microbial organisms include bacteria, fungi, helminths, protozoa, yeast strains and other single cell organisms. Cyclic peptides of the invention have been found to be active against both gram-negative and gram-positive bacteria.

Small differences in environment, for example, the difference in composition of different cellular membranes, can influence the course and nature of the proposed assembly process. This feature is used in the present invention both to target and to optimize the anti-microbial activity of selected cyclic peptides against particular microbial species, while providing substantially no toxicity, or no undesired toxicity, in mammalian cells at therapeutically effective doses and dose regimens. Supramolecular structures are believed to respond to their immediate environment through dynamic self-assembling/disassembling processes to quickly find the most thermodynamically favored assembly. It is believed that, during assembly, peptide supramolecular structures sense and respond to the environment of a cellular membrane by sampling various topologically related assemblies. In non-target mammalian membranes at therapeutically desirable concentrations, it is believed that the preferred cyclic peptides of the invention do not or cannot adopt a thermodynamically favorable supramolecular structure. Thus, mammalian membranes are not substantially or undesirably affected by the presence of such cyclic peptides. However, in selected microbial membranes the present cyclic peptides are believed to form unique energetically favorable supramolecular structures that destabilize (e.g., lyse), permeablize and/or depolarize the microbial membrane, thereby disrupting microbial transmembrane ion and electrical gradients and other vital functions, and quickly leading to microbial cell death.

Small changes in amino acid sequence of a cyclic peptide can be amplified into large differences at the supramolecular level. Thus, changes in the structure of a cyclic peptide may constrain peptide interaction and limit formation of supramolecular structures to particular cellular membranes that have particular membrane constituents, membrane partitioning properties, uptake properties, and the like.

Another feature of the present self-assembling peptide supramolecular structures is believed to be the potential for a given cyclic peptide to form a number of diastereomeric nanotube assemblies. This property stems from the fact that backbone-backbone hydrogen bonding are believed primarily to direct the self-assembly of the nanotube structure. Differently stacked subunits can give rise to topoisomeric supramolecular structures that share the same or nearly the same tubular β-sheet-like hydrogen bonded backbone structure. The variety of supramolecular structures assembled from a single cyclic peptide minimizes the probability that microbes can develop resistance to these antibiotic agents.

By varying the peptide sequence while retaining a cyclic D- and L-α-peptide backbone, or the cylic β-peptide backbone, a multitude of cyclic peptides can quickly be screened or evaluated for the ability to selectively target and assemble in microbial membranes. They can also be screened and evaluated for anti-microbial activity by testing for increased membrane permeability, depolarization, or destabilization.

The term “amino acid,” includes the residues of the natural α-amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as β-amino acids, synthetic and unnatural amino acids. Many types of amino acid residues are useful in the cyclic peptides and the invention is not limited to natural, genetically-encoded amino acids. Examples of amino acids that can be utilized in the cyclic peptide described herein can be found, for example, in Fasman, 1989, CRC Practical Handbook of Biochemistry and Molecular Biology, CRC Press, Inc., and the references cited therein. Another source of a wide variety of amino acid residues is provided by the website of RSP Amino Acids Analogues, Inc. (www.amino-acids.com).

The term “mammal,” as used herein, refers to an animal, in general, a warm-blooded animal, which is susceptible to or has a microbial infection. Mammals include cattle, buffalo, sheep, goats, pigs, horses, dogs, cats, rats, rabbits, mice, and humans. Also included are other livestock, domesticated animals and captive animals. The term “farm animals” includes chickens, turkeys, fish, and other farmed animals.

As used herein, a “nanotube” or “nanotubule” is a small tubule that may spontaneously form from the cyclic peptides of the present invention. The present cyclic peptides are believed to stack to form supramolecular structures composed of nanotubes. Hydrogen bonding between cyclic peptide is believed to help drive the self-assembly of the supramolecular structures from the cyclic peptide. Each nanotube has a pore in the center of the tube that is surrounded by the series of peptide backbones of the stacked cyclic peptides that form the nanotubes. The size of the pore depends upon the number of amino acids in the cyclic peptides that form the nanotube. In general, depending on the ring size of the cyclic peptides employed, ions, sugars, and other small molecules can travel through the pores of the nanotubes. Larger molecules can also flow through pores formed from larger cyclic peptides and supramolecular structures formed of aggregates of nanotubes. For example, in some embodiments the supramolecular structure is thought to be a barrel-like structure composed of clusters of nanotubes. In other embodiments, the supramolecular structure is thought to be a “carpet” or “carpet-like” arrangement of nanotubes.

The term “peptide” as used herein includes a sequence of from four to sixteen amino acids residues in which the α-carboxyl group of one amino acid is joined by an amide bond to the main chain (α- or β-) amino group of the adjacent amino acid. The peptides provided herein for use in the described and claimed methods and compositions are cyclic. Peptide sequences specifically recited herein are written with the amino terminus on the left and the carboxy terminus on the right. However, where the peptides are shown in cyclic form, the first amino acid in the sequence is arbitrarily chosen. Moreover, for formulae of cyclic peptides where the sequence extends onto two lines, the sequence on the second line extends from the N-terminal side on the right to the C-terminal side on the left.

According to the present invention, “supramolecular structures” are multi-subunit structures, e.g. nanotubes, barrels and carpets of nantubules, which are believed to be formed through “noncovalent” assembly of cyclic peptides. Supramolecular structures may be contrasted with molecular or polymeric systems in which the product of covalent bond formation between reactants or monomers. The proposed peptide supramolecular structures are thermodynamically controlled assemblies that can undergo reversible structural assembly and disassembly. Such assembly-disassembly will depend, for example, on the environment, subunit structure, side group selection, side group interaction, and the nature and combination of noncovalent forces operating on the system. In contrast, covalent polymeric structures have been used to design kinetically stable structures rather than structures that assemble and dissemble in response to the environment. Hence, one attractive feature of the present compositions containing peptides that can form supramolecular structures is their ability to select amongst various cell membrane types. Such selection is driven by favorable thermodynamic forces determined by the composition of the cyclic peptide relative to the cell membrane environment and the molecular and/or supramolecular constituents of the cell membrane.

The term “substantially no” with reference to self-assembly, hemolysis, toxicity or cellular lysis, or the like, means that little or no self-assembly, hemolysis, toxicity, cellular lysis or the like is present at the tested or desired peptide dosage or concentration. By way of example, “substantially no” hemolysis can mean that less than about 20%, alternatively less than 15% or less than 10%, or no detectable, hemolysis at the tested or desired peptide dosage or concentration has occurred. Similarly, “substantially no” toxicity or lysis can mean that less than about 20%, alternatively less than 15% or less than 10%, or no detectable, toxicity or lysis at the tested or desired peptide dosage or concentration has occurred. In other embodiments, “substantially no” hemolysis, toxicity or lysis means that less than about 5%, or no detectable, hemolysis, toxicity or lysis, etc., at the tested or desired peptide dosage or concentration has occurred.

The term “therapeutically effective amount” is that amount sufficient to control a microbial infection. A therapeutically effective amount generally controls the amount of microbes and/or a disease state characterized by the presence of microbes in the infected mammal, for example, by at least about 20%, by at least about 40%, by at least about 60%, or by at least about 80% relative to untreated subjects. In some embodiments, a therapeutically effective amount controls the amount of microbes and/or a disease state characterized by the presence of microbes in the infected mammal by at least about 90% or more. These percentages refer to a decrease in the amount of microbes found in the infected mammal and/or the decrease in symptoms associated with a disease state characterized by the presence of microbes in the infected mammal relative to untreated subjects. In other embodiments, a “therapeutically effective amount” is that amount of cyclic peptide needed to permeabilize, depolarize or destabilize the cellular membrane of the target microbial organism causing the infection. The term “therapeutically effective amount” can also be the amount needed to kill the target microbial organisms causing the infection. An effective amount of the therapeutic agent necessary to control microbes can vary according to factors such as the type of microbes, the amount of microbes already present in the animal, the age, sex, health and weight of the mammal, and the ability of the cyclic peptides of the present invention to control microbial infections in the mammal.

Therapeutically effective amounts of the peptide and peptide compositions can also be used to prevent microbial infection, including preventing a recurrence of microbial infection.

Peptides, Peptide Variants, and Derivatives Thereof

The present invention provides cyclic peptides and compositions including cyclic peptides that have an amino acid sequence of alternating D- and L-amino acids that is between four to about sixteen, alternatively about six to about sixteen amino acids in length. Alternatively, the cyclic peptides of the present invention can have between three to about ten β-amino acids. In general, the cyclic D, L-α-peptides do not include the amino acids proline and glycine. According to the invention, β-amino acids can be substituted at the α- or β-carbons, or both. Mono-substituted β-amino acids of either S or R chirality can be employed for the construction of cyclic β-peptides, provided that the cyclic beta peptide is homochiral. Disubstituted β-amino acids employed in the present invention must have the relative R,R or S,S diastereomeric configuration, provided that the β-amino acid residues in a cyclic peptide structure are homochiral. Cyclic peptides having β-amino acids generally have at least one β-amino acid with at least one polar side chain.

The cyclic peptides of the present invention are believed to undergo self-assembly to form supramolecular structures that, upon assembly in or on a microbial membrane, can cause depolarization and/or permeablization and/or destailization of the microbial membrane. In some cases, the cyclic peptides cause lysis of the microbe, e.g. a bacterium. Self-assembly into supramolecular structures is thought to occur by stacking of the cyclic peptides in an anti-parallel fashion or a parallel fashion with formation of β-sheet hydrogen bonds between adjacent cyclic peptides. However, it is believed that the preferred cyclic peptides do not readily self-assemble into supramolecular structures in mammalian cellular membranes as measured, for example, in an assay for toxicity in mammalian cells or hemolysis of mammalian red blood cells at tested or therapeutically effective doses.

Cyclic peptides of the present invention can be made from α-amino acids or β-amino acids. The amino acid sequence of the present cyclic peptides includes at least one polar amino acid in the case of D,L α-amino acid cyclic peptides, or at least one polar side chain in the case of cyclic β-peptides. The percentage of polar amino acids can range, for example, from about 25% or 33% to about 65% or 88%. However, in some embodiments a majority of the amino acids are polar. For example, the percentage of polar amino acids can be from about 50% to about 88% of the total number of amino acids. The exact number of polar and nonpolar amino acids depends on the size and the properties sought for a given cyclic peptide. In some embodiments, the sizes for the present cyclic peptides are about six to about ten D,L α-amino acids or three to about ten β-amino acids. In other embodiments, the size for the present cyclic peptides is about six to about eight D,L α-amino acids or four to about six β-amino acids. Thus, for example, an eight residue cyclic peptide of the invention can have at least one, alternatively, two to seven polar D- and/or L-α-amino acids. Other eight residue cyclic peptides will have three to five polar D- and/or L-α-amino acids for example. Preferred eight residue cyclic peptides have three, four or five polar amino acids. In some embodiments, for example, six residue cyclic peptides of the invention can have two to five polar D- and/or L-α-amino acids. Other six residue cyclic peptides may have three to four polar D- and/or L-α-amino acids. At least one of these polar D- or L-α-amino acids may be adjacent to at least one other polar D- or L-α-amino acid. Alternatively, at least one polar D- or L-α-amino acid may be adjacent only to nonpolar D- or L-α-amino acids. Beta peptides having about four to about eight β-amino acids may have, for example, about two to twelve polar side chains, depending on the level of α and β backbone substitution.

The cyclic D- L-α-peptides of the invention generally have about 25% to about 88% ionizable amino acid residues. In some embodiments, the percentage of ionizable amino acids can be from about 33% or 50% to about 65% or 88% of the total number of D- and/or L-amino acids. Thus, for example, a six or eight residue cyclic peptide can have at least one, or alternatively two or three or more ionizable D- and/or L-amino acids. In other embodiments, the cyclic peptides of the invention can have four to six ionizable D- and/or L-amino acids. Such an ionizable D- or L-amino acid can be adjacent to at least one other polar or ionizable D- or L-amino acid. Alternatively, the cyclic peptides of the invention can have at least one ionizable D- or L-amino acid that is adjacent only to nonpolar D- or L-amino acids. The cyclic β-peptides of the invention generally have about 25% to about 88% ionizable amino acid side chains. In some embodiments, the percentage of ionizable amino acid side chains can be from about 33% or 50% to about 65% or 88% of the total number of amino acid side chains. Thus, for example, a four to six residue cyclic β-peptide can have at least one, or alternatively two or three or more ionizable amino acid side chains. In other embodiments, the cyclic β-peptides of the invention can have four to six ionizable amino acid side chains.

The cyclic peptides of the invention can have nonpolar D- and/or L-amino acid residues. The number of non-polar amino acids chosen can vary as the size of the peptide varies and as the selected microbial membrane environment varies. The cyclic peptides of the invention generally have about 12% to about 75% D- and L-nonpolar amino acids. In some embodiments, the percentage of nonpolar amino acids can be from about 50% to about 67% or 75% of the total number of D- and L-amino acids. Thus, for example, an eight residue cyclic peptide of the invention can have at least one, alternatively, two to seven nonpolar D- and/or L-amino acids. Other eight residue cyclic peptides may have three to five nonpolar D- and/or L-amino acids. In some embodiments, for example, six residue cyclic peptides of the invention have two to five nonpolar D- and/or L-amino acids. Other six residue cyclic peptides may have three to four nonpolar D- and/or L-amino acids. At least one of these nonpolar D- or L-amino acids may be adjacent to at least one other nonpolar D- or L-amino acid. Alternatively, at least one nonpolar D- or L-amino acid may be adjacent only to polar D- or L-amino acids. In general, the cyclic peptides do not include the amino acid proline or glycine, but certain cyclic peptides may have good activity even though proline or glycine is included.

According to the invention, β-amino acids can have non-polar side chains at the α- or β-carbons, or both. The number of non-polar amino acid side chains chosen can vary as the size of the peptide varies and as the selected microbial membrane environment varies. The cyclic β-peptides of the invention generally have about 12% to about 75% nonpolar amino acid side chains. In some embodiments, the percentage of nonpolar amino acid side chains can be from about 50% to about 67% or 75% of the total number of amino acid side chains. Thus, for example, an eight residue cyclic β-peptide of the invention can have at least one, alternatively, two to seven nonpolar amino acid side chains. Other eight residue cyclic β-peptides may have three to five nonpolar amino acid side chains. In some embodiments, for example, six residue cyclic β-peptides of the invention have two to five nonpolar amino acid side chains. Other six residue cyclic β-peptides may have three to four nonpolar amino acid side chains.

Amino acids used in the cyclic peptides can be genetically encoded amino acids, naturally occurring non-genetically encoded amino acids, or synthetic amino acids. Both L- and D-enantiomers of any of the above are utilized in the cyclic peptides. The amino acid notations used herein for the twenty genetically encoded L-amino acids and some examples of non-encoded amino acids are provided in Table 1.

TABLE 1

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| B-Alanine | | Bala |
| 2,3-Diaminopropionic | | Dpr |
| acid | | |
| A-Aminoisobutyric acid | | Aib |
| N-Methylglycine | | MeGly |
| (sarcosine) | | |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3-carboxylic | | |
| acid | | |
| B-2-thienylalanine | | Thi |
| Methionine sulfoxide | | MSO |
| Homoarginine | | Harg |
| N-acetyl lysine | | AcLys |
| 2,4-Diamino butyric acid | | Dbu |
| P-Aminophenylalanine | | Phe(pNH₂) |
| N-methylvaline | | MeVal |
| Homocysteine | | Hcys |
| Homoserine | | Hser |
| E-Amino hexanoic acid | | Aha |
| Δ-Amino valeric acid | | Ava |
| 2,3-Diaminobutyric acid | | Dab |

Certain commonly encountered amino acids that are not genetically encoded and that can be present in the cyclic peptides of the invention include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); methylglycine (MeGly); ornithine (Orn); citrulline (Cit); t-butylalanine (t-BuA); t-butylglycine (t-BuG); N-methylisoleucine (Mehle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). Additional amino acid analogs contemplated include phosphoserine, phosphothreonine, phosphotyrosine, hydroxyproline, gamma-carboxyglutamate, hippuric acid, octahydroindole-2-carboxylic acid, statine, α-methyl-alanine, para-benzoyl-phenylalanine, propargylglycine, and sarcosine. Peptides that are encompassed within the scope of the invention can have any of foregoing amino acids in the L- or D-configuration, or any other amino acid known to one of skill in the art.

Amino acids that are substitutable for each other generally reside within similar classes or subclasses. As known to one of skill in the art, amino acids can be placed into different classes depending primarily upon the chemical and physical properties of the amino acid side chain. For example, some amino acids are generally considered to be hydrophilic or polar amino acids and others are considered to be hydrophobic or nonpolar amino acids. Polar amino acids include amino acids having acidic, basic or hydrophilic side chains and nonpolar amino acids include amino acids having aromatic or hydrophobic side chains. Nonpolar amino acids may be further subdivided to include, among others, aliphatic amino acids. The definitions of the classes of amino acids as used herein are as follows:

“Nonpolar Amino Acid” refers to an amino acid having a side chain that is uncharged at physiological pH, that is not polar and that is generally repelled by aqueous solution. Examples of genetically encoded hydrophobic amino acids include Ala, Ile, Leu, Met, Trp, Tyr and Val. Examples of non-genetically encoded nonpolar amino acids include t-BuA, Cha and Nle.

“Aromatic Amino Acid” refers to a nonpolar amino acid having a side chain containing at least one ring having a conjugated π-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, (3-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Aliphatic Amino Acid” refers to a nonpolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Polar Amino Acid” refers to a hydrophilic amino acid having a side chain that is charged or uncharged at physiological pH and that has a bond in which the pair of electrons shared in common by two atoms is held more closely by one of the atoms. Polar amino acids are generally hydrophilic, meaning that they have an amino acid having a side chain that is attracted by aqueous solution. Examples of genetically encoded polar amino acids include asparagine, cysteine, glutamine, lysine and serine. Examples of non-genetically encoded polar amino acids include citrulline, homocysteine, N-acetyl lysine and methionine sulfoxide.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Ionizable Amino Acid” refers to an amino acid that can be charged at a physiological pH. Such ionizable amino acids include acidic and basic amino acids, for example, D-aspartic acid, D-glutamic acid, D-histidine, D-arginine, D-lysine, D-hydroxylysine, D-ornithine, L-aspartic acid, L-glutamic acid, L-histidine, L-arginine, L-lysine, L-hydroxylysine or L-ornithine.

As will be appreciated by those having skill in the art, the above classifications are not absolute. Several amino acids exhibit more than one characteristic property, and can therefore be included in more than one category. For example, tyrosine has both a nonpolar aromatic ring and a polar hydroxyl group. Thus, tyrosine has several characteristics that could be described as nonpolar, aromatic and polar. However, the nonpolar ring is dominant and so tyrosine is generally considered to be nonpolar. Similarly, in addition to being able to form disulfide linkages, cysteine also has nonpolar character. Thus, while not strictly classified as a hydrophobic or nonpolar amino acid, in many instances cysteine can be used to confer hydrophobicity or nonpolarity to a peptide.

The classifications of the above-described genetically encoded and non-encoded amino acids are summarized in Table 2, below. It is to be understood that Table 2 is for illustrative purposes only and does not purport to be an exhaustive list of amino acid residues that may comprise the peptides and peptide analogues described herein. Other amino acid residues that are useful for making the peptides described herein can be found, e.g., in Fasman, 1989, CRC Practical Handbook of Biochemistry and Molecular Biology, CRC Press, Inc., and the references cited therein. Another source of amino acid residues is provided by the website of RSP Amino Acids Analogues, Inc. (www.amino-acids.com). Amino acids not specifically mentioned herein can be conveniently classified into the above-described categories on the basis of known behavior and/or their characteristic chemical and/or physical properties as compared with amino acids specifically identified.

TABLE 2

| Classification | Genetically Encoded | Genetically Non-Encoded |
|---|---|---|
| Nonpolar | | |
| Aromatic | F, Y, W | Phg, Nal, Thi, Tic, Phe(4- |
| | | Cl), Phe(2-F), Phe(3-F), |
| | | Phe(4-F), Pyridyl Ala, |
| | | Benzothienyl Ala |
| Aliphatic | A, V, L, I | t-BuA, t-BuG, MeIle, Nle, |
| | | MeVal, Cha, bAla, MeGly, |
| | | Aib |
| Other | M, G, P | |
| Nonpolar | | |
| Polar | | |
| Acidic | D, E | |
| Basic | H, K, R | Dpr, Orn, hArg, Phe(p- |
| | | NH₂), DBU, A₂ BU |
| Neutral Polar | S, T, Y, Q, N, D, E, H, | Cit, AcLys, MSO, hSer, |
| | R, K, C | Orn, Hcys |
| Cysteine-Like | C | Pen, hCys, β-methyl Cys |

In some embodiments, polar amino acids contemplated by the present invention include, for example, arginine, asparagine, aspartic acid, cysteine, glutamic acid, glutamine, histidine, homocysteine, lysine, hydroxylysine, ornithine, serine, threonine, the corresponding β-amino acids, and structurally related amino acids. In one embodiment the polar amino is an ionizable amino acid such as arginine, aspartic acid, glutamic acid, histidine, hydroxylysine, lysine, or ornithine.

Examples of nonpolar or nonpolar amino acid residues that can be utilized include, for example, alanine, valine, leucine, methionine, isoleucine, phenylalanine, tryptophan, tyrosine and the like.

In addition, the amino acid sequence of a peptide can be modified so as to result in a peptide variant that includes the substitution of at least one amino acid residue in the peptide for another amino acid residue, including substitutions that utilize the D rather than L form.

One or more of the residues of the peptide can be exchanged for another, to alter, enhance or preserve the biological activity of the peptide. Such a variant can have, for example, at least about 10% of the biological activity of the corresponding non-variant peptide. Conservative amino acid substitutions are often utilized, i.e., substitutions of amino acids with similar chemical and physical properties, as described above.

Hence, for example, conservative amino acids substitutions involve exchanging aspartic acid for glutamic acid; exchanging lysine for arginine or histidine; exchanging one nonpolar amino acid (alanine, isoleucine, leucine, methionine, phenylalanine, tryptophan, tyrosine, valine,) for another; and exchanging one polar amino acid (aspartic acid, asparagine, glutamic acid, glutamine, glycine, serine, threonine, etc.) for another. After the substitutions are introduced, the variants are screened for biological activity.

In one embodiment, the cyclic peptides of the invention can have an amino acid sequence having formula I: 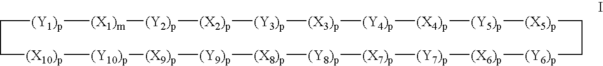
wherein:
   m is an integer ranging from 1 to 7;
   each p is separately an integer ranging from 0 to 7;
   each X₁, X₂, X₃, X₄, X₅, X₆, X₇, X₈, X₉, and X₁₀ is separately a polar D- or L-α-amino acid; and
   each Y₁, Y₂, Y₃, Y₄, Y₅, Y₆, Y₇, Y₈, Y₉, and Y₁₀ is separately nonpolar D- or L-α-amino acid; and
wherein the cyclic peptide has an even number of from four to about sixteen alternating D- and L-α amino acids.

In another embodiment, the cyclic peptides of the invention can have an amino acid sequence having formula II: 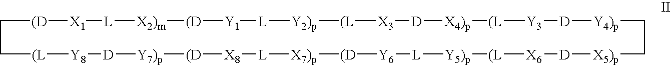
wherein:
   m is an integer ranging from 1 to 7;
   each p is separately an integer ranging from 0 to 7;
   each X₁, X₂, X₃, X₄, X₅, X₆, X₇, and X₈ is separately a polar D- or L-α-amino acid;
each Y₁, Y₂, Y₃, Y₄, Y₅, Y₆, Y₇, and Y₈ is separately nonpolar D- or L-α-amino acid; and
wherein the cyclic peptide has an even number of from four to about sixteen alternating D- and L-α amino acids.

In yet another embodiment, the cyclic peptides of the invention can have an amino acid sequence having formula III: 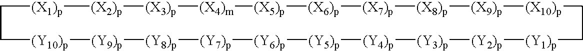
III.
wherein:
   m is an integer ranging from 1 to 7;
   each p is separately an integer ranging from 0 to 7;
   each X₁, X₂, X₃, X₄, X₅, X₆, X₇, X₈, X₉, and X₁₀ is separately a polar D- or L-α-amino acid;
   each Y₁, Y₂, Y₃, Y₄, Y₅, Y₆, Y₇, Y₈, Y₉, and Y₁₀ is separately nonpolar D- or L-α-amino acid; and
wherein the cyclic peptide has an even number of from four to about sixteen alternating D- and L-α amino acids.

In yet another embodiment, the cyclic peptide has an amino acid sequence of formula IVa or IVb: 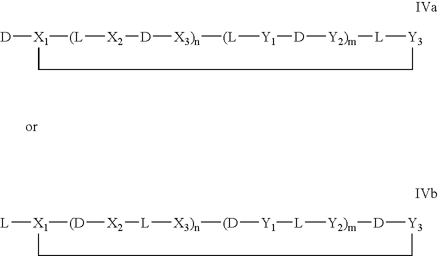
wherein:
   n is an integer ranging from 0 to 4;
   m is an integer ranging from 1 to 7;
   X₁, X₂ and X₃ are each a separate a polar amino acid;
   Y₁, Y₂ and Y₃ are each a separate nonpolar amino acid; and
wherein the cyclic peptide has an even number of from four to about sixteen alternating D- and L-α amino acids.

In another embodiment, the cyclic peptide has an amino acid sequence of formula Va or Va: 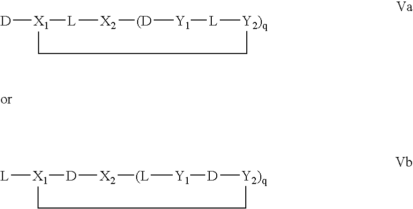
wherein:
   q is an integer ranging from 2 to 7;
   X₁ and X₂ are separately polar amino acids;
   Y₁ and Y₂ are separately nonpolar amino acids.

The claimed cyclic peptides of the above formulae, however, exclude those composed entirely of nonpolar amino acids. Moreover, a peptide having any one of the following sequences may be excluded from one or more of the above formulae:

cyclo-[-(Gln-D-Ala)₄-]
cyclo-[-(Gln-D-Leu)₄-]
cyclo-[-(Gln-D-Val)₄-]
cyclo-[-(Phe-D-Leu)₄-]
cyclo-[-(Phe-D-Ala)₄-]
cyclo-[-(Gln-D-Ala-Glu-D-Ala)₂-]
cyclo-[-(Gln-D-Phe-Glu-D-Phe)₂-]
cyclo-[-(Gln-D-Ala-Glu-D-Ala)₃-]
cyclo-[-(Trp-D-Leu)₃-Gln-D-Leu-]
cyclo-[-Gln-(D-Leu-Trp)₄-D-Leu-]
cyclo-[-Lys-D-Leu-Lys-D-Leu-Lys-D-Leu-Lys-D-Leu];
cyclo-[-Arg-D-Leu-Arg-D-Leu-Arg-D-Leu-Arg-D-Leu-];
cyclo-[-Glu-D-Leu-Glu-D-Leu-Glu-D-Leu-Glu-D-Leu-].

Additionally, the following sequences may or may not be excluded from the above formulae and associated peptides and compositions:

cyclo-[-D-Arg-Gln-D-Arg-Ala-D-Trp-Leu-D-Trp-Trp-];
cyclo-[-D-Arg-Gln-D-Arg-Leu-D-Trp-Leu-D-Trp-Trp-];
cyclo-[-D-Arg-Gln-D-Arg-Val-D-Trp-Leu-D-Trp-Trp-];
cyclo-[-D-Arg-Gln-D-Arg-Phe-D-Trp-Leu-D-Trp-Trp-];
or
cyclo-[-D-Arg-Gln-D-Arg-Trp-D-Trp-Leu-D-Trp-Trp-].

Cyclic peptides having any of the above sequences, however, including any of the excluded sequences, may be included within or excluded from pharmaceutical compositions and peptides of the present invention.

For example, the X amino acids in the above formulae can be D-serine, D-threonine, D-asparagine, D-glutamine, D-aspartic acid, D-cysteine, D-glutamic acid, D-histidine, D-homocysteine, D-arginine, D-lysine, D-hydroxylysine, D-ornithine, L-serine, L-threonine, L-asparagine, L-glutamine, L-aspartic acid, L-cysteine, L-glutamic acid, L-histidine, L-homocysteine, L-arginine, L-lysine, L-hydroxylysine or L-ornithine, provided that the a-cyclic peptide has a sequence of alternating D- and L-ac-amino acids.

In some embodiments, one or more of the X amino acids are ionizable amino acids. Such ionizable amino acids include, for example, D-aspartic acid, D-glutamic acid, D-histidine, D-arginine, D-lysine, D-hydroxylysine, D-ornithine, L-aspartic acid, L-glutamic acid, L-histidine, L-arginine, L-lysine, L-hydroxylysine or L-ornithine.

The Y amino acids in the above formulae can be, for example, L-alanine, L-valine, L-leucine, L-methionine, L-isoleucine, L-phenylalanine, L-tryptophan, L-tyrosine, D-alanine, D-valine, D-leucine, D-methionine, D-isoleucine, D-phenylalanine, D-tyrosine or D-tryptophan, provided that the a-cyclic peptide has a sequence of alternating D- and L-α-amino acids. In other embodiments, the Y amino acids may be L-tryptophan, D-tryptophan, L-leucine or D-leucine, provided that the cyclic peptide has a sequence of alternating D- and L-amino acids.

The cyclic peptides of the present invention, for example, include any of SEQ ID NO:5, 7-22, 26-29, 40, 41, 43-55, 57, 58, 61-67, 72-77, 79-89, 91-93, 97-102, 107, 109-112, 114-117, 119-122, 125, 126, 128, 129, 133, 139, 140 or 141. In some embodiments the cyclic peptides employed are those with SEQ ID NO:8, 9, 12, 17, 18, 26, 29, 47-52, 61, 63, 67, 68, 72-77, 84, 85, 87-89, 91-93, 100, 102, 107, 111, 112, 119, 125 and 139. Formulations or compositions containing the present cyclic peptides can include a mixture of two or more cyclic peptides.

The present isolated, purified peptides or variants thereof, can be synthesized in vitro, e.g., by the solid phase peptide synthetic method or by enzyme catalyzed peptide synthesis or with the aid of recombinant DNA technology. Solid phase peptide synthetic method is an established and widely used method, which is described in references such as the following: Stewart et al., Solid Phase Peptide Synthesis, W. H. Freeman Co., San Francisco (1969); Merrifield, J. Am. Chem. Soc. 85 2149 (1963); Meienhofer in “Hormonal Proteins and Peptides,” ed.; C. H. Li, Vol.2 (Academic Press, 1973), pp.48-267; and Bavaay and Merrifield, “The Peptides,” eds. E. Gross and F. Meienhofer, Vol.2 (Academic Press, 1980) pp.3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

To identify highly active cyclic peptides that have little or no undesired toxicity for mammalian cells, individual cyclic peptides, or libraries of cyclic peptides can be made and the individual cyclic peptides or cyclic peptides from those libraries can be screened for anti-microbial activity and toxicity. For example, libraries of peptides can be made using a one-bead-one-compound strategy provided by Lam et al. (97 Chem. Rev. 411-448 (1997) or synthesized on macrobeads by a split and pool method of Furka, et al. (37 Int. J. Pept. Prot. Res. 487-493(1991)). Mass spectrometric sequence analysis techniques enable rapid identification of every peptide within a given library. See, Biemann, K. 193 Methods Enzymol. 455 (1990). In general, synthetic operations, including peptide cyclization, are performed on solid support to avoid laborious and difficult to automate solution-phase operations. Moreover, the final product of the synthesis regimen is generally sufficiently pure for biological assays without laborious purification procedures. Peptide yields from each synthesis can be sufficient for performing 50 to 100 assays. Rapid, automatable mass-spectrometry-based peptide sequence analysis can be performed to identify peptide sequences that have high activity and to discard peptide sequences with low activity.

The synthetic approach employed can provide individually separable and identifiable peptide sequences to avoid the use of combinatorial library mixtures and laborious deconvolution techniques. However, libraries of impure mixtures of peptides can also be generated for testing. Impure preparations of peptides can be used for quick screening of combinations of sequences. When a mixture of peptides shows activity, the peptides in the mixture can either be individually isolated and tested or pure peptides having sequences known to be present in the impure mixture can be individually prepared and tested.

Salts of carboxyl groups of a peptide or peptide variant of the invention may be prepared in the usual manner by contacting the peptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide or peptide variants may be prepared by utilizing an N-acyl protected amino acid for the final condensation, or by acylating a protected or unprotected peptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N-acylation and O-acylation may be carried out together, if desired.

Acid addition salts of the peptide or variant peptide, or of amino residues of the peptide or variant peptide, may be prepared by contacting the peptide or amine with one or more equivalents of the desired inorganic or organic acid, such as, for example, hydrochloric acid. Esters of carboxyl groups of the peptides may also be prepared by any of the usual methods known in the art.

The invention also contemplates cyclic peptides composed of one or more β amino acids. Such β-amino acids can be substituted along their peptide backbones by one to two substitutents. Such substituents can include cylcoalkyl, cycloalkenyl, and heterocylic rings that encompass the α and β carbons of the β-peptide backbone. These rings can be, for example, C₃ -C₈ cycloalkyl, cycloalkenyl or heterocyclic rings having one or more nitrogen atoms as the sole heteroatom, and can be substituted or unsubsituted. The substituents on the ring or on the α and β, carbons of the β-peptide can be, for example, hydroxy, linear or branched C₁-C₆-alkyl, alkenyl, alkynyl; hydroxy-C₁-C₆-alkyl; amino-C₁-C₆-alkyl; C₁-C₆ -alkyloxy, C₁-C₆-alkoxy-alkyl; C₁-C₆-amino; mono- or di-C₁-C₆-alkylamino; carboxamido; carboxamido-C₁-C₆-alkyl; sulfonamido; sulfonamido-C₁-C₆-alkyl, urea, cyano, fluoro, thio; C₁-C₆-alkylthio; mono- or bicyclic aryl; mono- or bicyclic heteraryl having up to 5 heteroatoms selected from N, O, and S; mono- or bicyclic aryl-C₁-C₆ and heteroaryl-C₁-C₆-alkyl and the like.

In one embodiment, the cyclic β-peptides of the invention can have an amino acid sequence of formula VI: 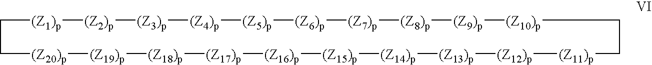
wherein:
   each p is separately an integer ranging from 0 to 7;
   each Z₁,Z₃, Z₅, Z₇, Z₉, Z₁₁, Z₁₃, Z₁₅, Z₁₇, and Z₁₉ is separately a monosubstituted β-amino acid;
   each Z₂, Z₄, Z₆, Z₈, Z₁₀, Z₁₂, Z₁₄, Z₁₆, Z₁₈, and Z₂₀ is separately a disubstituted β-amino acid; and
wherein the cyclic β-peptide has a sequence of from three to about ten homochiral β-amino acids.
   Supramolecular Structures

According to the present invention the cyclic peptides provided herein are believed self-assemble into supramolecular structures. Self-assembly means that a collection of cyclic peptides can associate to form a supramolecular structure on or within a cellular membrane without the assistance of anything other than the components of the cellular membrane. In general, the physical and chemical properties of the cellular membrane facilitate self-assembly of the cyclic peptides. The interaction between the components of microbial cellular membranes and the cyclic peptides determines whether the cyclic peptides are selective for those cellular membranes.

Formation of supramolecular structures by the peptides of the invention is supported by high-resolution imaging using cryo-electron microscopy, electron diffraction, Fourier-transform infrared spectroscopy, and molecular modeling. Supramolecular structures have been further characterized by IR spectroscopy, low-dose electron microscopy, and the analysis of electron diffraction patterns.

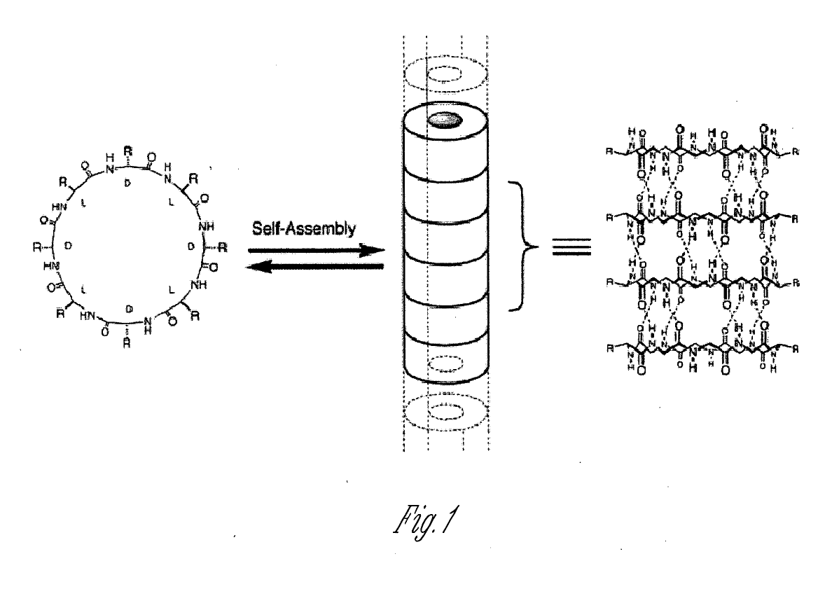

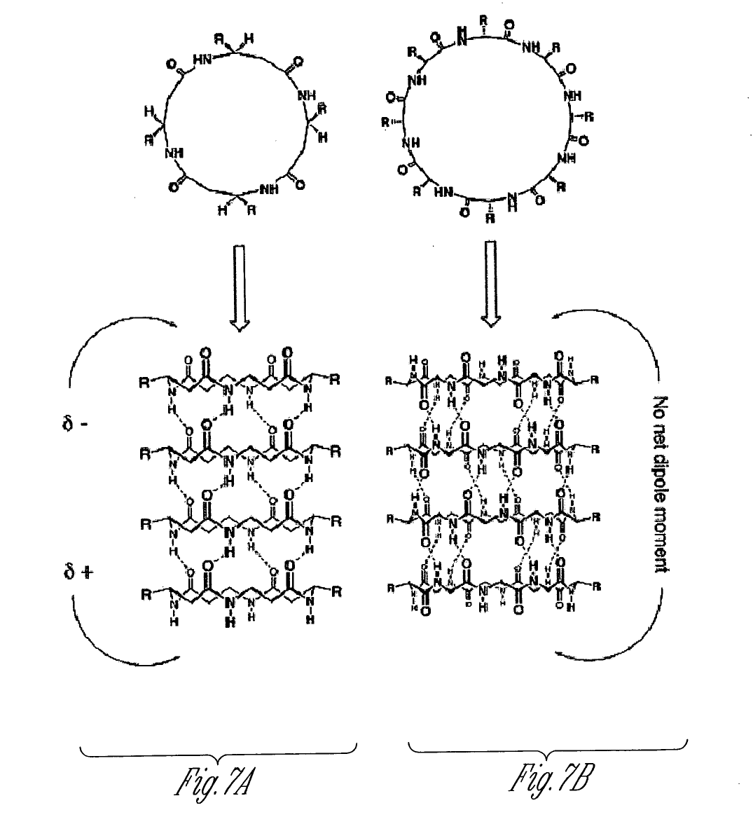

According to the present invention, cyclic peptide structures that are made up of an even number of alternating D- and L-amino acid residues are believed to adopt or sample a flat ring-shaped conformation in which all backbone amide functionalities lie approximately perpendicular to the plane of the ring structure. Similarly, cyclic peptides made up of β-amino acids can also adopt a flat-ring structure. In this flat-ring conformation, it is believed that the peptide subunits can stack, under favorable conditions, to furnish a contiguous hydrogen bonded hollow tubular structure that is referred to herein as a nanotube (see FIGS. 1 and 7).

For example, controlled acidification of alkaline solutions of peptide SEQ ID NO: I (cyclo[-(Gln-D-Ala-Glu D-Ala)₃-]) yielded rod shaped crystalline materials that appeared under transmission electron microscopy as organized bundles of tightly packed tubular structures. Low dose cryo microscopy, according to the method of M. Adrian et al. (308 Nature 32-36 (1984)) and of R. A. Milligan et al. (13 Ultramicroscopy 1-10 (1984)) revealed longitudinal striations with spacing of approximately 25 Å as expected for the center to center spacing for closely packed tubular structures. Electron diffraction patterns display axial spacing of 4.80 Å that is in agreement with the peptide stacking and the formation of tight network of hydrogen bonded b-sheet type structure. The meridonial spacing in the electron diffraction patterns display spacing of 12.67±0.06 Å and 21.94±0.05 Å characteristic of a hexagonal body centered packing of nanotubes. Hexagonal lattice resulting from the close packing of cylinders of radius r displays the characteristic two principle lattice planes of radius r and r such as the one observed here (r=12.67 Å and r=21.94 Å). The periodicity in this packing produces diffraction spots at 1/r, 2/r, and so on, and at 1/r, and 2/r, and so on. The observed electron diffraction patterns on the meridional axes extend to third order reflections (4.1 Å) signifying the ordered and crystalline state of nanotube structures. The diffraction patterns also showed a unit cell with an angle of 99° and no other symmetry than the center of symmetry pursuant to Friedel's law.

A three-dimensional supramolecular structure model was built using the parameters obtained from the electron diffraction patterns-unit cell with a=9.6 Å (2×4.80 Å for the antiparallel dimer), b=c=25.66 Å (2×12.67÷Cos9), α=120°, and β=γ99°. The model shows structure factors similar to the patterns observed in the electron diffraction thus supporting the proposed three-dimensional model. Involvement of intermolecular hydrogen bonding network in a tube-like assembly is also supported by FT-IR spectroscopic analysis according to the method of S. Krimm et al. (Advances in Protein Chemistry; Anfinsen, C. B., Edsall, J. T.; Richards, F. M. Eds.; Academic Press: Orlando, 1986, pages 181-364). Nanotubes display characteristic IR features of a β-sheet structure signified not only by the amide I bands at 1626 cm⁻¹ and 1674 cm⁻¹ and an amide II band at 1526 cm⁻¹, but also by the observed NH stretching frequency at 3291 cm⁻¹ supporting formation of a tight network of hydrogen bonds.

The IR spectrum is similar to tubular structures that have been discovered in nature. For example, nanotubular structures for some of the present peptides can be conceptually related to the structure of crystalline linear Gramicidin A that is known to form dimeric β-helical structures. Gramicidin A has amide I bands at 1630, 1685 cm⁻¹, an amide II band at 1539 cm⁻¹, and an NH stretching frequency at 3285 cm⁻¹. (V. M. Naik et al. in Biophys. J. (1986), vol. 49, pages 1147-1154.) The observed frequency of NH stretching mode correlates to an average intersubunit distance of 4.76 Å that is in close agreement with the value of 4.80 Å obtained independently from the electron diffraction patterns.

The pore size, or internal diameter, of self-assembled nanotubes can be adjusted by the ring size of the peptide subunit employed. A twelve-residue cyclic peptide structure, for example cyclo[-(Gln-D-Ala-Glu-D-Ala)₃-] (SEQ ID NO:1), has a diameter of about 13 Å. The eight residue cyclic peptide cyclo[-(Trp-D-Leu)₃-Gln-D-Leu-] (SEQ ID NO:2) has a diameter of approximately 7.5 Å in diameter. A cyclic peptide having SEQ ID NO:2 in synthetic phosphatidylcholine liposomes displays an FTIR amide-I band at 1624 cm⁻¹ and an observed N—H stretching frequency at 3272 cm⁻¹ that support formation of a tight network of β-sheet-like hydrogen bonds with an average intersubunit distance of 4.7 Å to 4.8 Å.

The flat, ring-shaped cyclic peptides of the present invention are not only structurally predisposed toward intermolecular interaction, but are also energetically favored to self-assemble on selected microbial cell membranes and permeabilize cells through formation of pores or other membrane destabilizing structures.

Formation of supramolecular structures that can permeabilize membranes was also inferred from proton transport activity. Vesicles were prepared having pH 6.5 inside and pH 5.5 in the outside bulk solution. The collapse of the imposed pH gradient in these vesicles, upon formation of the putative transmembrane channel structure, was studied by monitoring the fluorescence intensity of an entrapped pH-sensitive dye. (V. E. Carmichael et al, in J.Am. Chem. Soc. (1989), vol. 111, pages 767-769). Addition of peptide cyclo[-(Trp-D-Leu)₃-Gln-D-Leu-] (SEQ ID NO:2) to such vesicles suspensions causes a rapid collapse of the pH gradient.

Unilamellar vesicles were prepared by the reverse-phase evaporation using DPPC, OPPC, cholesterol in the ratio of 1:1:2 in a solution containing 5(6)-carboxyfluorescein (20 mM in phosphate/saline buffer: 137 mM NaCl, 2.6 mM KCI, 6.4 mM Na₂HPO₄, 1.4 mM KH₂PO₄, pH 6.5) according to the method of F. Szoka et al. in Proc. Natl. Acad. Sci. USA (1978), vol. 75, pages 4194-4198. Liposomes were then sized by multiple extrusions through Nucleopore® polycarbonate membranes (10 times, 50 psi, using 0.8 and 2×0.4 micron filter stacks) and the untrapped 5(6)-carboxyfluorescein was removed by size exclusion chromatography (Sephadex G-25 column 1×30 cm) using the same phosphate/saline buffer according to the method of F. Olson et al. in Biochim. Biophys. Acta (1979), vol. 557, pages 9-23. Vesicles formed in this way are approximately 150 nanometer in diameter as determined by electron microscopy. (R. R. C. New, Ed. Liposomes, Oxford university Press, 1990). In each experiment, 70 ml of the stock vesicle solution (3.5×10⁻³ M in phospholipids) was added to pH 5.5 buffer (1.3 ml, 137 mM NaCl, 2.6 mM KCl, 6.4 Na₂HPO₄, 1.4 KH₂PO₄) and placed in a 1 cm quartz cuvette inside a stirring thermojacketed sample holder of the fluorescence instrument and equilibrated at 25° C. for 15 minutes with gentle stirring. To the cuvette, through an injector port, 25 ml of the peptide dissolved in DMSO was added with continuous fluorescence monitoring at 520 nm (excitation at 470 nm). The observed data were then normalized for comparison into the fractional change in fluorescence ((I₀-Iₜ)/(I₀-I_{∞})) (V. E. Carmichael et al, in J.Am. Chem. Soc. (1989), vol. 111, pages 767-769)).

Control studies, monitoring the release of carboxyfluorescein dye entrapped in liposomes, indicated that the collapse of the pH gradient was not due to the rupturing of the liposomes nor due to the small amounts of organic solvents (<2% DMSO) employed in these studies. Furthermore, the control peptide cyclo[-(Gln-D-Leu)₄] that lacks the appropriate surface characteristics for partitioning into the liposomes, does not display any ion transport activity under similar conditions. A second control peptide cyclo[-(^{Me}N-D-Ala-Phe)₄-] that has the desirable hydrophobic surface characteristics but lacks the propensity for participating in extended hydrogen bonding network, was also designed and tested for ion transport activity. The ring structure of this peptide is N-methylated on one face. Such N-methylation does not adversely affect the ability of the peptide to interact with liposomal membranes but predisposes peptides toward a dimeric cylindrical structure that cannot span a normal liposomal membrane. Thus, although the peptide has been shown to partition effectively into liposomes, it does not promote proton transport activity in the above vesicle experiments. Together, these experiments support the idea that not only are the side chains displayed on cyclic peptides important for membrane interaction, but also the peptide backbone should be able to participate in extended intermolecular hydrogen bonding, for example, to facilitate membrane permeabilization.

Methods of Use

The present invention is directed to methods of treating or preventing microbial infections in a mammal, as well as other animals, such as farm animals and birds. These methods include administering to the animal a therapeutically effective amount of a cyclic peptide of the present invention. Treatment of, or treating, microbial infections is intended to include the alleviation of or diminishment of at least one symptom typically associated with the infection. The treatment also includes alleviation or diminishment of more than one symptom. Ideally, the treatment cures, e.g., substantially kills the microbes and/or eliminates the symptoms associated with the infection.

Microbial infections that can be treated by the present cyclic peptides include infections by any target microbial organisms that can infect a mammal or other animal. Such target microbial organisms include essentially any single cell organism or parasite that has a cellular membrane and that can infect an animal, including mammals. For example, target microbial organisms include bacteria, fungi, yeast strains and other single cell organisms. Cyclic peptides are active against both gram-negative and gram-positive bacteria.

Hence, for example, infections of the following target microbial organisms can be treated by the present cyclic peptides: Aeromonas spp., Bacillus spp., Bacteroides spp., Campylobacter spp., Clostridium spp., Enterobacter spp., Enterococcus spp., Escherichia spp., Gastrospirillum sp., Helicobacter spp., Klebsiella spp., Salmonella spp., Shigella spp., Staphylococcus spp., Pseudomonas spp., Vibrio spp., Yersinia spp., and the like. Infections that can be treated by the present peptides include those associated with staph infections (Staphylococcus aureus), typhus (Salmonella typhi), food poisoning (Escherichia coli, such as O157:H7), bascillary dysentery (Shigella dysenteria), pneumonia (Psuedomonas aerugenosa and/or Pseudomonas cepacia), cholera (Vivrio cholerae), ulcers (Helicobacter pylori) and others. E. coli serotype 0157:H7 has been implicated in the pathogenesis of diarrhea, hemorrhagic colitis, hemolytic uremic syndrome (ITUS) and thrombotic thrombocytopenic purpura (TTP). The peptides of the invention are also active against drug-resistant and multiply-drug resistant strains of bacteria, for example, multiply-resistant strains of Staphylococcus aureus and vancomycin-resistant strains of Enterococcus faecium and Enterococcus faecalis.

Anti-microbial activity can be evaluated against these varieties of microbes using methods available to one of skill in the art. Anti-microbial activity, for example, is determined by identifying the minimum inhibitory concentration (MIC) of a cyclic peptide of the present invention that prevents growth of a particular microbial species. In one embodiment, anti-microbial activity is the amount of the peptide that kills 50% of the microbes when measured using standard dose or dose response methods.

The present invention also provides a method of evaluating therapeutically effective dosages for treating a microbial infection with cyclic peptides described and claimed herein that includes determining the minimum inhibitory concentration of a cyclic peptide at which substantially no microbes grow in vitro. Such a method permits calculation of the approximate amount of cyclic peptide needed per volume to inhibit microbial growth or to kill 50% of the microbes. Such amounts can be determined, for example, by standard microdilution methods. For example, a series of microbial culture tubes containing the same volume of medium and the substantially the same amount of microbes are prepared, and an aliquot of cyclic peptide is added. The aliquot contains differing amounts of cyclic peptide in the same volume of solution. The microbes are cultured for a period of time corresponding to one to ten generations and the number of microbes in the culture medium is determined. The optical density of the cultural medium can be used to estimate whether microbial growth has occurred—if no significant increase in optical density has occurred, then no significant microbial growth has occurred. However, if the optical density increases, then microbial growth has occurred. To determine how many microbial cells remain alive after exposure to the cyclic peptide, a small aliquot of the culture medium can be removed at the time when the cyclic peptide is added (time zero) and then at regular intervals thereafter. The aliquot of culture medium is spread onto a microbial culture plate, the plate is incubated under conditions conducive to microbial growth and, when colonies appear, the number of those colonies is counted.

According to the present invention, the cyclic peptides provided herein do not cause substantial or undesired toxicity against mammalian cells or the cells of otheranimals to be treated. Mammalian or bird red blood cell hemolysis is one way to measure whether a cyclic peptide can cause undesired toxicity against mammalian cells or the cells of other animals to be treated. If a cyclic peptide can self-assemble by association with a mammalian or animal cell membrane, the membrane may be disrupted. Red blood cells are conveniently used to test for membrane disruption, because they undergo hemolysis, which can be detected as the release of hemoglobin from the cell. Hemolysis assays can be performed by methods available to one of skill in the art. For example, after exposure to test compounds, the release of hemoglobin can be observed spectrophotometrically by observing the absorbance of light at wavelengths characteristic of hemoglobin, for example, at 543 nm. Control samples can be used, for example, the medium in which the cells are tested or maintained can serve as a zero blank. A second control can be used to determine the absorbance value for 100% lysis or hemolysis that can be a sample that is identical to the test mammalian cell sample but which had been sonicated to completely disrupt the cells. Additionally hemolytic agents such as mellitin or a variety of detergents can also be used to establidh 100% hemolysis of test red blood cells.

Screening Assays

Screening or other assays may be used to identify, confirm or evaluate cyclic peptides that can selectively interact with, rupture of kill a microbe or other cell type of interest. A wide variety of assays may be used for this purpose. In general, such an assay can involve contacting a microbe or other cell type of interest with at least one cyclic peptide and observing whether the cyclic peptide interacts with a microbe or other cell type of interest and/or has deleterious effects upon that microbe or other cell type.

Methods available in the art can be used for determining whether the cyclic peptides of the invention, for example, interact with the membrane of a cell type of interest. For example, cyclic peptides can be labeled with a reporter molecule that permits detection of the peptide. After labeling, the cyclic peptides can be contacted with the cell type of interest for a time and under conditions that permit binding or association of the peptide to cellular membranes. The cells can be washed with physiological solutions to remove unbound or unassociated cyclic peptides, and the microbes or cells can then be observed to ascertain whether the reporter molecule is bound or associated with the microbes, cells or cellular membranes. In another embodiment, one of skill in the art can test whether the cyclic peptide(s) can selectively penetrate the membranes of particular microbes or other selected cell types. This may be done, for example, by examining whether the reporter molecule remains associated with the cellular membranes of the microbe or cell type of interest or whether the reporter molecule becomes associated with the interior of the microbe or cell type of interest.

Reporter molecules that can be employed include any detectable compound or molecule available to one of skill in the art that is conjugated directly or indirectly to a cyclic peptide of the invention. The label may itself be detectable (e.g., radioisotope labels or fluorescent labels) or, in the case of an enzymatic label, may catalyze chemical alteration of a substrate compound or composition that is detectable or molecular epitopes for protein/antibody capture and detection.

Deleterious effects upon the microbe or cell type of interest can also be detected as an indication of an interaction between a cyclic peptide of the invention and the microbe or cell type of interest. Such deleterious effects may be confirmed by any evidence that the cyclic peptide has had an adverse or cytotoxic effect upon the microbe or cell type of interest. For example, one of skill in the art can test whether the cyclic peptide(s) kill the cell type, or cause membrane depolarization or permeabilization of the membranes of the microbes or cell types of interest.

Of particular interest are screening assays for cyclic peptides that have low toxicity for normal human or other animal cells but that have good anti-microbial properties (depolarizing or permeabilizing microbial cell membranes, lysing or otherwise killing microbes). Such cyclic peptides may or may not interact with normal human or other animal cells so long as they have few toxic effects on such normal human or other animal cells.

Generally, in the testing of multiple peptides a plurality of assays are performed in parallel with different cyclic peptides, which may be introduced at different concentrations to obtain a differential response to the various concentrations. Typically, at least one control assay is included in the testing. Such a control can be a negative control involving exposure of the microbes or cells of interest to a physiologic solution containing no cyclic peptide. Another control can involve exposure of the microbe or cell type of interest to a cyclic peptide that has already been observed to adversely affect the microbe or cell type of interest, or a second cell type that is related to the microbe or cell type of interest. Another control can involve exposing a microbe or cell type of interest to a known therapeutic agent that has a desired affect on the microbe or cell type of interest, for example, an anti-microbial or cytotoxic agent with known efficacy at a particular concentration or dosage. One of skill in the art can readily select control compounds and conditions that facilitate such evaluations.

Candidate cyclic peptides are obtained from a wide variety of sources including libraries of cyclic peptides generated as described herein. Cyclic peptides can also be individually or rationally designed and synthesized to have specific structural features selected by one of skill in the art.

Any cell type available to one of skill in the art can be screened by these methods. For example, any microbial or mammalian or animal cell type can be screened to assess whether the cyclic peptides of the invention can selectively or non-selectively interact therewith. Such microbial cell types include any single cell organism that is capable of autonomous replication. Examples include any bacterial, fungal and yeast cell types. Mammalian or other animal cell types can also be screened to ascertain whether the peptides of the invention interact therewith and/or to determine or confirm whether the peptides of the invention do not interact, bind, lyse, kill or otherwise adversely affect the viability of the mammalian or other animal cell type of interest.

In one embodiment, mammalian red blood cells are screened with the cyclic peptides to ascertain whether the cyclic peptides have an adverse effect on the red blood cells. The membranes of red blood cells tend to be more sensitive to lysis than many other mammalian cell types. Hence, red blood cells are a useful cell type for quickly screening whether a cyclic peptide would be expected to have any adverse effects on these or other mammalian cell types. Methods of screening for mammalian cell lysis are available in the art. For example, using procedures described herein, red blood cells can be tested to ascertain whether hemolysis has occurred upon exposure to at least one cyclic peptide of the invention. When it is established that a cyclic peptide causes little or no undesired hemolysis of red blood cells, it may be tested against other mammalian cell types or used for in vivo testing in standard animal models.

Conditions for screening cyclic peptides include conditions that are used by one of skill in the art to grow, maintain or otherwise culture cell types of interest. Cell types of interest should be assayed under conditions where they would be healthy but for the presence of the cyclic peptide(s). Controls can be performed where the cell types are maintained under the selected culture conditions and not exposed to a cyclic peptide, to assess whether the culture conditions influenced the viability of the cells. One of skill in the art can also perform the assay on cells that have been washed in simple physiological solutions, such as buffered saline, to eliminate, or test for, any interaction between the cyclic peptides or cells and the components in the culture media. However, culture conditions for the assays generally include providing the cells with the appropriate concentration of nutrients, physiological salts, buffers and other components typically used to culture or maintain cells of the selected type. A variety of other reagents may be included in the screening assay. These include reagents like salts, neutral proteins, albumin, serum (e.g. fetal calf serum) that are used to mimic the physiologic state of the cell types of interest. Conditions and media for culturing, growing and maintaining mammalian cells and bacterial or other microbial cells are available to one of skill in the art.

The selected reagents and components are added to the assay in the order selected by one of skill in the art. In general, the cyclic peptides are added last to start the assay. Assays are performed at any suitable temperature, typically between 4° C. and 40° C. Temperatures generally range from about room temperature (about 20° C.) to about 37° C. Incubation periods are selected to ascertain the optimal range of activity or to insure that the cyclic peptides do not adversely affect unwanted cell types. However, incubation times can be optimized to facilitate rapid high-throughput screening. Typically incubation times are between about 1 minute and about 24 hours, other times range from about 5 minutes to about 8 hours.

Cyclic peptides having the desired selectivity and activity during in vitro screening or evaluation may be tested for activity and/or lack of toxicity in vivo in an appropriate animal model. Such animal models include mice, rats, rabbits, cats, dogs, pigs, goats, cattle or horses. For example, the mouse and the rat are convenient animal models for testing whether cyclic peptides of the invention have toxic effects and/or to determine whether the cyclic peptides can combat a microbial infection.

One of skill in the art can readily perform in vivo screening of the cyclic peptides of the invention. For toxicity testing, a series of cyclic peptides at different test dosages can be separately administered to different animals. A single dose or a series of dosages can be administered to the animal. A test period is selected that permits assessment of the effects of the peptide(s) on the animal. Such a test period may run from about one day to about several weeks or months.

The effect of a cyclic peptide(s) on an animal can be determined by observing whether the peptide adversely affects the behavior (e.g., lethargy, hyperactivity) and physiological state of the animal over the course of test period. The physiological state of the animal can be assessed by standard procedures. For example, during the test period one of skill in the art can draw blood and collect other bodily fluids to test, for example, for various enzymes, proteins, metabolites, and the like. One of skill in the art can also observe whether the animal has bloating, loss of appetite, diarrhea, vomiting, blood in the urine, loss of consciousness, and a variety of other physiological problems. After the test period, the animal can be sacrificed and anatomical, pathological, histological and other studies can be performed on the tissues or organs of the animal.

In general, to determine whether one or more cyclic peptides of the invention can combat a microbial infection, mice or other test animals are infected with the selected microbe and a selected test dosage of one or more cyclic peptides is administered thereafter at predetermined elapsed time periods or intervals. Test animals are observed over the course of several days to weeks to ascertain whether the cyclic peptide protects the animals from the microbial infection. At the end of the test period, the test animals can be sacrificed and examined to ascertain whether the cyclic peptide has optimally protected the test animals from infection and/or to determine whether any adverse side effects have occurred.

Prior to administration, the microbe can be washed in simple physiological solutions to remove toxins or other components that may adversely affect the mice. For example, microbial cells can be grown at 37° C. with agitation for 12 hours to a stationary phase. Microbial cells can be collected by centrifugation, washed twice with saline or phosphate buffered saline (PBS) and resuspended or diluted to a convenient cell density, for example, a cell density of about of about 3-5×10⁷ cfu/ml. Several animals to be tested with varying amounts of each peptide five are infected with a small volume of the microbe (e.g. about 0.1 to about 1 ml). Infection can be performed orally, intraperitoneally, intravenously or by some other route selected by one of skill in the art. After infection, the animals are allowed to rest for a short time and then each animal is treated with a different dose of peptide. The animals are monitored for several days to weeks.

Controls are used to establish the effects of the microbe when the cyclic peptide is not administered. Other controls can also be performed, for example, the safety and efficacy of the present cyclic peptides can be compared to that of known anti-microbial agents (e.g., penicillin, kanamycin, vancomycin, erythromycin, etc.).

The invention further provides a method of identifying or evaluating a D-, L-α-cyclic peptide or a β-peptide capable of selective association with a target biomolecule. Such target biomolecules can include, for example, intracellular, extracellular or membrane-associated proteins, enzymes, nucleic acids, receptors, organelles and the like. This method can involve contacting a solution of cyclic peptides with the target biomolecule under hydrogen bond-promoting conditions and determining whether the peptides selectively associate with the desired biomolecules and possess biological activity of interest.

The target biomolecule can be displayed, for example, on the surface of a living cell, on the surface of a genetically engineered cell or on the surface of a liposome. Alternatively, the peptide can be contacted with the target biomolecule under other desired assay conditions available to one of skill in the art.

Cyclic peptides having good anti-microbial properties in vitro and/or in vivo that also have substantially no undesired toxicity against unwanted cell types are particularly good candidates for the prepartion of appropriate dosage forms, as described in more detail below.

Dosages, Formulations and Routes of Administration for the Peptides

The peptides of the invention, including their salts, are administered so as to achieve a reduction in at least one symptom associated with an infection, indication or disease, or a decrease in the amount of antibody associated with the indication or disease.

To achieve the desired effect(s), the peptide, a variant thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the cyclic peptide chosen, the disease, the weight, the physical condition, the health, the age of the mammal, whether prevention or treatment is to be achieved, and if the peptide is chemically modified. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the peptides of the invention may be essentially continuous over a preselected period of time or may be in a series of spaced doses. Both local and systemic administration is contemplated.

To prepare the composition, peptides are synthesized or otherwise obtained, purified as necessary or desired and then lyophilized and stabilized. The peptide can then be adjusted to the appropriate concentration, and optionally combined with other agents. The absolute weight of a given peptide included in a unit dose can vary widely. For example, about 0.01 to about 2 g, or about 0.1 to about 500 mg, of at least one peptide of the invention, or a plurality of peptides specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the cyclic peptides of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic peptides of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic peptides may also be formulated for sustained release (for example, using microencapsulation, see WO 94/ 07529, and U.S. Pat. No.4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic peptides of the invention are prepared for oral administration, they are generally combined with a pharmaceutically acceptable carrier, diluent or excipient to form a pharmaceutical formulation, or unit dosage form. For oral administration, the peptides may be present as a powder, a granular formulation, a solution, a suspension, an emulsion or in a natural or synthetic polymer or resin for ingestion of the active ingredients from a chewing gum. The active peptides may also be presented as a bolus, electuary or paste. Orally administered therapeutic peptides of the invention can also be formulated for sustained release, e.g., the peptides can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic peptides of the invention can be prepared by procedures known in the art using well-known and readily available ingredients. For example, the peptide can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the cyclic peptides of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one cyclic peptide of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more peptides of the invention are designed to resist disintegration in the stomach and dissolve in the more neutral to alkaline environment of the duodenum.

The therapeutic peptides of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic peptides of the invention can also take the form of an aqueous or anhydrous solution or dispersion, or alternatively the form of an emulsion or suspension or salve.

Thus, the therapeutic peptides may be formulated for parenteral administration (e.g., by injection, for example, bolus injection or continuous infusion) and may be presented in unit dose form in ampules, pre-filled syringes, small volume infusion containers or in multi-dose containers. As noted above, preservatives can be added to help maintain the shelve life of the dosage form. The active peptides and other ingredients may form suspensions, solutions, or emulsions in oily or aqueous vehicles, and may contain formulatory agents such as suspending, stabilizing and/or dispersing agents. Alternatively, the active peptides and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Also contemplated are combination products that include one or more cyclic peptides of the present invention and one or more other anti-microbial agents. For example, a variety of antibiotics can be included in the pharmaceutical compositions of the invention, such as aminoglycosides (e.g., streptomycin, gentamicin, sisomicin, tobramycin and amicacin), ansamycins (e.g. rifamycin), antimycotics (e.g. polyenes and benzofuran derivatives), β-lactams (e.g. penicillins and cephalosporins), chloramphenical (including thiamphenol and azidamphenicol), linosamides (lincomycin, clindamycin), macrolides (erythromycin, oleandomycin; spiramycin), polymyxins, bacitracins, tyrothycin, capreomycin, vancomycin, tetracyclines (including oxytetracycline, minocycline, doxycycline), phosphomycin and fusidic acid.

Additionally, the peptides are well suited to formulation as sustained release dosage forms and the like. The formulations can be so constituted that they release the active peptide, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic peptides of the invention can be delivered via patches or bandages for dermal administration. Alternatively, the peptide can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic peptides in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic peptide may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The peptides of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific infection, indication or disease. Any statistically significant attenuation of one or more symptoms of an infection, indication or disease that has been treated pursuant to the method of the present invention is considered to be a treatment of such infection, indication or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic peptides of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the peptides of the present invention specific for the indication or disease to be treated. Dry aerosol in the form of finely divided solid peptide or nucleic acid particles that are not dissolved or suspended in a liquid are also useful in the practice of the present invention. Peptides of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic peptides of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmnoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, antihistamines, bronchodilators and the like, whether for the conditions described or some other condition.

The present invention further pertains to a packaged pharmaceutical composition for controlling microbial infections such as a kit or other container. The kit or container holds a therapeutically effective amount of a pharmaceutical composition for controlling microbial infections and instructions for using the pharmaceutical composition for control of the microbial infection. The pharmaceutical composition includes at least one cyclic peptide of the present invention, in a therapeutically effective amount such that microbial infection is controlled.

The invention is further illustrated by the following non-limiting Examples.

### EXAMPLE 1

### Materials and Methods

Solid Phase Peptide Synthesis

Solvents and reagents: Acetonitrile (ACN, optima grade), dichloromethane (DCM, ACS grade), N,N-dimethylformamide (DMF, sequencing grade), diethylether (Et₂O, ACS grade), N,N-diisopropylethylamine (DIEA, peptide synthesis grade) were purchased from Fisher and used without further purification. Trifluoroacetic acid (TFA, New Jersey Halocarbon), 2-(1H-benzotriazol-1-yl)- 1,1,3,3-tetramethyluronium hexafluorophosphate (HBTU, Richelieu Biotechnologies), benzotriazole-1-yl-oxy-tris-pyrrolidino-phosphonium hexafluorophosphate (PyBOP, Novabiochem) were used as obtained. Commercially available amino acids and resins were used as obtained from Bachem, Novabiochem or Advanced Chemtech. The side-chain protections were as follows. For Fmoc synthesis: Arg (Pbf), His (Boc), Lys (Boc), Ser (t-Bu) and Thr (t-Bu). All other chemicals were used as obtained from Aldrich, Acros, Sigma or Fluka.

Peptide synthesis and cyclization: Linear protected peptides were synthesized on polystyrene solid support using a trytil-functionalized resin and cyclized in the solution phase. The first Fmoc-protected amino acid was loaded onto a Cl-trytil-resin via its α-carboxylate group following standard procedures. Briefly, dry Fmoc-amino acid (1.2 eq. with respect to resin loading) was dissolved in DCM (dried over NaHCO₃, 20 mL/g of resin) and DIEA (4 eq.). This mixture was added to a fresh commercially available Cl-trytil resin (loading 0.6-1.2 mmol/g) and continuously shaken for 2 hour, followed by sequential washings with DCM (3×20 mL), MeOH/DIEA/DCM (1:2:17, 3×15 mL) and DCM (3×20 mL). Loading of the first amino acid was estimated by removing the Fmoc group with base from a measured amount of resin and then measuring the UV absorption and concentraion of the product in a known sample volume. The rest of the amino acids were sequentially introduced following standard Fmoc protocols. J. M. a. Y. Stewart, J. D., Solid Phase Peptide Synthesis 1984. After the last coupling, N-terminal Fmoc group was removed by treatment with 20% Piperidine/DMF. The peptide containing resin was washed with DMF (3×20 mL), then with DCM (3×20 mL). The side chain protected linear peptide was cleaved from the resin by sequential shaking with several portions (10-20) of TFA/DCM mixture (1/99) collecting the solution into a flask, containing pyridine (2 mL) and MeOH (5 mL). The completion of the cleavage is achieved when the resin changed its color to dark red. Then the resin was washed with DCM (2×10 mL) and MeOH (2×10 mL). These washings were combined with collected TFA cleavage solutions. The solvent was evaporated under vacuum (1 mm Hg) and the purity of the linear peptide was assessed by MALDI-MS and HPLC. In most cases, purity of the crude linear peptide allowed cyclization without prior purification. Otherwise, linear peptides were purified by reverse phase high performance liquid chromatography (RP-HPLC/C4 or C18, 0.1% TFA in ACN/H₂O).

Cyclization was performed in DMF at a peptide concentration of 1-5 mM using a mixture of PyBOP (5 eq. with respect to crude peptide) and DIEA (40 eq.). The amount of DIEA was adjusted to achieve an apparent pH 9-10, which was assessed by applying a drop of reaction mixture to a wet pH paper. Reaction was followed by MALDI-MS and HPLC and in most cases it was complete in less than 2 h. Then DMF was removed by evaporation under vacuum (1 mm Hg) at temperatures less that 30° C. and the residue was dried under vacuum (0.1 mm Hg) overnight.

For deprotection of the cyclic peptide side chains, the dried crude peptide was dissolved in a mixture of TFA/PhOH/H₂O/thioanisole/EDT/TIS (81.5:5:5:2.5:1) (˜100 mL/g of peptide) at rt for 1-3 hours. The completion of the reaction was followed by HPLC and MALDI-MS. The TFA solution was concentrated by 5 times by evaporation under vacuum (1 mm Hg), from which the peptide was precipitated by adding it to an ice-cold Et₂O. The purity of dried crude peptides was assessed by HPLC and MALDI-MS. The crude peptides can be partially purified by dissolving in boiling ACN/water/HCl mixture (30/70/0.1) and cooling the turbid solution in a fridge. In case of peptide high solubility in this mixture, the precipitate can be obtained by adding acetone (3 vol. eq.) to the above solution. Further purification was achieved by preparative reverse phase HPLC (C4, radial compression column, Waters) using, for example, a gradient of eluent A (0.1% HCl in 99% H₂O/1% ACN (v/v)) and eluent B (0.07% HCl in 90% ACN/10% H₂O (v/v)) using a flow rate of 24 m/min.

Combinatorial Peptide Synthesis: Mixture Libraries

Materials for peptide synthesis by Boc chemistry were purchased from a variety of commercial sources. For the synthesis of the first generation libraries of peptides, N-Boc-α-Fmoc-glutamic acid was loaded onto methyl-benzhydrylamine (MBHA) resin through its side chain carboxylate, then the resin was split into four equimolar fractions of 0.25 mmol each for the rest of the synthesis. To each fraction was coupled one of four N-Boc-protected D-amino acids, lysine, arginine, glutamic acid, or serine (4 equivalents), using (O-(7-azabenzotriazol-1-yl)- 1,1,3,3-) tetramethyluronimum hexafluorophosphate (HATU, 4 equivalents), 1-hydroxy-7-azabenzotriazole (HOAT, 4 equivalents), and diisopropylethylamine (DIEA, 6 equivalents) in DMF. To positions 3 through 7 were coupled a mixture of N-Boc-protected alanine, leucine, valine, phenylalanine, and tryptophan, at a molar ratio of 1.35:1.97:4.47:1:1 (10 equivalents versus resin loading) to compensate for the difference in the coupling efficiencies (Pinilla, C.; Appel, J. R.; Blondelle, S. E.; Dooley, C. T.; Eichler, J.; Ostresh, J. M.; Houghten, R. A. “Versatility of positional scanning synthetic combinatorial libraries for the identification of individual compounds.” Drug Dev. Res. 1994, 33, 133-145).

Standard procedures were utilized for peptide synthesis except that the odd positions contained amino acids of L-chirality and the even positions amino acids with D-chirality. In position 8, a mixture of N-Boc-protected D-amino acids, lysine, arginine, glutamic acid, and serine, at a molar ratio of 2.24:2.34:1.31:1 was coupled (10 equivalents versus resin loading). After removal of the N-terminal Boc from the peptide chain with neat TFA, the α-Fmoc on the carboxyl terminus of the glutamic acid was removed with 30% piperidine in DMF to allow cyclization of the peptide on the resin, using HATU (2 equivalents), HOAT (2 equivalents), and DIEA (4 equivalents) in DMF/DMSO and/or benzotriazole-1-yl-oxy-trix-pyrrolidino-phosphonium hexafluorophosphate (PyBOP, 1 equivalent), N-hydroxybenzotriazole, (HOBT, 1 equivalent), and DIEA (2 equivalents) in dry 2M LiBr/tetrahydrofuran over a period of 24-48 hours. Peptides were cleaved by standard high HF cleavage procedure, washed with ether then extracted with 10% acetic acid followed by DMF. The extracts were pooled and lyophilized.

The specified amino acids of the peptide library sequences showing the greatest biological activity (lowest MIC value) were retained for the generation of the next set of libraries. Subsequent generations of peptide libraries were synthesized in a similar fashion, with the splitting of the resin after the coupling of the specified amino acids determined from the previous generation. The peptides of the combinatorial libraries were identified by electrospray-mass spectrometry (ES-MS) or MALDI-TOF mass spectrometry.

When a peptide library, peptide pool or crude preparation of a peptide showed activity, individual peptides were re-synthesized, HPLC-purified and tested again for activity. For example, a crude preparation of a peptide having a low minimum inhibitory concentration value, cyclo[D-Arg-L-Gln-D-Arg-L-Trp-D-Trp-L-Leu-Trp-L-Trp] (SEQ ID NO:10), was resynthesized, HPLC-purified, and tested for anti-microbial activity, and was found to have biological activity similar to the crude.

Single-Compound per Bead Combinatorial Cyclic Peptide Libraries

Cyclic D, L-α-peptide combinatorial libraries were prepared using a one-bead-one-compound strategy on macrobeads by a split and pool method. See, K. S. Lam, M. Lebl, V. Krchnak, “The ‘one-bead-one-compound’ combinatorial library method,” Chem. Rev. 1997, 97, 411-448. Each bead contained a single sequence and was dispersed into microtitre plates using a density of one bead per well. Cleavage of the peptide from a single bead provided about 70-80 μg of peptide per well. This amount of peptide could be used for approximately 100 in vitro anti-microbial assays. Mass spectrometric peptide sequencing strategies were used for rapid identification of selected peptide species within a given library.

Solid-phase peptide synthesis was performed on polystyrene macrobeads functionalized with a TFA-labile trityl linker, which considerably facilitated the synthesis, handling, solid-phase cyclization, and final side chain deprotection and peptide isolation. The growing peptide chain was linked through the first amino acid side chain (for example lysine or histidine) to the trityl moiety allowing for selective “head-to-tail” cyclization of the completed peptide sequence on solid support. The α-carboxyl group of the first N-α-Fmoc amino acid was protected as an allyl ester. Resin loading and peptide chain elongation was performed under standard Fmoc solid phase peptide synthesis conditions using chlorotrityl polystyrene macrobead resin (500-560 um, Peptides International) as the solid support, with HBTU as a coupling reagent and 20% piperidine in DMF for Fmoc deprotection. After completion of the final amino acid coupling, the resin was exposed to palladium tetrakis(triphenylphosphine) and N-methyl morpholine to remove the C-terminal allyl protecting group. Subsequent N-terminal Fmoc deprotection followed directly by cyclization with PyBopg, provided the desired cyclic peptide generally in high yields. Single macrobeads was dispensed into discrete wells in microtitre plates manually or by using a bead dispenser. The protected cyclic peptide was released from the solid support and deprotected in one step using a 95% TFA (5% cation scavengers) solution. After cleavage, the solvent was removed in vacuo to yield cyclic peptide with high purity. Cleavage conditions and work-up procedures were generally optimized to eliminate non-volatile scavengers and potentially deleterious side products from the final peptide. Peptide libraries obtained by the above procedure were sufficiently pure for use in anti-microbial selection assays.

Materials: Acetonitrile (HPLC grade), dichloromethane (optima grade), dicyclohexylamine (DCHA), diethyl ether (anhydrous), dimethylformamide (sequencing grade), diisopropylethylamine (DIPEA, peptide synthesis grade), and piperidine (anhydrous) were purchased from Fisher and used without further purification. Trifluoroacetic acid (TFA, New Jersey Halocarbon), and 2-(1-H-benzotriazol-1-yl)-1,1-,3,3-tetramethyluronium hexafluorophosphate (HBTU, Novabiochem), benzotriazole 1-yl-oxy-tris-pyrrolidino-phosphonium hexafluorophosphate (PyBOP, Novabiochem) were used without further purification. Tetrakistriphenylphosphine palladium(0) was purchased from Strem Chemicals. Commercially available N-Fmoc amino acids for solid-phase peptide synthesis and trityl chloride PS (1% DVB, substitution 0.5-1.05 mmol g⁻¹) resin were used as obtained from Novabiochem or Bachem. Trityl chloride macrobead resin was obtained from Peptides International.

Prpeapartion of Fmoc-Lysine(Boc)-Oallyl. Fmoc-Lysine(Boc)-OAllyl was made according to the protocol of Kates, S. A.; Sole, N. A.; Johnson, C. R.; Hudson, D.; Barany, G.; Albericio, F. Tetrahedron Lett. 1993, 34, 1549-1552. Fmoc-Lys-(Boc)-OH (5 g, 10.6 mmol) was added to allyl bromide (25 mL, 0.29 mol), followed by DIPEA (3.73 mL). This mixture was heated at 90° C. for 1 h. The reaction was allowed to cool, concentrated by rotary evaporation, and after dilution with ethyl acetate was washed with 2×0.1 N HCl, 2× saturated sodium bicarbonate at pH<9.5, followed by brine. The organic layer was filtered through a pad of silica gel and concentrated to afford a solid. This solid was washed with ether to provide a white powder that was used directly in the next step.

Deprotection of Side Chain Boc Protecting Group. An appropriate amount of Fmoc-Lys(Boc)-OAllyl for a resin loading of 0.5 mmol g⁻¹ was placed in a round bottom flask. Sufficient dichloromethane to dissolve the solid was added followed by an equivalent amount of TFA. After stirring for 1 h the solution was evaporated and the residue of Fmoc-Lys-OAllyl was dried in vacuo.

Resin loading. Trityl chloride resin was swollen in dry deacidified (Na₂CO₃) dichloromethane for 20 min. A solution of Fmoc-Lys-OAllyl in dichloromethane was added to the resin, immediately followed by 4 eq. of DIPEA. After shaking for 2 hours the resin was washed resin with dichloromethane, then shaken with 10% MeOH: 10% DIPEA: 80% dichloromethane for 10 min. After washing with dichloromethane and drying in vacuo the resin loading was evaluated based on Fmoc released monitored by UV absorption at 290 nm.

Peptide Synthesis. Peptides were synthesized using standard solid-phase Fmoc protocols (see Wellings, D. A.; Atherton, E. Methods Enzymol. 1997, 289, 4467) on the Fmoc-Lys-OAllyl loaded trityl resin. Following synthesis of the linear peptide the resin was swollen in dry dichloromethane for 20 min. To the resin was added a degassed solution of 0.5 eq. Pd(PPh₃)₄ in 90% CHCl₃: 10% 4-methylmorpholine. After shaking under Ar for 5 hours the resin was washed with a solution of 1% sodium dimethylthiocarbamic acid in DMF (3×2 min), 1% DIPEA (3×2 min) in DMF. After the final Fmoc deprotection (25% piperidine in DMF, 2×10 min), the resin was washed thoroughly with DMF (3×3 min), 10% DIPEA/DMF (3×3 min), 0.8 M LiCl/DMF (3×3 min). The resin was treated with 5 eq. PyBOP, 5 eq. HOAt, 20 eq. DIPEA in 0.8 M LiCl/DMF for at least 12 hours. After washing with DMF (3×3 min), DCM (2×3 min) followed by MeOH the peptide was cleaved from the resin and deprotected with 2.5% TIS: 2.5% H₂O: 95% TFA. Peptides were recovered by precipitation with ether or by evaporation of the cleavage mixture.

Antimicrobial Assay

The antimicrobial activity of the peptides was determined using a broth dilution assay essentially as descried in the guidelines of the National Committee for the Control of Laboratory Standards (NCCLS) [National Committee for Clinical Laboratory Standards. Methods for dilution antimicrobial susceptibility tests for bacteria that grow aerobically. Fourth edition. Approved Standard (1997). Document M7-A4. (NCCLS, Villanova, Pa., 1997]. Test tubes (macrodilution method) or microtiter plates (microdilution method) containing two-fold serial dilution of peptides were inoculated with various bacterial cultures. Controls included non-inoculated medium (sterility), vehicle control, and various commercially available antibiotics for which minimal inhibitory concentrations against tested organisms were known. In vitro results using both methods were reproducible and in all cases MICs from the macrodilution assays were equal to, or one dilution below, the inhibitory concentrations determined by microdilution tests (See:V. Lorian, Antibiotics in Laboratory Medicine, Williams and Wilkins, Baltimore 1991). The microdilution method has the advantages of requiring less amount of peptide for each assay and the possibility of multiple simultaneous inoculations and was used in most studies. Several of the strains of bacteria tested are further described in Table 3.

TABLE 3

| Species | Strain | Type | Special Properties |
|---|---|---|---|
| Bacillus cereus | ATCC 11778 | Gram | |
| | | positive | |
| Enterococcus | ATCC 51299 | Gram | Vancomycin resistant |
| faecalis | | positive | (vanB); resistant to |
| | | | vancomycin, gentamicin |
| | | | and streptomycin. |
| Enterococcus | SP180 | Gram | Vancomycin resistant |
| faecium | | positive | (vanA); resistant to multiple |
| | | | antibiotics |
| Staphylococcus | OS2 | Gram | |
| aureus | | positive | |
| Staphylococcus. | ATCC 33591 | Gram | Methicillin-resistant. |
| aureus | | positive | |
| Staphylococcus. | ATCC 33592 | Gram | Methicillin-resistant; |
| aureus | | positive | resistant to multiple |
| | | | antibiotics |
| Streptococcus | ATCC 6301 | Gram | |
| pneumoniae | | positive | |
| Listeria | ATCC 19115 | Gram | |
| monocytogenes | | positive | |
| Escherichia | K12 | Gram | Reference strain |
| coli | | negative | |
| Escherichia | JM109 (DE3) | Gram | |
| coli | | negative | |
| Escherichia | EL744 K12 | Gram | Lacks key efflux pump |
| coli | (tolC::kan) | negative | component |
| Hemophilus. | ATCC 49247 | Gram | Reference strain |
| influenzae | | negative | |
| Salmonella | ATCC 14028 | Gram | |
| typhimurium | | negative | |

MIC Determination; Broth Dilution Method

Preparation of peptide solutions. Stock peptide solutions were prepared in 5% DMSO in aq. sucrose (9%). Determination of peptide concentrations was done by quantitative HPLC analysis using known concentrations of internal standards and/or by measuring UV absorption of tryptophane-containing peptide solutions in H₂O (λ=280 ε_{Trp}: 5690). Serial 2-fold dilutions were made in the above DMSO/aq. sucrose (9%) mixture with concentrations ranging approximately 400-2 μg/ml and aliquots were dispensed in test tubes (100 μl) for the macrodilution test or in microtiter plates (20 μl) for the microdilution assays.

Inoculum preparation. Overnight cultures of different microorganisms grown in suitable media were diluted 4000 times to an approximate inoculum size of 2.5×10⁵ cfu/ml.

Macrodilution method. Macrodilution methods were performed using procedures similar to those described in the National Committee for Clinical Laboratory Standards. Methods for dilution antimicrobial susceptibility tests for bacteria that grow aerobically. Fourth edition. Approved Standard (1997). Document M7-A4. (NCCLS,Villanova, Pa., 1997). Two ml aliquots of the above inoculum were dispensed to test tubes containing different peptide solutions. After incubation at 37° C. with shaking for 18 hours the lowest concentration at which no bacterial growth was observed was recorded as the MIC.

Microdilution method. Microdilution methods were performed using procedures similar to those described in the National Committee for Clinical Laboratory Standards. Methods for dilution antimicrobial susceptibility tests for bacteria that grow aerobically. Fourth edition. Approved Standard (1997). Document M7-A4. (NCCLS,Villanova, Pa., 1997. Eighty μl aliquots of the above inoculum were dispensed into 96-well microtiter plates containing different peptide solutions and incubated for 18 h at 37° C. with shaking (plates were sealed with parafilm to avoid excessive evaporation of culture medium) and MIC's were recorded. Each assay was performed at least twice and errors were typically ±one dilution.

MBC Determination

Minimum bactericidal concentrations (MBC) were determined according to the guidelines of the National Committee for the Control of Laboratory Standards (NCCLS). National Committee for Clinical Laboratory Standards. Methods for Determining Bactericidal Activity of Antimicrobial Agents. Approved Standard (1999). Document M26-A. (NCCLS, Villanova, Pa., 1999). Fifty μl aliquots from MIC, 2×MIC and 4×MIC assay wells were removed and plated in antibiotic-free agar plates by using the lawning technique. Growth and sterility controls were sampled in the same manner. The lawned plates were incubated for 24-48 hours and the MBC was determined as the lowest concentration at which 99.9% killing was achieved.

Membrane Depolarization Studies

Membrane depolarization was monitored by a change in fluorescence emission intensity of the cyanine membrane potential-sensitive dye 3,3′-dipropylthiadicarbocyanide iodide (diSC₃). Intact S. aureus were grown at 37° C. with agitation to mid log-phase (O.D.₆₀₀=0.5). Cells were centrifuged and washed once with buffer (20 mM glucose, 5 mM HEPES pH 7.3) and resuspended to an O.D.₆₀₀ of 0.05 in a similar buffer containing 0.1 M KCl. Cells were incubated with 1 μM diSC₃ until a stable reduction of fluorescence was achieved (approximately 15 min), indicating incorporation of the dye into the bacterial membrane. Peptides were added from stock solutions (1 mg/ml) dissolved in 5% DMSO in aqueous sucrose (9%) to achieve desired 0.1 to 10×MIC concentrations.

Hemolytic Sensitivity

Selectivity of cyclic peptides for bacterial over mammalian cells was evaluated by measuring red blood cell hemolytic activities as described in Tosteson, M. T. Holmes, S. J., Razin, M. and Tosteson, D. C. Melittin Lysis of Red Cells, J. Membrane Biol. 87, 35-44(1985). Heparinized murine blood was centrifuged at 1000×g for 10 minutes. The supernatant and the buffy coat were removed. Erythrocytes were washed three times with 0.9% saline solution and then resuspended to a concentration of 5% in saline containing 10% FBS (v/v). Red blood cells were then treated with serial dilutions of test peptides in a 96 well plate at 37° C. for 30 minutes. Control samples included a saline solution arid 1% Triton X-100 as 0 and 100% hemolysis, respectively. In some cases, mellitin was used as a further control. Mellitin is a linear peptide that is hemolytic against mammalian red blood cells in vitro at a concentration of about 10 μg/ml. Plates were centrifuged at 1000 g for 10 minutes. Aliquots of the supernatant were diluted 2 times with saline solution and the absorbance was measured at 560 nm.

Preparation of Peptide Solutions for In Vivo Experiments

Stock peptide solutions were prepared in aqeous sucrose (9%).. To facilitate dissolution of the peptide, the initial suspension was sonicated for 15-20 min. The obtained solution was sterilized by passage through a sterile 0.45 μm filter (COSTAR, μStar, Corning Inc.). Determination of peptide concentrations was done by UV absorption in H₂O (λ=280 ε_{Trp}: 5690 cm⁻¹M⁻¹ for tryptophan-containing peptides) of different aliquots and found to be 60-70% of the one corresponding to w/v. Alternatively, the concentrations of various peptide stock solutions were determined by quantitative HPLC analysis using known concentrations of internal standards. Further the peptide solutions were appropriately diluted with sterile aq. sucrose (9%).

Bacteria Preparation for In Vivo Protection Testing

Bacteria were prepared for in vivo using procedures similar to those as previously described. See V. Lorian, Antibiotics in Laboratory Medicine, Williams and Wilkins, Baltimore 1991. S. aureus MRSA bacteria (ATCC 33591) were grown at 37° C. in 5 mL of Antibiotic Medium-3 (AM-3, Difco Laboratories) with agitation for 12 hours to a stationary phase. Cells were collected by centrifugation, washed twice with saline solution and resuspended in about 10 mL of saline to an O.D.₆₅₀ of 1.2. This suspension was diluted ten times in a sterilized 5% mucin (Difco) in saline to a concentration of 2-4×10⁷ cfu/ml (actual inoculum size was verified by colony counts on agar plates).

Vancomycin resistant Enterococcus faecium (VREF) bacteria (ATCC 51575) were grown at 37° C. in 60 mL of Brain Heart Infusion medium (BHI, Difco Laboratories) with agitation for 16 hours to a stationary phase. Cells were collected by centrifugation, washed twice with saline solution and resuspended in 6 mL of saline to a theoretical O.D.₆₅₀ of 9-10 (determined by taking OD.₆₅₀ of 10-20 fold diluted samples and correlating it to the original concentration). This suspension was diluted 15 times in a sterilized 5% mucin (Difco) in saline to a concentration of 5-7×10⁸ cfu/ml (actual inoculum size was verified by colony counts on agar plates).

In Vivo Animal Studies

Peptides of the invention were tested in mice to ascertain whether they would protect the mice against bacterial infection using procedures similar to those described in N. Frimodt-Moller et al., The Mouse Peritonitis/Sepsis Model, in Zak et al. (eds.) HANDBOOK OF ANIMAL MODELS OF INFECTION 127-37 (Academic Press 1999).

Male Balb-C mice (6 weeks old, approximately 20 g) were used in the study. Staphylococcus aureus MRSA bacteria (ATCC 33591) were grown at 37° C. with agitation for 12 hours to a stationary phase. Cells were collected by centrifugation, washed twice with saline and resuspended to an O.D.₆₅₀ of 1.4. This suspension was diluted ten times with saline and resuspended to a concentration of 3-5×10⁷ cfu/ml. For each peptide five groups of eight mice per group were infected I.P. with 0.5 ml of the above Staphylococcus aureus MRSA preparation (lethal dose). Forty-five minutes to an hour after infection, each group was treated with a different dose of peptide and a control group received vehicle only. Mice were monitored for fourteen days; death was defined as the end point. Median protecting doses (PD₅₀) were calculated following the Reed and Muench method. See V. Lorian, Antibiotics in Laboratory Medicine, Williams and Wilkins, Baltimore 1991. For IV and SQ models peptide was administered immediately after IP infection.

Toxicity Studies

Peptides were tested for toxicity in vivo using procedures similar to those described previously. See V. Lorian, Antibiotics in Laboratory Medicine, Williams and Wilkins, Baltimore 1991. Mice (male Balb-C) in groups of 4-8 were given via IV, IP or SQ single bolucdoses of peptides and monitored for 14 days. Toxicity of sublethal doses was assessed based on the behavior and appearance of the mice after peptide administration compared to control mice receiving only vehicle. Signs of acute toxicity included lack of activity, red feet and tail, faster breathing. Death was defined as the end point for lethal doses of peptides.

Pathology Studies

Pathology effects of peptides cyclo[RRKWLWLW]-HCl and cyclo-[KQRWLWLW]-HCl were accessed with Balb-C (male 20-25 g). Peptides were administered IP at a lethal dose of 75 mg/kg in 9% sucrose, along with control mice that received vehicle alone. After 50-60 min (in case of cyclo[KQRWLWLW]-HCl), and on the next day (in case of cyclo[RRKWLWLW]-HCl) mice were sacrificed and analyzed (Dr. Osborn, Vet. Pathologist, Department of Animal Resources, TSRI). Pathology studies included blood cell count, and histology examination of different tissues and organs.

Toxicity of multiple dosing was also tested with peptide cyclo[KSKWLWLW]-HCl. Mice (male CD-1,Charles River labs, 20-25 g) were used for this study. A regiment of 200 mg/kg per day of the peptide solution in 9% sucrose for ten consecutive days was administered to 3 mice IP, along with 2 control mice that received vehicle alone. On day eleven mice were sacrificed and analyzed (Dr. Osborn, Vet. Pathologist, Department of Animal Resources, TSRI). Pathology studies included blood cell count, and histology examination of different tissues and organs.

Pharmacokinetics

Pharmacokinetics were performed using methods similar to those described previously. See V. Lorian, Antibiotics in Laboratory Medicine, Williams and Wilkins, Baltimore 1991; W. A. Ritschel, G. L. Kearns Handbook of Basic Pharmacokinetics, American Pharmaceutical Assoc. 5^{th} edition, Washington, D.C. 1999.

Pharmacokinetics Studies Using c[RRKWLWLW]HCl

IV injection: Solution of peptide in 9% sucrose (1 mg/mL) was injected IV into the tail vein of Balb/C mice at a dose of 3.6 mg/kg. Then the blood was collected immediately from one group of mice (3 mice per group) by bleeding the tail, separately from each mouse (50-100 uL per mouse). Further collections of blood were done from other groups of mice at times 20, 40, 70, 90, 120, 180, and 260 min after injection. Plasma from each blood sample was separated immediately after collection by spinning down red blood cells over a period of 5 min at 4000 rev./min. The plasma was then diluted with an equal volume of saline and refrigerated until analysis. Storage of samples under these conditions over a period of 1 month did not change the concentration of peptide.

IP injection: Solutions of peptide in 9% sucrose (8.1 mg/mL) were injected EP into Balb/C mice at a dose of 100 mg/kg. Blood was collected immediately from one group of mice (3 mice per group) by bleeding the tail, separately from each mouse (50-100 uL per mouse). Further collections of blood were done from other groups of mice at times 0.5, 1, 2, 4, 6, 10, 15 h after injection. Plasma from each blood sample was separated immediately after collection by spinning down red blood cells over a period of 5 min at 4000 rev./min. Plasma was then diluted with equal volume of saline and refrigerated until analysis. Storage of samples under these conditions over a period of 1 month did not change the concentration of peptide.

Detection of cyclo[RRKWLWW]-HCl in plasma by HPLC: Diluted with saline plasma (50-100 uL) was added to an eluent A (0.1% HCl in 99% H₂O/1% ACN (v/v)) (150-300 uL), vortexed, and partial precipitation was removed by centrifugation. The clear solution was injected into an HPLC and the peptide was detected at 280 nm, in the 8-10 min interval, using a gradient of eluent A (0.1% HCl in 99% H₂O/1% ACN (v/v)) and eluent B (0.07% HCl in 10% H₂O/90% ACN (v/v)) using a flow rate of 1.5 ml/min. The following gradient was used: 30 to 30% B (5 min), followed by 30 to 37% B (5 min), followed by 37 to 40% B (12.5 min).

Pharmacokinetics Studies Using c[KSKWLWLW]HCl

IV injection: Solution of peptide in 9% sucrose (2 mg/mL) was injected IV into the tail vein of Balb/C mice at a dose of 5 mg/kg. Blood was then collected immediately from one group of mice (3 mice per group) by bleeding the tail, separately from each mouse (50-100 uL per mouse). Further collections of blood were done from other groups of mice at times 30, 60, 120, 230, and 300 min after injection. Plasma from each blood sample was separated immediately after collection by spinning down red blood cells over a period of 5 min at 4000 rev./min. Plasma was then diluted with an equal volume of saline and refrigerated until analysis. Storage of samples under these conditions over a period of 1 month did not change the concentration of peptide.

IP injection: Solution of peptide in 9% sucrose (9.8 mg/mL) was injected IP into Balb/C mice at a dose of 100 mg/kg. Blood was then collected immediately from one group of mice (3 mice per group) by bleeding the tail, separately from each mouse (50-100 uL per mouse). Further collections of blood were done from other groups of mice at times 0.5, 1, 2, 4, 6, 10 h after injection. Plasma from each blood sample was separated immediately after collection by spinning down red blood cells over a period of 5 min at 4000 rev./min. Plasma was then diluted with an equal volume of saline and refrigerated until analysis. Storage at these conditions over a period of 1 month did not change the concentration of peptide.

Detection of c(KSKWLWW) HC in plasma by HPLC: Samples were diluted with saline plasma (50-100 uL), added to an equal volume of eluent A (0.1% TFA in 96.5% H₂O/0.9% ACN/2.4% MeOH (v/v)), vortexed, and partial precipitation was removed by centrifugation. The clear solution was injected into HPLC and the peptide was detected at 280 nm, in the 18-20 min interval, using a gradient of eluent A (0.1% TFA in 96.5% H₂O/0.9% ACN/2.4% MeOH (v/v)) and eluent B (0.05% TFA in 8% H₂O/72% ACN/20% MeOH (v/v)) using a flow rate of 1.5 ml/min. The following gradient was used: 0 to 0% B (5 min), followed by 0 to 100% B (25 min).

Calculation of pharmacokinetic parameters: The area of HPLC peaks corresponding to the peptide in analytical plasma runs was correlated to those of the calibration injections with known amounts of peptide, and the determined concentration was plotted against the time of blood collection. In the first approximation the curve obtained from IV injection fits to the first order kinetic equation (1), indicating a single compartment model. See V. Lorian, Antibiotics in Laboratory Medicine, Williams and Wilkins, Baltimore 1991; W. A. Ritschel, G. L. Kearns Handbook of Basic Pharmacokinetics, American Pharmaceutical Assoc. 5^{th} edition, Washington, D.C. 1999.
Cₜ=C₀*e^{-Kel*t} (1)

The best fit of equation (1) to the experimental points was calculated using SigmaPlot. The following parameters were calculated from the best fit of IV injection curve:
AUC_{IV} (μg*h/mL)—area under the curve for IV injections
C₀ (μg/mL)—concentration of peptide in blood at time zero
Kₑₗ (l/min)—elimination rate constant
T_{1/2} (min)—half life time (time when C=C₀/2); T_{1/2}=(ln2)/Kₑₗ
V (L/kg)—volume of distribution, V=D/C₀, where D is the dose of
   injected peptide expressed in mg/kg
CL (mL/min)—total clearance of peptide was calculated in two ways and
   averaged
CL=Dose/AUC_{IV}, where Dose is total dose of peptide received by
   mouse expressed in μg
CL=Kₑₗ*V*m, where m is mass of the mouse expressed in g
   Equation (2) was used to determine bioavailability (F) of peptide injected via IP route
   F=100%*(AUC_{IP}*D_{IV})/(AUC_{IV}*D_{IP}) (2)
   where AUC_{IP} is area under the concentration vs. time curve from IP injections, D_{IV} and D_{IP} are doses of peptide administered via IV and IP routes respectively, expressed in mg/kg.

### EXAMPLE 2

### In Vitro Antibacterial Activity of Cyclic Peptides

The eight-residue cyclic peptides used in the first experiments of this example had three consecutive polar residues and L-tryptophan and D-leucine repeats that form a hydrophobic surface to promote effective partitioning onto cell membranes. In addition, peptides were most often designed to include at least one basic amino acid residue in order to enhance specificity toward bacterial membranes.

The utility of these design features was demonstrated by in vitro 5 antibacterial assays employing a representative cyclic peptide with sequence Lys-D-Gln-Arg-D-Trp-Leu-D-Trp-Leu-D-Trp (SEQ ID NO:9). Cyclic peptide SEQ ID NO:9 displayed potent activity against gram positive Bacillus subtilis and Staphylococcus aureus and against gram negative Streptococcus pneumonieae and vancomycin-resistant Enterococcus faecalis (Table 4).

TABLE 4

In vitro activity of Lys-D-Gln-Arg-D-
Trp-Leu-D-Trp-Leu-D-Trp (SEQ ID NO: 9)

| Gram positive bacteria | MIC^{a} (μg/ml) |
|---|---|
| Bacillus subtilis^{c} | 3^{b} |
| Bacillus cereus^{d} | 2 |
| Staphylococcus aureus^{e} | 3^{b} |
| Listeria monocytogenes^{f} | 20 |
| Enterococcus faecalis^{g} | 10^{b} |
| Streptococcus pneumoniea^{h} | 10^{b} |
| Gram negative bacteria | MIC (μg/ml) |
| Salmonella typhimuriumⁱ | >70 |
| Mammalian cells | |
| Red blood cell (mouse) | 45^{j} |
| M21 (melanoma) | >75^{k} |

^{a}Minimum inhibitory concentration;
^{b}Data from MDS Panlabs Pharmacology Services;
^{c}ATCC 43223;
^{d}ATCC 11778;
^{e}ATCC25923;
^{f}ATCC19115;
^{g}vancomycin-resistant clinical isolate;
^{h}erythromycin- and ampicillin-resistant clinical isolate;
ⁱATCC 14028;
^{j}Hemolytic dose where 50% of red blood cells are hemolyzed (HD₅₀ (μg/ml))
^{k}LD₅₀ (μg/ml).

A series of six- and eight-residue amphiphilic cyclic peptides were made to further probe the antibacterial activity of these peptides as antibacterial agents and to examine the relationship between surface properties, antibacterial activities, and membrane selectivity. Peptides were synthesized by standard solid-phase BOC or FMOC synthetic protocols, cyclized in solution or on solid support, purified by RP-HPLC, and characterized by MALDI-TOF or ESI-mass spectrometry. The sequences of these peptides are provided in Table 5. Shorthand representation of cyclic peptide sequences using single letter codes was used to allow facile sequence comparisons. Underlining indicates that amino acid is a D-amino acid residue.

TABLE 5

Peptide Sequences

| | | Sequence | Xaa |
|---|---|---|---|
| SEQ ID NO: | Shorthand | Listing | Definition |
| 1-cyclic | QAQAQAQAQAQA | Gln-Xaa₁-Gln- | Xaa₁ = D-Ala |
| | | Xaa₁-Gln-Xaa₁- | |
| | | Gln-Xaa₁-Gln | |
| | | Xaa₁-Gln-Xaa₁ | |
| 2-cyclic | QLWLWLWL | Gln-Xaa₁-Trp- | Xaa₁ = D-Leu |
| | | Xaa₁-Trp-Xaa₁- | |
| | | Trp-Xaa₁ | |
| 3-cyclic | KLWLWLWL | Lys-Xaa₁-Trp- | Xaa₁ = D-Leu |
| | | Xaa₁-Trp-Xaa₁- | |
| | | Trp-Xaa₁ | |
| 4-cyclic | KLKLKLKL | Lys-Xaa₁-Lys- | Xaa₁ = D-Leu |
| | | Xaa₁-Lys-Xaa₁- | |
| | | Lys-Xaa₁ | |
| 5-cyclic | SKSWLWLW | Xaa₁-Lys-Xaa₁- | Xaa₁ = D-Ser |
| | | Trp-Xaa₂-Trp- | Xaa₂ = D-Leu |
| | | Xaa₂-Trp | |
| 6-cyclic | THSWLWLW | Xaa₁-His-Xaa₂- | Xaa₁ = D-Thr |
| | | Trp-Xaa₃-Trp- | Xaa₂ = D-Ser |
| | | Xaa₃-Trp | Xaa₃ = D-Leu |
| 7-cyclic | RGDWLWLW | Xaa₁-Gly-Xaa₂- | Xaa₁ = D-Arg |
| | | Trp-Xaa₃-Trp- | Xaa₂ = D-Asp |
| | | Xaa₃-Trp | Xaa₃ = D-Leu |
| 8-cyclic | KQRWLWLW | Xaa₁-Gln-Xaa₂- | Xaa₁ = D-Lys |
| | | Trp-Xaa₃-Trp- | Xaa₂ = D-Arg |
| | | Xaa₃-Trp | Xaa₃ = D-Leu |
| 9-cyclic | KQRWLWLW | Lys-Xaa₁-Arg- | Xaa₁ = D-Gln |
| | | Xaa₂-Leu-Xaa₂- | Xaa₂ = D-Trp |
| | | Leu-Xaa₃ | |
| 10-cyclic | RQRWLWLW | Xaa₁-Gln-Xaa₁- | Xaa₁ = D-Arg |
| | | Trp-Xaa₂-Trp- | Xaa₂ = D-Leu |
| | | Xaa₂-Trp | |
| 11-cyclic | KQKWLWLW | Xaa₁-Gln-Xaa₁- | Xaa₁ = D-Lys |
| | | Trp-Xaa₂-Trp- | Xaa₂ = D-Leu |
| | | Xaa₂-Trp | |
| 12-cyclic | KSKWLWLW | Xaa₁-Ser-Xaa₁- | Xaa₁ = D-Lys |
| | | Trp-Xaa₂-Trp- | Xaa₂ = D-Leu |
| | | Xaa₂-Trp | |
| 13-cyclic | SHKWLWLW | Xaa₁-His-Xaa₂- | Xaa₁ = D-Ser |
| | | Trp-Xaa₃-Trp- | Xaa₂ = D-Lys |
| | | Xaa₃-Trp | Xaa₃ = D-Leu |
| 14-cyclic | SKHWLWLW | Xaa₁-Lys-Xaa₂- | Xaa₁ = D-Ser |
| | | Trp-Xaa₃-Trp- | Xaa₂ = D-His |
| | | Xaa₃-Trp | Xaa₃ = D-Leu |
| 15-cyclic | SHHWLWLW | Xaa₁-His-Xaa₂- | Xaa₁ = D-Ser |
| | | Trp-Xaa₃-Trp- | Xaa₂ = D-His |
| | | Xaa₃-Trp | Xaa₃ = D-Leu |
| 16-cyclic | EKHWLWLW | Xaa₁-Lys-Xaa₂- | Xaa₁ = D-Glu |
| | | Trp-Xaa₃-Trp- | Xaa₂ = D-His |
| | | Xaa₃-Trp | Xaa₃ = D-Leu |
| 17-cyclic | KKKWLWLW | Xaa₁-Lys-Xaa₁- | Xaa₁ = D-Lys |
| | | Trp-Xaa₂-Trp- | Xaa₂ = D-Leu |
| | | Xaa₂-Trp | |
| 18-cyclic | RRKWLWLW | Xaa₁-Arg-Xaa₂- | Xaa₁ = D-Arg |
| | | Trp-Xaa₃-Trp- | Xaa₂ = D-Lys |
| | | Xaa₃-Trp | Xaa₃ = D-Leu |
| 19-cyclic | KRKWLWLW | Xaa₁-Arg-Xaa₁- | Xaa₁ = D-Lys |
| | | Trp-Xaa₂-Trp- | Xaa₂ = D-Leu |
| | | Xaa₂-Trp | |
| 20-cyclic | RRRWLWLW | Xaa₁-Arg-Xaa₁- | Xaa₁ = D-Arg |
| | | Trp-Xaa₂-Trp- | Xaa₂ = D-Leu |
| | | Xaa₂-Trp | |
| 21-cyclic | HKHWLWLW | Xaa₁-Lys-Xaa₁- | Xaa₁ = D-His |
| | | Trp-Xaa₂-Trp- | Xaa₂ = D-Leu |
| | | Xaa₂-Trp | |
| 22-cyclic | KHKWLWLW | Xaa₁-His-Xaa₁- | Xaa₁ = D-Lys |
| | | Trp-Xaa₂-Trp- | Xaa₂ = D-Leu |
| | | Xaa₂-Trp | |
| 23-linear | WKKKWLWLW | Trp-Xaa₁-Lys- | Xaa₁ = D-Lys |
| | | Xaa₁-Trp-Xaa₂- | Xaa₂ = D-Leu |
| | | Trp-Xaa₂-Trp | |
| 24-linear | KKKWLWLW | Xaa₁-Lys-Xaa₁- | Xaa₁ = D-Lys |
| | | Trp-Xaa₂-Trp- | Xaa₂ = D-Leu |
| | | Xaa₂-Trp | |
| 25-linear | Ac-KKKWLWLW- | Xaa₁-Lys-Xaa₂- | Xaa₁ = Ac-D- |
| | CONH₂ | Trp-Xaa₃-Trp- | Lys |
| | | Xaa₃-Xaa₄ | Xaa₂ = D-Lys |
| | | | Xaa₃ = D-Leu |
| | | | Xaa₄ = D- |
| | | | Trp-CONH₂ |
| 26-cyclic | KKLWLW | Xaa₁-Lys-Xaa₂- | Xaa₁ = D-Lys |
| | | Trp-Xaa₂-Trp | Xaa₂ = D-Leu |
| 27-cyclic | KHLWLW | Xaa₁-His-Xaa₂- | Xaa₁ = D-Lys |
| | | Trp-Xaa₂-Trp | Xaa₂ = D-Leu |
| 28-cyclic | KSLWLW | Xaa₁-Ser-Xaa₂- | Xaa₁ = D-Lys |
| | | Trp-Xaa₂-Trp | Xaa₂ = D-Leu |
| 29-cyclic | RRLWLW | Xaa₁-Arg-Xaa₂- | Xaa₁ = D-Arg |
| | | Trp-Xaa₂-Trp | Xaa₂ = D-Leu |
| 30-cyclic | QRUUUFWR | Glu-Xaa₁-Xaa₂- | Xaa₁ = D-Arg |
| | | Xaa₃-Xaa₂- | Xaa₂ = L- |
| | | Xaa₃-Trp-Xaa₁ | hydrophobic |
| | | | Xaa₃ = D- |
| | | | hydrophobic |
| | | | Xaa₄ = D-Phe |
| 31-cyclic | QRUUUWWR | Glu-Xaa₁-Xaa₂- | Xaa₁ = D-Arg |
| | | Xaa₃-Xaa₂- | Xaa₂ = L- |
| | | Xaa₃-Trp-Xaa₁ | hydrophobic |
| | | | Xaa₃ = D- |
| | | | hydrophobic |
| | | | Xaa₄ = D-Trp |
| 32-cyclic | QRUUUAWR | Glu-Xaa₁-Xaa₂- | Xaa₁ = D-Arg |
| | | Xaa₃-Xaa₂- | Xaa₂ = L- |
| | | Xaa₃-Trp-Xaa₁ | hydrophobic |
| | | | Xaa₃ = D- |
| | | | hydrophobic |
| | | | Xaa₄ = D-Ala |
| 33-cyclic | QRUUULWR | Glu-Xaa₁-Xaa₂- | Xaa₁ = D-Arg |
| | | Xaa₃-Xaa₂- | Xaa₂ = L- |
| | | Xaa₃-Trp-Xaa₁ | hydrophobic |
| | | | Xaa₃ = D- |
| | | | hydrophobic |
| | | | Xaa₄ = D-Leu |
| 34-cyclic | QRUUFWWR | Glu-Xaa₁-Xaa₂- | Xaa₁ = D-Arg |
| | | Xaa₃-Phe-Xaa₄- | Xaa₂ = L- |
| | | Trp-Xaa₁ | hydrophobic |
| | | | Xaa₃ = D- |
| | | | hydrophobic |
| | | | Xaa₄ = D-Trp |
| 35-cyclic | QRUWLWWR | Glu-Xaa₁-Xaa₂- | Xaa₁ = D-Arg |
| | | Xaa₃-Leu-Xaa₃- | Xaa₂ = L- |
| | | Trp-Xaa₁ | hydrophobic |
| | | | Xaa₃ = D-Trp |
| 36-cyclic | QRWWLWWR | Glu-Xaa₁-Trp- | Xaa₁ = D-Arg |
| | | Xaa₂-Leu-Xaa₂- | Xaa₂ = D-Trp |
| | | Trp-Xaa₁ | |
| 37-cyclic | QRUUWWWR | Glu-Xaa₁-Xaa₂- | Xaa₁ = D-Arg |
| | | Xaa₃-Trp-Xaa₄- | Xaa₂ = L- |
| | | Trp-Xaa₁ | hydrophobic |
| | | | Xaa₃ = D- |
| | | | hydrophobic |
| | | | Xaa₄ = D-Trp |
| 38-cyclic | QRVWLWWR | Glu-Xaa₁-Val- | Xaa₁ = D-Arg |
| | | Xaa₂-Leu-Xaa₂- | Xaa₂ = D-Trp |
| | | Trp-Xaa₁ | |
| 39-cyclic | QRFWLWWR | Glu-Xaa₁-Phe- | Xaa₁ = D-Arg |
| | | Xaa₂-Leu-Xaa₂- | Xaa₂ = D-Trp |
| | | Trp-Xaa₁ | |
| 142-cyclic | QRWWLWWR | Glu-Xaa₁-Trp- | Xaa₁ = D-Arg |
| | | Xaa₂-Leu-Xaa₂- | Xaa₂ = D-Trp |
| | | Trp-Xaa₁ | |

Antibacterial activities were tested against E. coli and methicillin-resistant Staphylococcus aureus (MRSA)—currently the major component of approximately two million annual nosocomial infections in the United States (Background and History of Emerging Staphylococcus aureus strains with reduced susceptibility to vancomycin, website at narsaweb.narsa.net). Antibacterial assays were performed as described above using standard microdilution methods. Results are provided in Tables 6, 7, 8 and 9.

TABLE 6

Antibacterial Activity of
Peptides Containing D-, L-α-Amino Acids

| | | S. aureus | | |
|---|---|---|---|---|
| SEQ | Shorthand | (MRSA)^{a} | E. coli^{b} | Hemolysis |
| ID NO: | Sequence^{c} | MIC μg/ml | MIC μg/ml | HD50 μg/ml |
| 5- | SKSWLWLW | 10 | 100 | 30 |
| cyclic | | | | |
| 6- | THSWLWLW | 100 | 100 | 100 |
| cyclic | | | | |
| 7- | RGDWLWLW | 100 | 100 | 100 |
| cyclic | | | | |
| 8- | KQRWLWLW | 6 | 80 | 45 |
| cyclic | | | | |
| 9- | KQRWLWLW | 8 | 80 | 40 |
| cyclic | | | | |
| 10- | RQRWLWLW | 18 | 90 | 25 |
| cyclic | | | | |
| 11- | KQKWLWLW | 45 | 100 | 50 |
| cyclic | | | | |
| 12- | KSKWLWLW | 5 | 40 | 100 |
| cyclic | | | | |
| 13- | SHKWLWLW | 10 | 100 | 35 |
| cyclic | | | | |
| 14- | SKHWLWLW | 15 | 100 | 20 |
| cyclic | | | | |
| 15- | SHHWLWLW | 20 | 100 | 20 |
| cyclic | | | | |
| 16- | EKHWLWLW | 100 | 100 | >100 |
| cyclic | | | | |
| 17- | KKKWLWLW | 8 | 70 | 50 |
| cyclic | | | | |
| 18- | RRKWLWLW | 6 | 15 | 50 |
| cyclic | | | | |
| 19- | KRKWLWLW | 10 | 40 | 50 |
| cyclic | | | | |
| 20- | RRRWLWLW | 10 | 50 | 35 |
| cyclic | | | | |
| 21- | HKHWLWLW | 12 | 15 | 25 |
| cyclic | | | | |
| 22- | KHKWLWLW | 10 | 80 | 30 |
| cyclic | | | | |
| 23- | WKKKWLWLW | 60 | | |
| linear | | | | |
| 24- | KKKWLWLW | 50 | | |
| linear | | | | |
| 25- | Ac-KKKWLWLW- | 80 | | |
| linear | CONH2 | | | |
| 26- | KKLWLW | 10 | 17 | 80 |
| cyclic | | | | |
| 27- | KHLWLW | 10 | 100 | 25 |
| cyclic | | | | |
| 28- | KSLWLW | 75 | 100 | 90 |
| cyclic | | | | |
| 29- | RRLWLW | 35 | 5 | 90 |
| cyclic | | | | |
| 30- | QRUUUFWR^{d} | | | |
| cyclic | | | | |
| 31- | QRUUUWWR^{d} | | | |
| cyclic | | | | |
| 32- | QRUUUAWR | | 37 | |
| cyclic | | | | |
| 33- | QRUUULWR | | | |
| cyclic | | | | |
| 34- | QRUUFWWR^{d} | 2.5-3.8 | | |
| cyclic | | | | |
| 35- | QRUWLWWR^{d} | | | |
| cyclic | | | | |
| 36- | QRWWLWWR^{d} | 2.5-3.8 | | |
| cyclic | | | | |
| 37- | QRUUWWWR | 2.5-3.8 | | |
| cyclic | | | | |
| 38- | QRVWLWWR | 2.5-3.8 | | |
| cyclic | | | | |
| 39- | QRFWLWWR | 2.5-3.8 | | |
| cyclic | | | | |
| 142- | QRWWLWWR | 2.5-3.8 | | |
| cyclic | | | | |

^{a}ATCC 33591.
^{b}JM109(DE3).
^{c}U is a hydrophobic amino acid selected from the group consisting of alanine, leucine, valine, phenylalanine and tryptophan.
^{d}Activity against Bacillus subtilis at about 1.7 to 2.5 μg/ml.
Peptide mixtures SEQ ID NO: 30-37 are complex peptide mixtures and their concentrations for the anti-bacterial assay could not be specifically determined. The MIC values for these sequences may differ by up to five-fold.

The role of polar side chains in bioactivity and membrane selectivity may be evaluated upon examination of the antibacterial activities listed in Table 6.

Cyclic peptides with SEQ ID NO:5 and 6 each bear one basic residue between two serine residues, or between a serine and a threonine residue, respectively. While peptide SEQ ID NO:5 [cyclo-(D-Ser-Lys-D-Ser-Trp-D-Leu-Trp-D-Leu-Trp)] displays good activity against S. aureus MRSA, substitution of lysine by histidine in peptide SEQ ID NO:6 [cyclo-(D-Thr-His-D-Ser-Trp-D-Leu-Trp-D-Leu-Trp)] significantly decreases the activity.

Cyclic peptides with SEQ ID NO:8-15 each possess two basic amino acids and one neutral, polar amino acid. These cyclic peptides vary in antibacterial activity and in red blood cell hemolysis. A single glutamic acid substitution has a deleterious effect on the antibacterial activity as indicated by the large difference in activity against S. aureus MRSA bacteria between peptide SEQ ID NOs:14 [cyclo-(D-Ser-Lys-D-His-Trp-D-Leu-Trp-D-Leu-Trp)] and 16 [cyclo-(D-Glu-Lys-D-His-Trp-D-Leu-Trp-D-Leu-Trp)]. The reduced activity of the glutamic acid-containing peptide may be due to unfavorable electrostatic interactions of the carboxylate side chain with bacterial membrane constituents.

Increasing the number of basic residues from two to three in peptide SEQ ID NOs:17-22 provided high activities against S. aureus MRSA. Peptides with SEQ ID NO:18 [cyclo-(D-Arg-Arg-D-Lys-Trp-D-Leu-Trp-D-Leu-Trp)] and SEQ ID NO:21 [cyclo-(D-His-Lys-D-His-Trp-D-Leu-Trp-D-Leu-Trp)] also exhibited moderate activities against E. coli. The in vitro antibacterial activities of hexameric peptides with SEQ ID NOs:26-29 also indicate that use of basic amino acids in the cyclic peptides may increase antibacterial activity and improve selectivity for bacterial membranes. Peptide SEQ ID NO:26 [cyclo-(D-Lys-Lys-D-Leu-Trp-D-Leu-Trp)], with two consecutive lysine residues, exhibits broad-spectrum activity and has low hemolytic properties. On the other hand, peptide SEQ ID NO:27 [cyclo-(D-Lys-His-D-Leu-Trp-D-Leu-Trp)], which has a histidine instead of the lysine in peptide SEQ ID NO:26, retains high activity against S. aureus MRSA but is inactive against E. coli. Substituting a lysine in peptide SEQ ID NO:26 with serine yields the less active peptide SEQ ID NO:28 [cyclo-(D-Lys-Ser-D-Leu-Trp-D-Leu-Trp)]. However, peptide SEQ ID NO:29 [cyclo-(D-Arg-Arg-D-Leu-Trp-D-Leu-Trp)], which possesses two arginine residues, displays potent and selective activity against E. coli with low levels of hemolysis.

The spectrum of activity and membrane selectivity observed with the above peptides indicates that even single amino acid substitutions can influence antibacterial activity and selectivity versus mammalian cells.

The effect of plasma proteins on the availability and stability of cyclic peptides in vitro was also examined. Antibacterial activities remained unchanged in the presence of large amounts (up to 50% v/v) of fetal bovine serum (FBS) in the culture media. However, most peptides examined have a reduced level of hemolytic activity in the presence of 5-10% FBS in the assay mixture, compared to analogous assays in the absence of FBS. For example, in the presence of 10% FBS, peptide SEQ ID NO:8 [cyclo-(D-Lys-Gln-D-Arg-Trp-D-Leu-Trp-D-Leu-Trp)] displays five-fold reduction in hemolytic activity (from HD₅₀=10 to 50 μg/ml).

Cyclic peptides having SEQ ID NO:13, 18, 26, and 29 were assayed for proteolytic susceptibility. The peptides show abiotic structure and conformational preferences, and were stable in the presence of trypsin, α-chymotrypsin, subtilisin, and blood plasma. No significant peptide degradation was observed in chromatograms obtained from RP-HPLC over a 24 h time period, whereas control linear L-α-amino acid peptides were degraded in less than 10 min under similar reaction conditions and within four hours when placed in murine blood plasma.

Results for further testing of gram-negative and gram-positive bacteria are shown in Tables 7 and 8 respectively, along with control assays using FDA approved antibiotics.

The shorthand representation of cyclic peptide sequences was utilized where single amino acid letter codes are employed to facilitate sequence comparisons. Underlining indicates that amino acid is a D-amino acid residue and brackets indicate that the peptides are circular.

TABLE 7

Gram Negative Bacteria

| | | | |
|---|---|---|---|
| | | E. coli | H. |
| | E. coli | (EL744) K12 | influenzae |
| | (K12) | (tolC::kan) | ATCC 49247 |
| | MHII broth | MHII broth | HTM |
| Peptide Sequence | MIC (ug/mL) | MIC (ug/mL) | MIC (ug/mL) |

| | | | |
|---|---|---|---|
| [KKLWLW]-HCl | >128 | 32 | >128 |
| [KSKWLWLW]-HCl | >128 | >128 | 32 |
| [RRLWLW]-HCl | 16 | 8 | 8-16 |
| [RRKWLWLW]-HCl | 32 | 16 | 32-64 |
| Kanamycin | 4 | >128 | 4 |
| Norfloxacin | 0.06 | 0.016 | 0.06 |
| Penicillin G | 32 | 16 | 4 |
| Vancomycin | 128 | 128 | 128 |

TABLE 8

Gram-Positive Bacteria

| | | | |
|---|---|---|---|
| | E. faecalis | E. faecium | S. aureus |
| | ATCC 51299 | SP180 | ATCC 33592 |
| | BHI broth | BHI broth | MHII broth |
| Peptide Sequence | MIC (ug/mL) | MIC (ug/mL) | MIC (ug/mL) |

| | | | |
|---|---|---|---|
| [KKLWLW]-HCl | >128 | 16 | 32 |
| [KSKWLWLW]-HCl | 8 | 4 | 4 |
| [RRLWLW]-HCl | 32-64 | 8-16 | 32 |
| [RRKWLWLW]-HCl | 8-16 | 4 | 8 |
| Kanamycin | >128 | >128 | >128 |
| Norfloxacin | 2 | 32 | 0.5 |
| Penicillin G | 0.5 | 128 | 64 |
| Vancomycin | 32 | >128 | 0.5 |

The antibacterial properties of different types of cyclic peptides of the invention are further illustrated in Table 9. Each cyclic peptide is described in shorthand using underlining to indicate which amino acids have D-chirality, brackets to identify cyclic peptides and a number in parentheses to indicate the SEQ ID NO: (i.e., NO:). These peptides were tested against methicilin-resistant Staphyococus aureus (MRSA)(ATCC 33591) and vancomycin-resistant Enterococus faecalis (VRE)(ATCC 51575) and for hemolytic activity against mammalian red blood cells.

TABLE 9

In Vitro Activity Against Methicilin-
Resistant Staphyococus aureus (MRSA) and
Vancomycin-Resistant Enterococus faecalis (VRE)

| | | | | |
|---|---|---|---|---|
| | | | RBC | |
| | | | Hemolysis | Media |
| Peptide sequence | MRSA | VRE | (HD₅₀) | Used |

| | | | | |
|---|---|---|---|---|
| Control Antibiotics | | | | |
| KANAMYCIN | 150 | >600 | >600 | b, c |
| MELLITIN | 6.3 | 6.3 | 10 | b, c |
| NORFLOXACIN | <2.8 | <2.8 | >360 | b, c |
| VANCOMYCIN <1.8 | >240 | >240 | b, c | |
| Group-I octamers | | | | |
| [KFWLWLWF] (40) | 33 | >44 | >44 | a, a |
| [KLWLWLWL] (41) | 12 | 25 | 30 | b, c |
| Group-IL octamers | | | | |
| [KSWLWLWF] (42) | >26 | >26 | >26 | a, a |
| [KWWLWLWH] (43) | 13 | 17 | >23 | a, a |
| [KWWLWLKW] (44) | 4 | 4 | >58 | a |
| Group-III octamers | | | | |
| [EKHWLWLW] (16) | 100 | 100 | >100 | a, a |
| [HKHWLWLW] (21) | 12 | 16 | 25 | a, a |
| [KHKLALAL] (45) | 50 | 200 | >200 | b, c |
| [KHKLALWL] (46) | 25 | 50 | >200 | b, c |
| [KHKLFLAL] (47) | 3 | 13 | >220 | b, c |
| [KHKLFLFL] (48) | 7 | 13 | >220 | b, c |
| [KHKLFLWL] (49) | 12 | 25 | 200 | b, c |
| [KHKLWLWL] (50) | 9 | 18 | 150 | b, c |
| [KHKWLWLW] (22) | 10 | 10 | 30 | a, a |
| [KKKFLFLF] (51) | 18 | 75 | >300 | b, c |
| [KKKWLWLW] (17) | 10 | 40 | 45 | a, a |
| [KKKWLWLW-HCl] (17) | 6 | 6 | 75 | b, c |
| [KQRWLWLW] (9) | 12 | 12 | 40 | a, a |
| [KQRWLWLW-HCl] (8) | 8 | 8 | 70 | b, c |
| [KRKLFLFL] (52) | 11 | 22 | >180 | b, c |
| [KRKWLWLW] (19) | 15 | 8 | 50 | a, a |
| [KSKLALAL] (53) | 35 | >280 | >280 | b, c |
| [KSKLFLFL] (54) | 50 | 200 | >200 | b, c |
| [KSKLFLFL] (55) | 200 | 50 | >200 | b, b |
| [KSKLGLGL] (56) | >240 | >240 | >240 | b, c |
| [KSKLILIL] (57) | 200 | >200 | >200 | b, c |
| [KSKLILIL] (58) | >200 | 50 | >200 | b, b |
| [KSKLNleLNleL] (59) | 220 | >200 | >220 | b, c |
| [KSKLVLVL] (60) | >220 | >220 | >220 | b, c |
| [KSKLWLWL] (61) | 15 | 15 | 125 | b, c |
| [KSKLYLYL] (62) | 100 | 200 | >200 | b, c |
| [KSKWLWLW] (12) | 5 | 15 | 100 | a, a |
| [KSKWLWLW-HCl] (12) | 9 | 9 | 60 | b, c |
| [KSOLWLWL] (63) | 12 | 25 | 150 | b, c |
| [KTKLALAL] (64) | 80 | >160 | >160 | b, c |
| [KTKLFLFL] (65) | 60 | >240 | >240 | b, c |
| [KTKLWLWL] (66) | 18 | 18 | 150 | b, c |
| [KWWLWLKS] (67) | 7 | 10 | >153 | b, b |
| [QRFWLWWR] (68) | 7.5 | 3.8 | 60 | b, b |
| [QRLWLWWR] (69) | 6.3 | 6.3 | 35 | b, b |
| [QRVWLWWR] (70) | 12.5 | 6.3 | 30 | b, b |
| [QRWWLWWR] (71) | 6.3 | 6.3 | 35 | b, b |
| [RGDWLWLW] (7) | 100 | 100 | 100 | a |
| [RQRWLWLW] (10) | 10 | 10 | 25 | a |
| [RRKWLWLW] (18) | 5 | 5 | 50 | a |
| [RRKWLWLW-HCl] (18) | 9 | 9 | 70 | b, c |
| [SHHWLWLW] (15) | 30 | 50 | 20 | a |
| [SHHWLWLW-HCl] (15) | 6 | 6 | 40 | b, c |
| [SHKWLWLW] (13) | 10 | 40 | 35 | a, a |
| [SKHWLWLW] (14) | 15 | 30 | 20 | a, a |
| [SKSWLWLW] (5) | 20 | 20 | 30 | a, a |
| [THSWLWLW] (6) | 100 | 100 | 100 | a, a |
| Group-IV octamers | | | | |
| [KHKHFLWL] (72) | 8 | 16 | 200 | b, c |
| [KHKHWLWL] (73) | 18 | 18 | 150 | b, c |
| [KRWLWLKH] (74) | 5 | 7 | >130 | a, a |
| [KRWLWLKS] (75) | 9 | 22 | >48 | a, a |
| [KSKKFLFL] (76) | 5 | 10 | 100 | b, c |
| [KSKKWLWL] (77) | 13 | 27 | >220 | b, c |
| [KSSKALAL] (78) | >240 | >240 | >240 | b, c |
| [KSSKFLFL] (79) | 43 | 43 | >200 | b, c |
| [KSSKVLVL] (80) | 22 | 180 | >180 | b, c |
| [KSSKWLWL] (82) | 27 | 27 | >220 | b, c |
| [KSSKYLYL] (83) | 85 | 170 | >340 | b, c |
| [KSWLWLRS] (84) | 9 | 12 | >62 | a |
| Group-V octamers | | | | |
| [KHKHKWLW] (85) | 6 | 6 | 150 | b, c |
| [KHQHKLWL] (86) | 100 | 100 | >200 | b, c |
| [KHQHKWLW] (87) | 6 | 12 | 200 | b, c |
| [KHSHKWLW] (88) | 6 | 6 | 200 | b, c |
| [KNQNKFWF] (89) | 17 | 70 | 280 | b, c |
| [KNQNKLWL] (90) | >200 | >200 | >200 | b, c |
| [KSKSKWLW] (91) | 6 | 12 | 200 | b, c |
| [KSQSKWLW] (92) | 9 | 18 | 195 | b, c |
| [KSSSKWLW] (93) | 6 | 6 | 175 | b, c |
| [KYQYKNWN] (94) | >200 | >200 | >200 | b, c |
| Group-VI octamers | | | | |
| [KAQAKAWA] (95) | >200 | >200 | >200 | b, c |
| [KAQNKAWN] (96) | >200 | >200 | >200 | b, c |
| [KAQFKAWF] (97) | 100 | 100 | >200 | b, c |
| [KFQFKNWN] (98) | >200 | >200 | >280 | b, c |
| [KFWLWLHW] (99) | 21 | 21 | >25 | a |
| [KFWLWLKF] (100) | 11 | 10 | >78 | a |
| [KLKLKLKL] (3) | >200 | >200 | >200 | b, c |
| [KRKWLHLW] (101) | 40 | 40 | 100 | a |
| [KRQFKFWF] (102) | 12 | 12 | >200 | b, c |
| [KSKLHLHL] (103) | >320 | >320 | >320 | b, c |
| [KSKLQLQL] (104) | >200 | >200 | >200 | b, c |
| [KSKLRLRL] (105) | >240 | >240 | >240 | b, c |
| [KSKLRLRL] (106) | >400 | >400 | >400 | b, c |
| [KWKWSWLW] (107) | 12 | 12 | 150 | b, c |
| [KYQYKNWN] (108) | >200 | >200 | >200 | b, c |
| [RRKWLELW] (109) | 80 | 100 | 100 | a |
| [RRKWLHLW] (110) | 25 | 40 | 100 | a |
| [RRKWLKLW] (111) | 12 | 80 | 100 | a |
| [RRKWLSLW] (112) | 12 | 50 | 100 | a |
| Group-II hexamers | | | | |
| [KHLWLW] (27) | 10 | 40 | 25 | a |
| [KKLWLW] (26) | 10 | 40 | 80 | a |
| [KRVWAI] (113) | >200 | >200 | >200 | b, c |
| [KSLWLW] (28) | 100 | 100 | 90 | b, c |
| [KWILAR] (114) | 50 | >200 | >200 | b, c |
| [RRWLWL] (115) | 40 | 100 | 90 | a, a |
| [KWQWLW] (116) | 12 | 12 | >16 | a, a |
| Group-III hexamers | | | | |
| [KEKWLW] (117) | 101 | 91 | >121 | a, a |
| [KEQWLW] (118) | >115 | >115 | >115 | a, a |
| [KHKWLW] (119) | 12 | 60 | >115 | a, a |
| [KHQWLW] (120) | 43 | 57 | 115.0 | a, a |
| [KKKWLW] (121) | 23 | 51 | >111 | a, a |
| [KKQWLW] (122) | 27 | 81 | >215 | a, a |
| [KNQWLW] (123) | >74 | >74 | >74 | a, a |
| [KQQWLW] (124) | >35 | >35 | >35 | a, a |
| [KRKWLW] (125) | 9 | 33 | >174 | a, a |
| [KSKWLW] (126) | 19 | 38 | >65 | a, a |
| [KSYVWS] (127) | >200 | >200 | >200 | b, c |
| Group-VI hexamers | | | | |
| [KFKWLW] (128) | 14 | 43 | >61 | a, a |
| [KFQWLW] (129) | 25 | 10 | >81 | a, a |
| [KHQSRT] (130) | >200 | >200 | >200 | b, c |
| [KHQSRT] (131) | >200 | >200 | >200 | a, a |
| [KSIRIT] (132) | >200 | >200 | 175 | b, c |

All peptides tested were TFA salts except where noted. Media “a” was anti-microbial media-3 (AM-3). Media “b” was cation adjusted Meuller Hinton broth (MHBII). Media “c” was Brain Heart Infusion (BHI).

In another series of experiments (Table 10), the activity of cyclic peptides of the invention was tested against a larger variety of bacterial species. The bacterial species tested were as follows: Vancomycin resistant Enterociccus faecalis (VRE, ATCC 51575); methicilin resistant Staphylococcus aureus (ATCC 33591, MRSA); E. coli: JM109 (DE3) Bacillus cereus (ATCC1 1778); and Streptococcus pneumoniae (ATCC 6301). Murine red blood cells were used for the hemolysis assays as described in Example 1.

TABLE 10

Minimum Inhibitory Concentrations (MIC) in mg/ml

| | | | | | | Hemo- |
|---|---|---|---|---|---|---|
| | | | | | | lysis |
| | | | E. | B. | S. | HD₅₀ |
| Peptide | VRE | MRSA | coli | cerius | pneum. | mg/ml |
| Group-I octamer | | | | | | |
| [KLWLWLWL] | 80 | ppt | 100 | 50 | 50 | 20 |
| (3) | | | | | | |
| Group-II octamer | | | | | | |
| [KELWLWLW] | 100 | 100 | | >140 | 100 | |
| (143) | | | | | | |
| Group-III octamer | | | | | | |
| [KSKWLWLW] | 15 | 5 | 40 | <3 | 10 | 100 |
| (12) | | | | | | |
| [RRKWLWLW] | 5 | 5 | 15 | 5 | 5 | 50 |
| (18) | | | | | | |
| [RQRWLWLW] | 10 | 10 | 100 | <1 | 4 | 25 |
| (10) | | | | | | |
| [SHKWLWLW] | 40 | 10 | 100 | 12 | | 35 |
| (13) | | | | | | |
| [KKKWLWLW] | 40 | 10 | 65 | 5 | 10 | 45 |
| (17) | | | | | | |
| [RRRWLWLW] | | 10 | 35 | 10 | 5 | 35 |
| (133) | | | | | | |
| [KHKWLWLW] | 10 | 10 | 80 | 8 | 8 | 30 |
| (22) | | | | | | |
| [KQRWLWLW] | 12 | 12 | 80 | 3 | 4 | 40 |
| (9) | | | | | | |
| [HKHWLWLW] | 16 | 12 | 15 | 4 | 19 | 25 |
| (21) | | | | | | |
| [SKHWLWLW] | 30 | 15 | 100 | 6 | | 20 |
| (14) | | | | | | |
| [KRKWLWLW] | 8 | 15 | 60 | 3 | 5 | 50 |
| (19) | | | | | | |
| [SKSWLWLW] | 20 | 20 | 100 | >30 | 30 | 30 |
| (5) | | | | | | |
| [KQKWLWLW] | 20 | 20 | 100 | 7 | 8 | 50 |
| (11) | | | | | | |
| [SHHWLWLW] | 50 | 30 | 100 | 11 | 40 | 20 |
| (15) | | | | | | |
| [THSWLWLW] | 100 | 100 | 100 | >280 | 100 | 100 |
| (6) | | | | | | |
| [RGDWLWLW] | 100 | 100 | 100 | >280 | 70 | 100 |
| (7) | | | | | | |
| [EKHWLWLW] | 100 | 100 | 100 | 85 | 50 | >100 |
| (16) | | | | | | |
| Group-VI octamer | | | | | | |
| [RRKWLKLW] | 80 | 12 | 6 | 23 | 25 | 100 |
| (111) | | | | | | |
| [RRKWLSLW] | 50 | 12 | 12 | 13 | 12 | 100 |
| (112) | | | | | | |
| [RRKWLHLW] | 40 | 25 | 15 | 20 | 20 | 100 |
| (110) | | | | | | |
| [KRKWLHLW] | 40 | 40 | 12 | 80 | 80 | 100 |
| (101) | | | | | | |
| [RRKWLELW] | 100 | 80 | 40 | 195 | 100 | 100 |
| (109) | | | | | | |
| linearized | | | | | | |
| octamers | | | | | | |
| H-WKKKWLWL-OH | 100 | 60 | | 340 | 100 | |
| (144) | | | | | | |
| H-KKKWLWLW-NH2 | 100 | 30 | 3 | 19 | 80 | 100 |
| (134) | | | | | | |
| Ac-KKKWLWLW-NH2 | 100 | 100 | 20 | 75 | 80 | 100 |
| (25) | | | | | | |
| H-RRKWLWLW-NH2 | 100 | 20 | 4 | 19 | 40 | 100 |
| (135) | | | | | | |
| Ac-RRKWLWLW-NH2 | 100 | 70 | 6 | 22 | 40 | 100 |
| (136) | | | | | | |
| H-LWRRKWLW-NH2 | 40 | 5 | 4 | 9 | 20 | 100 |
| (137) | | | | | | |
| Ac-LWRRKWLW-NH2 | 100 | 15 | 7 | 20 | 20 | 100 |
| (138) | | | | | | |
| Backbone | | | | | | |
| modified octamers | | | | | | |
| c[RRKW^{Me}LW^{Me}LW] | 30 | 5 | 5 | 5 | 10 | 100 |
| (139) | | | | | | |
| c[KQRW^{Me}LW^{Me}LW] | 100 | 10 | | 8 | 10 | |
| (140) | | | | | | |
| Group-II hexamer | | | | | | |
| [KKLWLW] | 40 | 10 | 17 | >10 | 30 | 80 |
| (26) | | | | | | |
| [KHLWLW] | 40 | 10 | 100 | 5 | 5 | 25 |
| (27) | | | | | | |
| [RKLWLW] | 25 | 25 | | 13 | 20 | |
| (141) | | | | | | |
| [RRWLWL] | 100 | 40 | 5 | 24 | 12 | 90 |
| (115) | | | | | | |
| [KSLWLW] | 100 | 100 | 100 | 15 | 20 | 90 |
| (28) | | | | | | |

The speed at which several of the cyclic peptides of the invention kill E. coli JM 109 (DE3), Enterococcus faecium SP 180 and S. aureus (MRSA) (ATCC No. 33591 and 33592) was further tested at room temperature and/or at 37° C. A lower temperature tends to enrich the bacterial cultures for mature bacteria because the bacteria are not growing quickly. Hence, at room temperature the culture will have more mature bacteria. The presence or absence of immature bacteria in the culture may influence the rate at which the bacteria are killed, because immature bacteria tend to be more susceptible to many forms of antibiotics.

Cyclic peptides having the following sequences were used for testing.

| | |
|---|---|
| D-Lys-His-D-Leu-Trp-D-Leu-Trp; | (SEQ ID NO: 27) |
| D-Lys-Arg-D-Lys-Trp-D-Leu-Trp-D- | (SEQ ID NO: 19) |
| Leu-Trp; | |
| D-Lys-Gln-D-Arg-Trp-D-Leu-Trp-D- | (SEQ ID NO: 8) |
| Leu-Trp; | |
| D-Lys-Lys-D-Leu-Trp-D-Leu-Trp; | (SEQ ID NO: 26) |
| D-Arg-Arg-D-Leu-Trp-D-Leu-Trp; | (SEQ ID NO: 29) |
| D-Lys-Ser-D-Lys-Trp-D-Leu-Trp-D- | (SEQ ID NO: 12) |
| Leu-Trp; and | |
| D-Arg-Arg-D-Lys-Trp-D-Leu-Trp-D- | (SEQ ID NO: 18) |
| Leu-Trp. | |

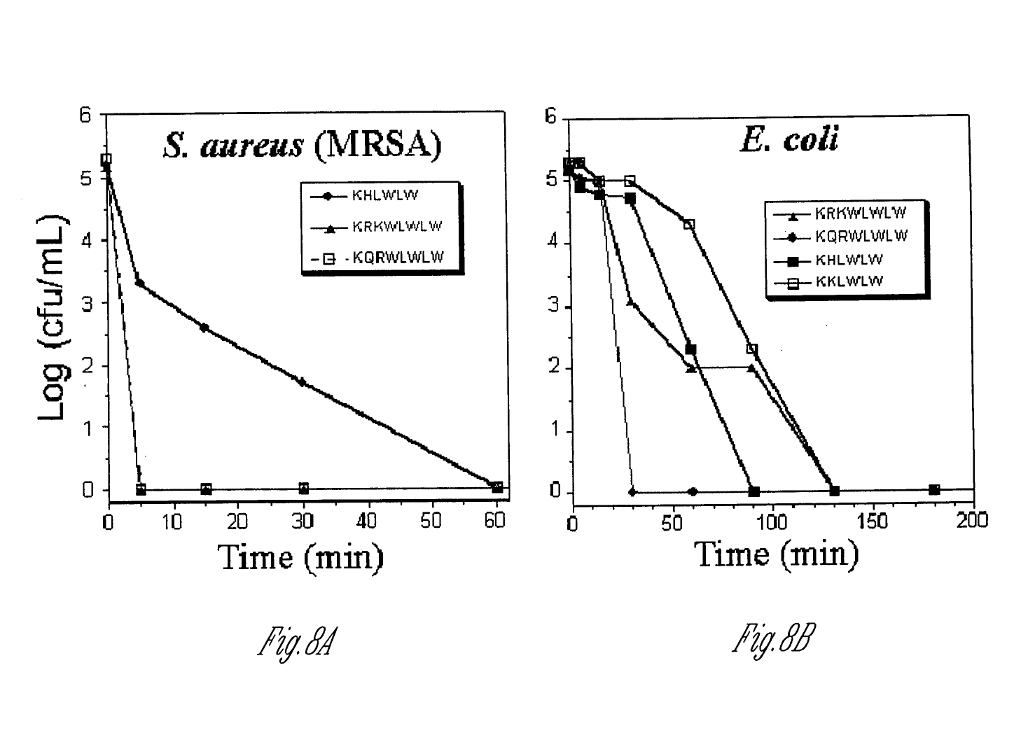

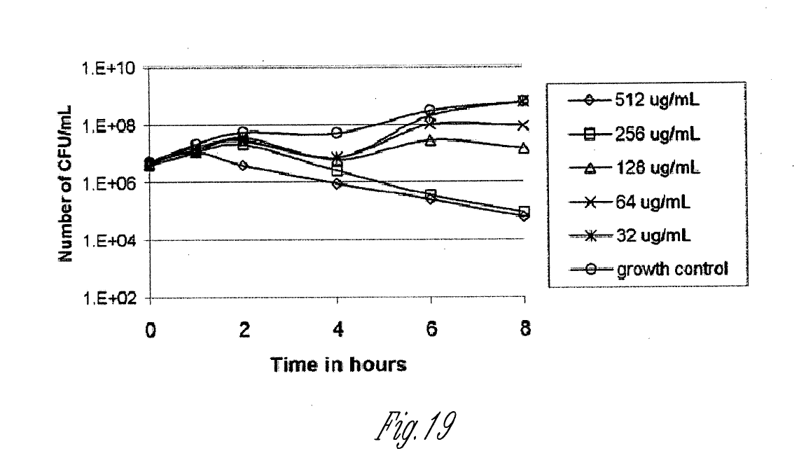

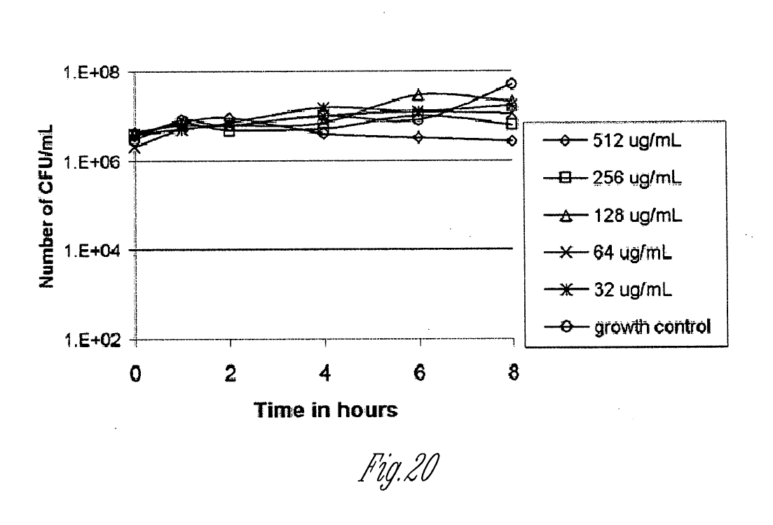

Time curves for killing various types of bacteria are provided in FIGS. 8-20.

To generate the data shown in FIG. 8a, the log of the number of colony forming units (cfu) per ml of culture was determined as a function of time after treatment of S. aureus (MRSA) (ATCC No. 33591) with cyclo[KHLWLW] (closed circles), cyclo[KRKWLWLW] (closed triangles), and cyclo[KQRWLWLW] (open squares) at MIC concentrations. As illustrated in FIG. 8a, cyclic peptides cyclo[KRKWLWLW] and cyclo[KQRWLWLW] killed effectively substantially all or most of the S. aureus (MRSA) within about five minutes. The reduction in cfu count was greater than 3 log units. Cyclic peptide cyclo[KHLWLW] killed effectively all S. aureus (MRSA) within about 60 minutes.

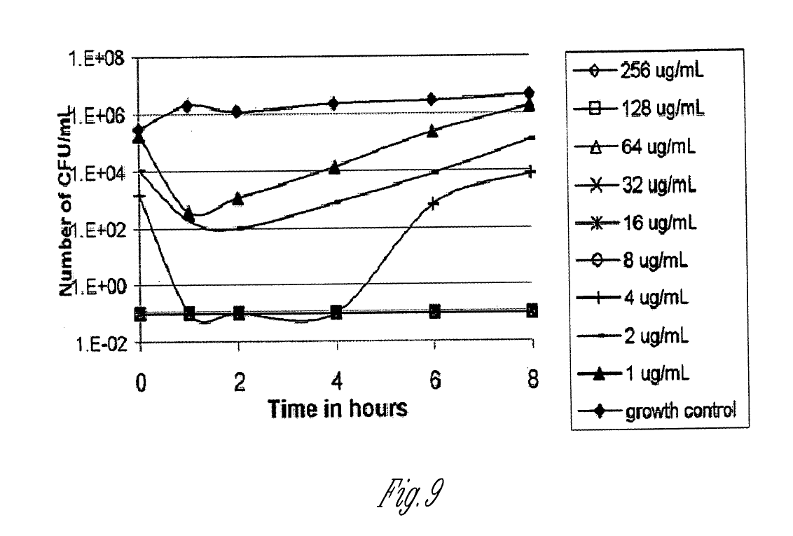

In FIG. 8b, the log of the number of E. coli colony forming units (cfu) per ml of culture is plotted versus time treated with cyclo[KRKWLWLW] (closed triangles), cyclo[KQRWLWLW] (closed circles), cyclo[KHLWLW] (closed squares), and cyclo[KKWLW] (open squares) at MIC concentrations. As illustrated, cyclo[KQRWLWLW] kills effectively all the E. coli within about thirty minutes. Cyclic peptide cyclo[KHKLWLW] kills effectively all E. coli within about 90 minutes. Cyclic peptides cyclo[KRKWLWLW] and cyclo[KKWLW] kill effectively all of the E. coli within about 130 minutes. A comparison of FIGS. 8 and 9 indicates that these cyclic peptides kill gram-positive bacteria like S. aureus (MRSA) (ATCC No. 33592) somewhat faster than they kill gram-negative bacteria like E. coli. Nonetheless, these cyclic peptides kill both types of bacteria very quickly.

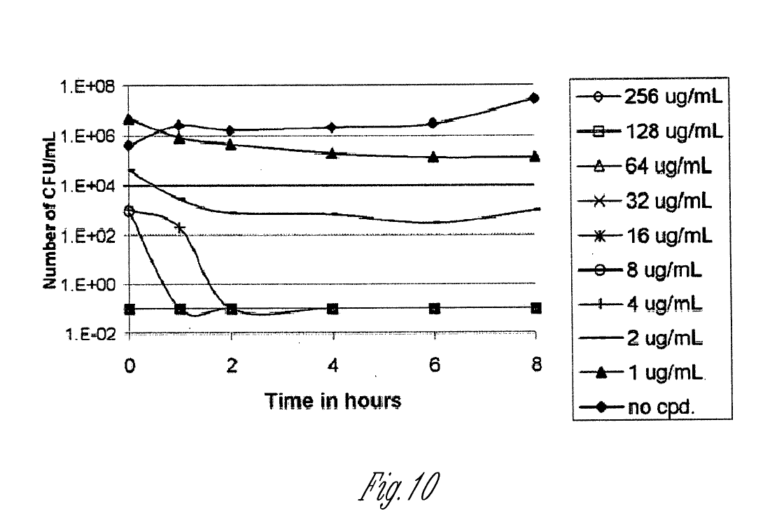

FIGS. 9 and 10 illustrate the time course for killing S. aureus (MRSA) (ATCC No. 33592) with varying concentrations of cyclo[KSKWLWLW] cyclic peptide (SEQ ID NO:12) at room temperature and at 37° C. For example, at 37° C., about 4 μg/ml of the KSKWLWLW cyclic peptide appears to kill effectively all bacteria within about one hour. However, the bacterial population rebounds by about six hours. This may be due to growth of a few survivors after the residual cyclic peptide within the culture has been exhausted because the amount of peptide employed in that assay was below the MIC value. At room temperature, about 4 μg/ml appears to kill all S. aureus (MRSA) by about 2 hours and no rebound is observed. The KSKWLWLW cyclic peptide is highly effective against bacteria at room temperature and at 37° C.

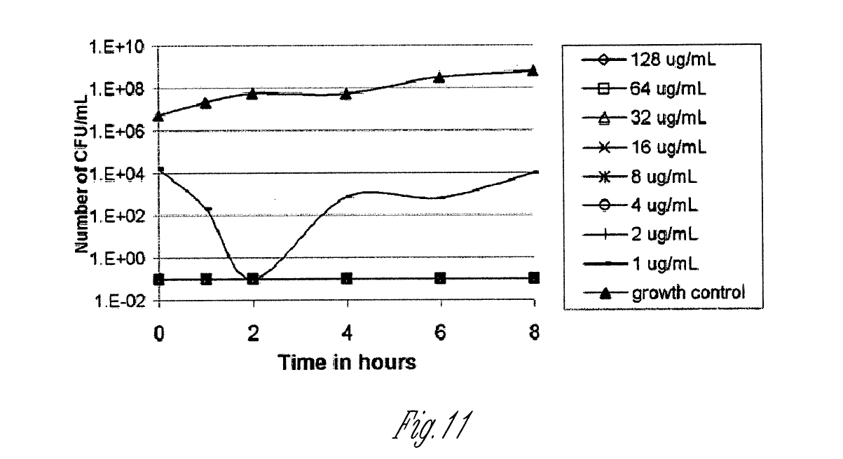

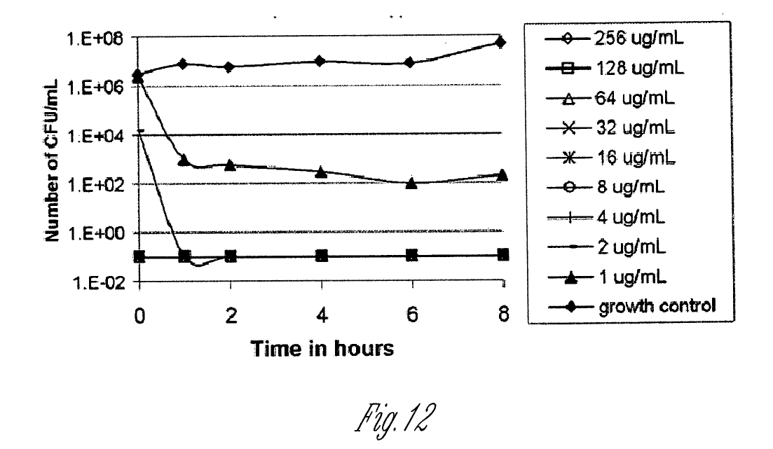

FIGS. 11 and 12 illustrate the time course for killing E. faecium SP 180 with varying concentrations of KSKWLWLW (SEQ ID NO:12), at room temperature and at 37° C. Assays were performed in Brain Heart Infusion broth. As illustrated, somewhat shorter times and/or somewhat lower concentrations of this cyclic peptide are required to kill E. faecium SP180 at 37° C. than at room temperature. E. faecium SP180 is vancomycin resistant (vanA) and multiply resistant to several FDA approved antibiotics. For example, at 37° C., about 2 μg/ml of KSKWLWLW can kill effectively all bacteria faster than an aliquot can be removed from the test culture for assessing the number of surviving cfu. However, at room temperature, discernible numbers of surviving bacteria can be detected immediately after adding about 2 μg/ml of KSKWLWLW. Moreover, when 1 μg/ml of KSKWLWLW is used, effectively no cfu can be detected after 2 hours at 37° C., whereas at room temperature, viable bacteria can be detected throughout the entire time-course. There is a rebound in bacterial viability when using 1 μg/ml of KSKWLWLW at 37° C. This may be due to growth of a few survivors after the residual cyclic peptide within the culture has been exhausted because the amount of peptide employed in that assay was below the MIC value. The KSKWLWLW cyclic peptide is highly effective against E. faecium SP 180 bacteria at both room temperature and at 37° C.

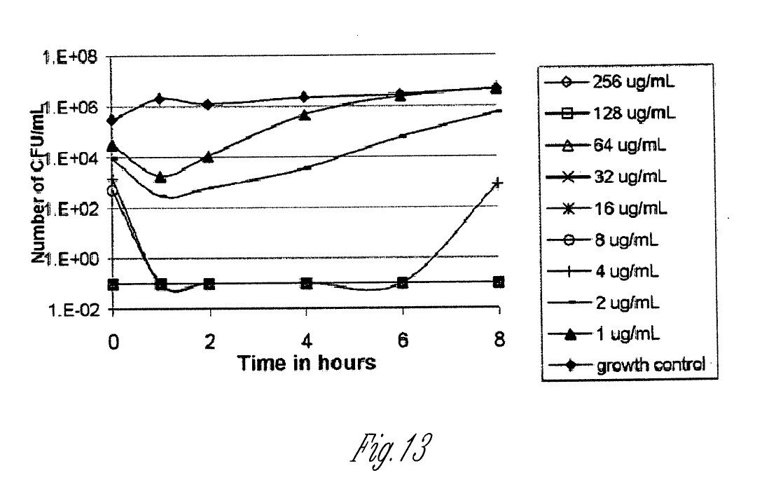

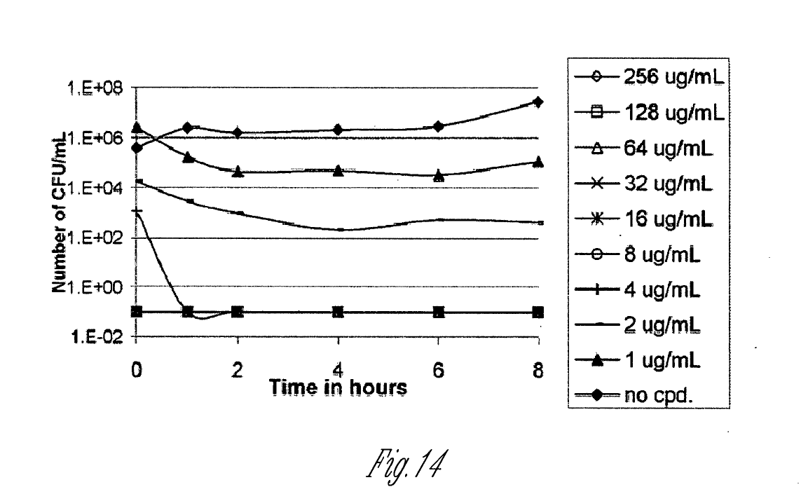

FIGS. 13 and 14 illustrate the time course for killing S. aureus (MRSA) (ATCC No.33592) with varying concentrations of RRKWLWLW (SEQ ID NO:18), at room temperature and at 37° C. For example, at 37° C., about 4 μg/ml of the RRKWLWLW (SEQ ID NO:18) cyclic peptide appears to kill effectively all bacteria within about one hour. However, the bacterial population rebounds by about eight hours. This may be due to growth of a few survivors after the residual cyclic peptide within the culture has been exhausted because the amount of peptide employed in that assay was below the MIC value. At room temperature, about 4 μg/ml appears to kill all S. aureus (MRSA) by about 1 hour after exposure to the cyclic peptide, and no rebound is observed. The RRKWLWLW (SEQ ID NO:18) cyclic peptide is highly effective against bacteria at room temperature and at 37° C.

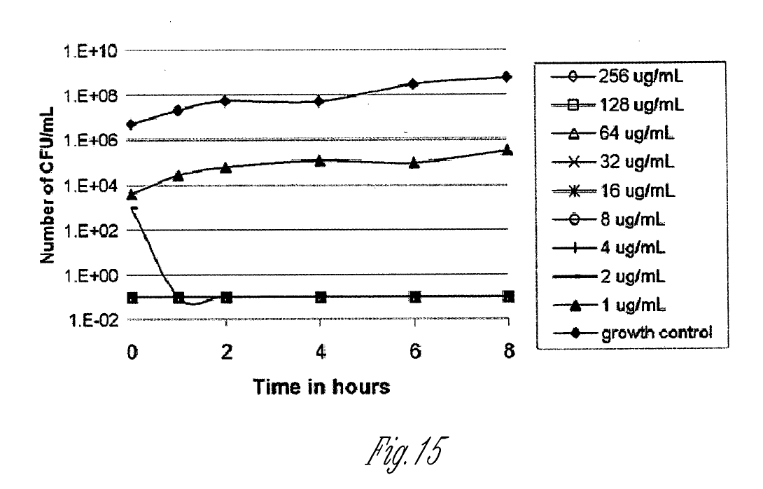

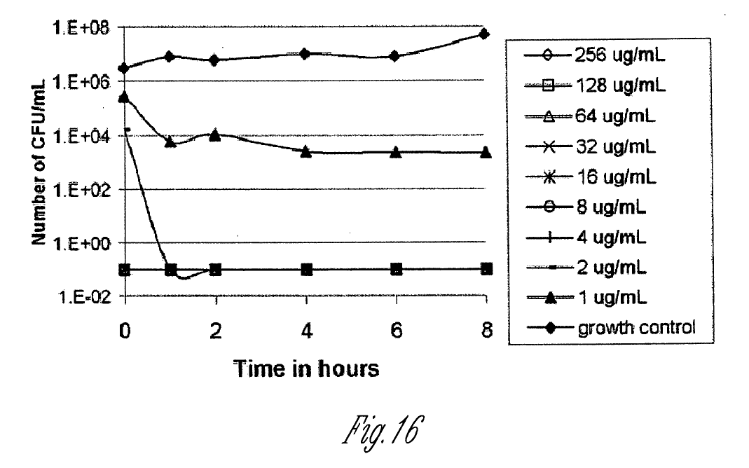

FIGS. 15 and 16 illustrate the time course for killing E. faecium SP180 with varying concentrations of RRKWLWLW (SEQ ID NO:18) at room temperature and at 37° C. E.faecium SP180 is vancomycin resistant (vanA) and multiply resistant to several FDA approved antibiotics. Assays were performed in Brain Heart Infusion broth. As illustrated, somewhat shorter times and/or somewhat lower concentrations of this cyclic peptide are required to kill E. faecium SP180 at 37° C. than at room temperature. For example, at both room temperature and at 37° C., about 2 μg/ml of RRKWLWLW (SEQ ID NO:18) can kill effectively all bacteria within one hour. Hence, the RRKWLWLW (SEQ ID NO:18) cyclic peptide is highly effective against E.faecium SP180 bacteria at both room temperature and at 37° C.

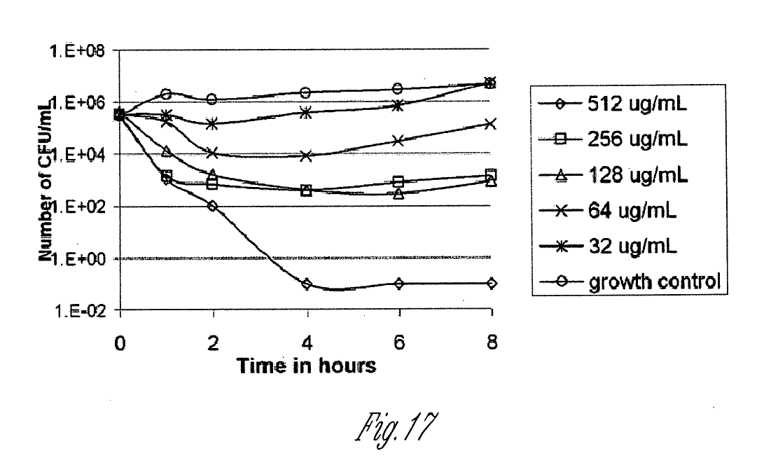

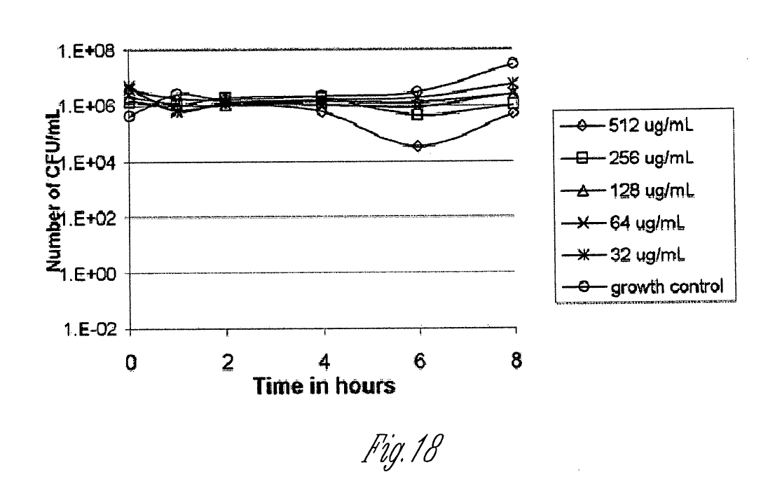

Many conventional antibiotics are merely bacteriostatic, meaning they stop bacteria from growing but do not kill bacteria quickly. This is illustrated in FIGS. 17 and 18, which provide a time course for killing S. aureus (MRSA) (ATCC No. 33592) with varying concentrations of penicillin G. As illustrated, at room temperature no concentration of penicillin G assayed up to 512 μg/ml was effective for killing, or even reducing, the entire population of S. aureus (MRSA) during the entire time course of the study (0-8 hours). However, at the highest concentration employed (512 μg/ml) penicillin G was effective in reducing the number of cfu within four hours at 37° C. In contrast, as shown in FIGS. 8-16, cyclic peptides of the invention quickly kill a variety of mature and immature bacteria at comparatively low concentrations.

### EXAMPLE 3

### Membrane Depolarization Activity

Although not intending to be bound by any mechanism of action, it is believed that one mechanism by which the present cyclic peptides may operate to kill microbes is by membrane depolarization. Another mechanism by which the cyclic peptides may kill microbes is through a receptor that recognizes the cyclic peptides as ligands. See Friederich et al., Antimicrob. Agents Chemother. 44, 2086-2092(2000); Amsterdam, D. in Antibiotics in Laboratory Medicine, 3rd ed. (ed. Lorian, V.) 53-105 (Baltimore, Maryland, USA, 1991). A receptor/ligand-mediated mode of action for the cyclic peptides in bacterial membranes is unlikely for several reasons. First, cyclic peptides with diverse structures are active against microbes, but most receptors discriminate between potential ligands on the basis of structure, and will only recognize ligands having defined structural features. Second, the peptides of the invention kill bacteria very quickly whereas a receptor/ligand-mediated binding/inhibition mechanism would typically be expected to take several hours to achieve complete bactericidal or bacteristatic activity. This example provides biophysical data that supports a membrane depolarization mechanism of action.

The structural diversity of cyclic peptides with activity is illustrated by enantiomeric peptides having SEQ ID NO:8 (cyclo-[D-Lys-Gln-D-Arg-Trp-D-Leu-Trp-D-Leu-Trp]) and SEQ ID NO:9 (cyclo-[Lys-D-Gln-Arg-D-Trp-Leu-D-Trp-Leu-D-Trp]). These two cyclic peptides have similar in vitro activities despite the differences in chirality of these peptides at each position. Moreover, as illustrated by the time-killing studies described in the previous Example, the speed of bacteria killing is not consistent with a receptor/ligand-mediated mode of action. Such a receptor-ligand mechanism would likely require more time to kill bacteria than the short time periods shown in FIGS. 8-16. For example, octameric peptides having SEQ ID NO:8 and SEQ ID NO:19, and hexameric peptides having SEQ ID NO:26 and SEQ ID NO:27 at concentrations equal to or above the MIC have complete bactericidal activity against S. aureus MRSA within 5 and 60 minutes, respectively. Further examples of time-killing studies are provided in the previous Example.

In this Example, cyclic peptides were tested to determine whether they would exhibit cellular depolarization activity. The fluorescence of the cyanine membrane dye 3,3′-dipropylthiadicarbocyanide is sensitive to changes in bacterial membrane potential, and was employed in this Example to follow the effects of peptide SEQ ID NO:8, 18 and 26 on membrane depolarization of live S. aureus (ATCC 25923). See, Sims et al., Biochemistry 13, 3315-3330 (1974); Waggoner, Annu. Rev. Biophys. Bioeng. 8, 847-868 (1979); Loew, Adv. Chem. Ser. 235 (Biomembrane Electrochemistry), 151-173 (1994).

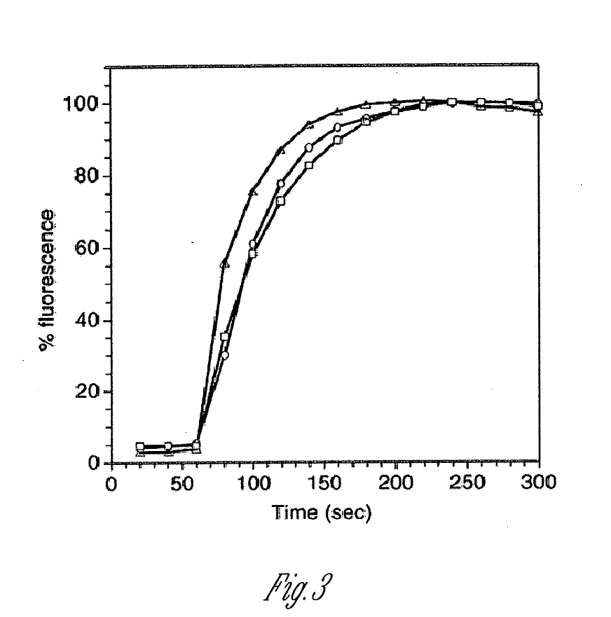

In each case, the exposure of dye-saturated live S. aureus to cyclic peptides at various concentrations (0.1 to 1×MIC, minimum inhibitory concentration) led to fast and complete membrane depolarization (FIG. 3). Culture samples taken during the course of these experiments correlate membrane depolarization with cell death. Substantially no living bacteria could be detected after five minutes exposure to MIC concentrations of peptides, whereas at 0.1×MIC concentrations a significant population of live bacteria was still present.

Biophysical Studies in Synthetic Membranes

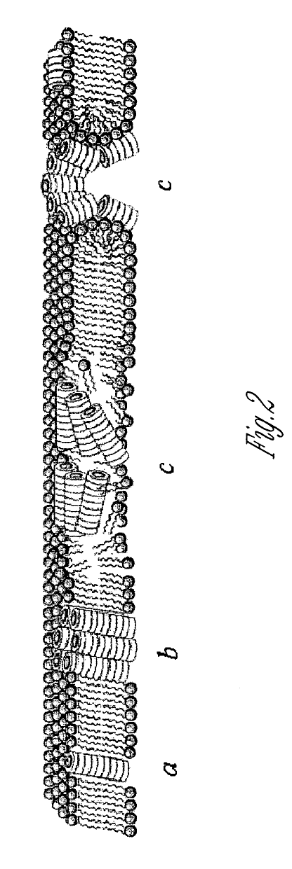

The biophysical analyses performed in synthetic lipid membranes also support a membrane permeation mode of action. Studies support the idea that eight residue cyclic D, L-α-peptides can likely form different types of supramolecular structures depending on the amino acid composition and sequence of the cyclic peptide. For example, the peptide SEQ ID NO:2 (cyclo[-Gln-D-Leu-(Trp-D-Leu)₃]) facilitates only the transport of analytes that are smaller than its internal diameter across liposome membranes and according to ATR-FTIR analysis in DMPC multibilayes, assembles into a tube-like structure that is oriented perpendicular to the membrane plane. Therefore, the single nanotube through-pore mechanism it is a likely mode of function for this peptide (see FIG. 2a).

On the other hand, the homologous peptide SEQ ID NO:3 (cyclo[-Lys-D-Leu-(Trp-D-Leu)₃]), having a polar charged side chain likely forms a different supramolecular structure with an opening that is larger than the internal tube diameter because it facilitates transport of larger molecules of up to approximately 10,000 MW across membranes. Furthermore, the supramolecular structure formed from peptides having SEQ ID NO:3 (cyclo[-Lys-D-Leu-(Trp-D-Leu)₃]) maintains an orientation in synthetic membranes that is perpendicular to the membrane plane according to ATR-FTIR analysis. These data support the conclusion that the peptide likely forms a barrel-stave type supramolecular structure in which multiple upright nanotubes bundle to form a larger pore opening (FIG. 2b).

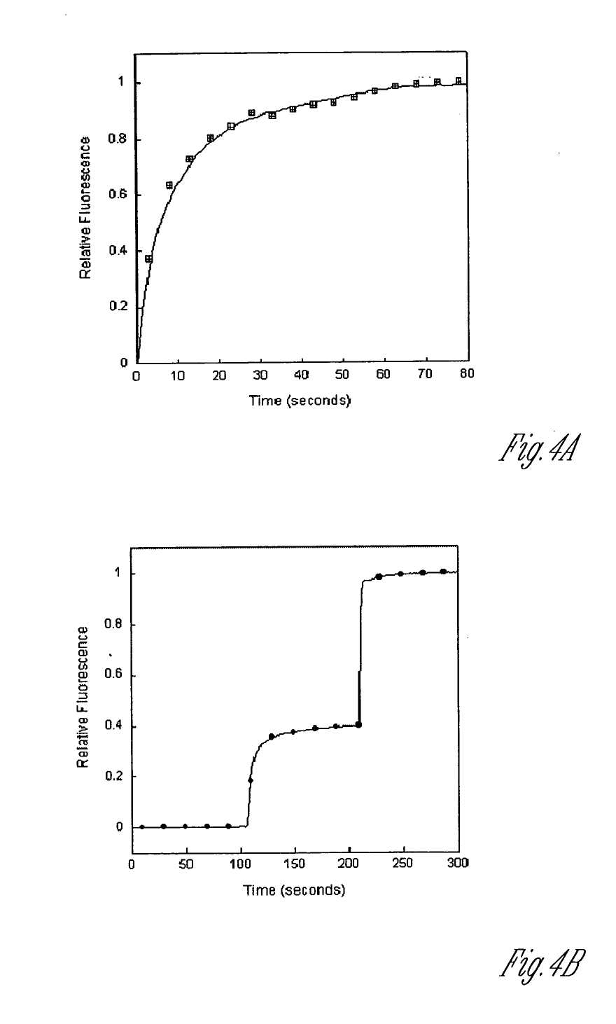

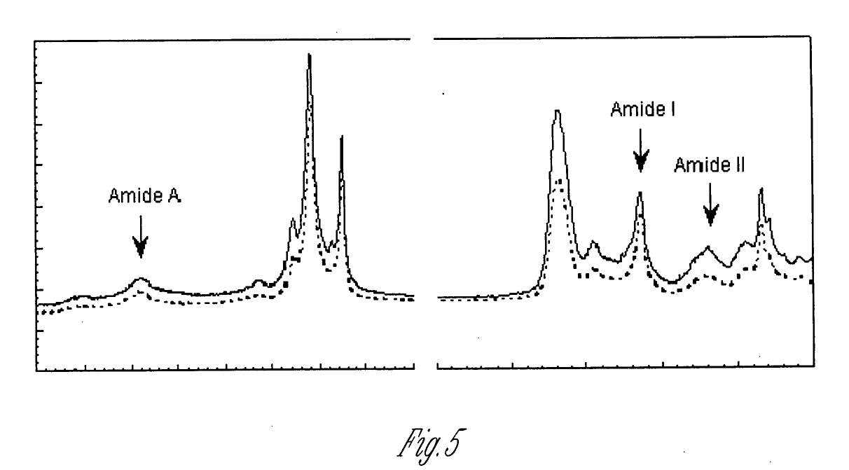

In comparison, peptides like SEQ ID NO:11 (cyclo[-(D-Lys-Gln-D-Lys-(Trp-D-Leu)₂-Trp-], SEQ ID NO:8 (cyclo[-(D-Lys-Gln-D-Arg-(Trp-D-Leu)₂-Trp-], SEQ ID NO:17 (cyclo[-(D-Lys-Lys-D-Lys-(Trp-D-Leu)₂-Trp-], and SEQ ID NO:18 (cyclo[-(D-Arg-Arg-D-Lys-(Trp-D-Leu)₂-Trp-] which possess a hydrophilic face of three contiguous residues, not only mediate the transport of molecules larger than the internal peptide diameter (FIG. 4), but also adopt orientations that are approximately parallel to the plane of the membrane structure. The ATR FT-IR spectroscopy of cyclic peptide SEQ ID NOs:8, 17 and 18 in synthetic lipid membranes reveal amide-I and amide-II bands that are characteristic of tightly hydrogen bonded β-sheet-like structures (Table 11). The observed amide-A (NH stretch) frequencies support a tight network of intersubunit backbone hydrogen bonding. Quantitative measurements in oriented DMPC lipid multibilayers indicate that self-assembled peptide nanotubes are oriented at a 70±5° tilt angle from the membrane normal. These observations suggest a carpet-like mode of membrane permeation (FIG. 2). Increasing the hydrophilicity of the cyclic peptide, thus appears to favor a “surface-seeking” orientation over a “pore-forming” orientation.

TABLE 11

ATR-FTIR data and orientation of antibacterial cyclic
peptide nanotubes in oriented DMPC lipid multibilayers.

| | | | |
|---|---|---|---|
| Cyclic Peptide | Frequency (cm⁻¹) | Peptide | Lipid |

| | | | | | | | | |
|---|---|---|---|---|---|---|---|---|
| SEQ ID NO: | Amide-I | Amide-II | Amide-A | [A∥/A\hat{ }]^{a} | Tilt^{c} | [A∥/A\hat{ }]^{b} | Tilt^{c} | ΔTilt^{d} |

| | | | | | | | | |
|---|---|---|---|---|---|---|---|---|
| 8 | 1629 | 1538 | 3283 | 1.16 | 70° | 1.16 | 29° | 41° |
| [KQRWLWLW] | | | | | | | | |
| 17 | 1629 | 1537 | 3281 | 1.13 | 71° | 1.17 | 29° | 42° |
| [KKKWLWLW] | | | | | | | | |
| 18: | 1628 | 1538 | 3280 | 1.34 | 65° | 1.15 | 29° | 36° |
| [RRKWLWLW] | | | | | | | | |

^{a}The dichroic ratio of the amide-I intensity with parallel polarized incident light to the band intensity with the perpendicular polarized light.
^{b}The dichroic ratio of the antisymmetric CH₂ stretches of DMPC lipid.
^{c}Tilts refer to the angle of the molecular axis with respect to the surface normal.
^{d}Difference between the angles of the peptide tube axis and the lipid hydrocarbon chain. Lipid and peptide nanotube tilt angles are calculated according to methods detailed in H. -S. Kim et al., 120 J. Am. Chem. Soc. 4417-24 (1998). DMPC: dimyristoyl phosphatidylcholine. Data are average of two samples with errors <±2°.

Further evidence for bacterial cellular membrane activity is provided by electron micrographs of bacteria treated with cyclic peptides of the invention. Bacteria (S. aureus, ATCC 25923) were treated with cyclic peptide cyclo[KSKWLWLW] for 120 minutes at room temperature and then prepared for thin section electron microscopy by standard procedures.

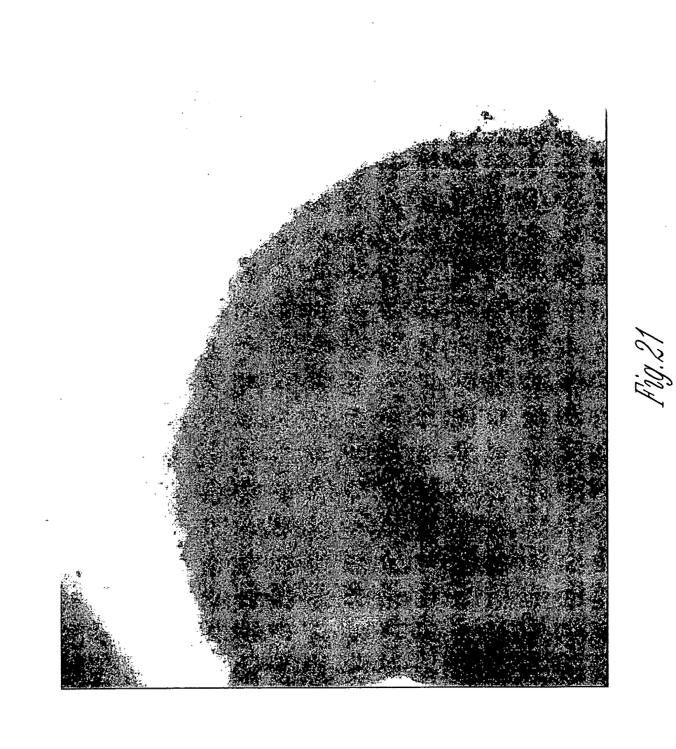

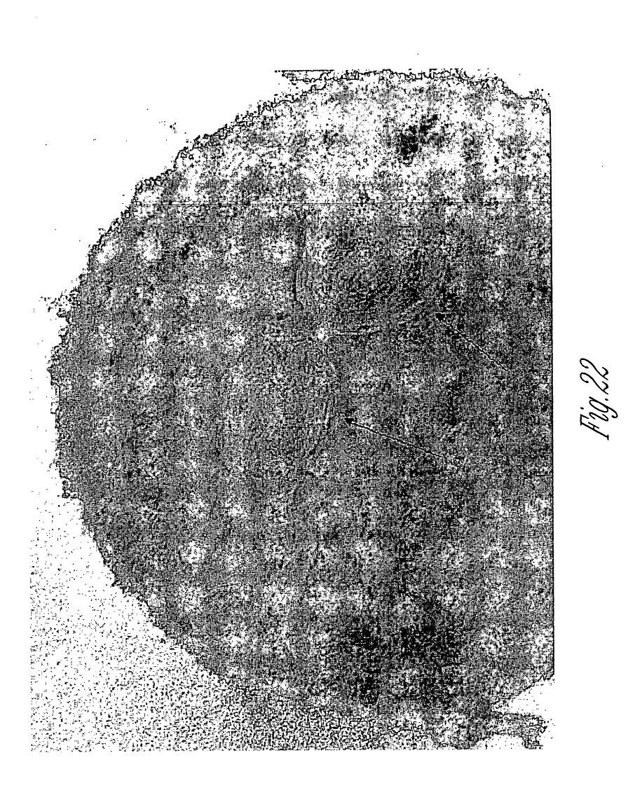

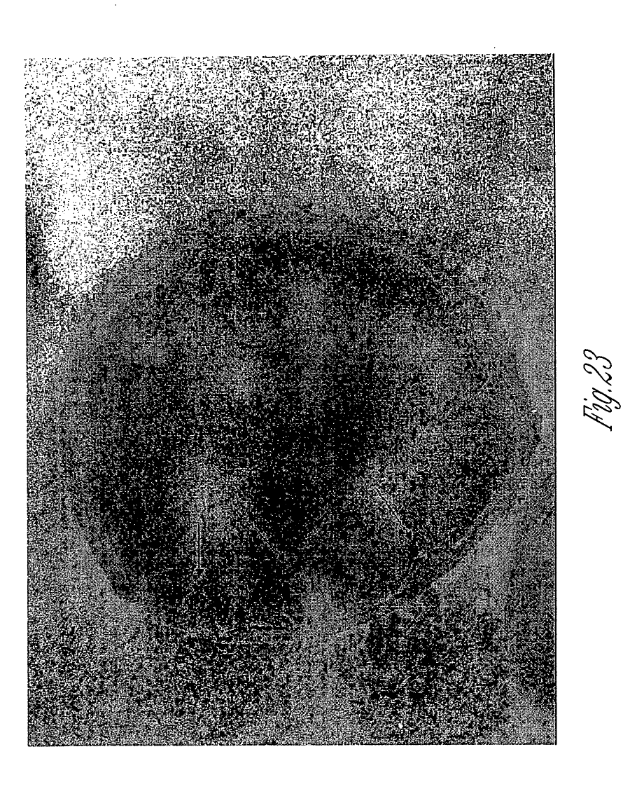

FIGS. 21-23 illustrate the membrane effects caused by cyclic peptides of the invention as observed using the electron microscope. FIG. 21 provides a thin section electron microscopy image of untreated S. aureus (ATCC 25923) displaying a normal intact membrane. FIGS. 22 and 23 provide thin section electron microscopy images of S. aureus (ATCC 25923) after exposure to 2×MIC concentrations of cyclo[KSKWLWLW]. These images provide direct visualization of a membrane mode of action. Arrows denote abnormal membrane structures caused by the peptide action.

Evidence thus indicates an anti-microbial activity based at least in part on membrane permeation, depolarization, and/or lysis. Such evidence includes that the cyclic peptides act very quickly to kill microbes, that diverse cyclic peptide structures described here show anti-microbial activity, that the cyclic peptides can depolarize microbial membranes, that attenuated total reflectance (ATR) FT-IR spectroscopy studies are consistent with a membrane permeation mechanism of action rather than a receptor/ligand-mediated binding/inhibition mechanism, and that electron microscopy reveals an effect of the cyclic peptides of the invention on membrane structure.

### EXAMPLE 4

### Anti-microbial Activity In Vivo

Toxicology Studies: Initial toxicology studies in mice were conducted to evaluate various routes of drug administration, maximum tolerable dose, and blood and tissue toxicity. Two mice in each dose study were injected with a bolus intravenous (IV), intraperitoneal (IP), or subcutaneous (SQ) doses of peptide SEQ ID NO:8, 18, and 26. For each study, two control animals received vehicle only and no peptide. Studies with peptide SEQ ID NO:8 indicate that bolus IV injections of 12 mg/kg cause signs of temporary (<10 min in duration) discomfort. In contrast, no signs of toxicity were observed at the maximum tested dose (12 mg/kg) with the IP route of administration. Peptide SEQ ID NO:26 was tolerated up to the maximum tested IP dose (50 mg/kg), with the highest dose causing signs of acute toxicity that were not observable after one hour. Signs of acute toxicity included any one of the following: lack of activity, red feet and tail, or faster breathing. Peptide SEQ ID NO:18 was tested up to 17.5 mg/kg IP and SQ doses without any apparent signs of toxicity. A regimen of 17.5 mg per day of peptide SEQ ID NO:18 for three consecutive days was administered to two mice IP and two mice SQ. Over four days, no apparent changes in the physical, social, and feeding activities were observed in these mice relative to control mice injected with vehicle without peptide. Moreover, hematology, necropsy and detailed microscopic examination of various tissue and organ samples performed on the fourth day after the initial injection showed normal blood and morphological profiles except for the sites of IP and SQ injections (K. G. Osborn, DVM, Ph.D., the Scripps Research Institute). These sites exhibited moderate subacute inflammation typical of IP and SQ drug administration.

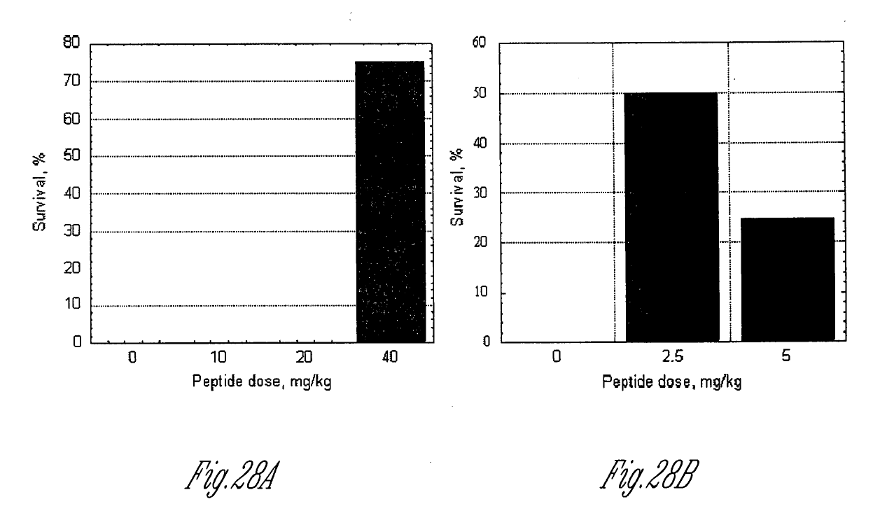

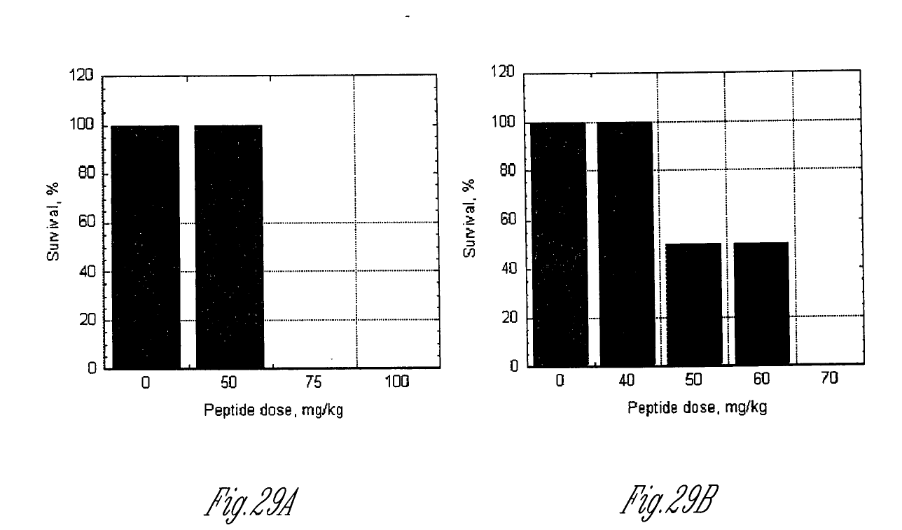

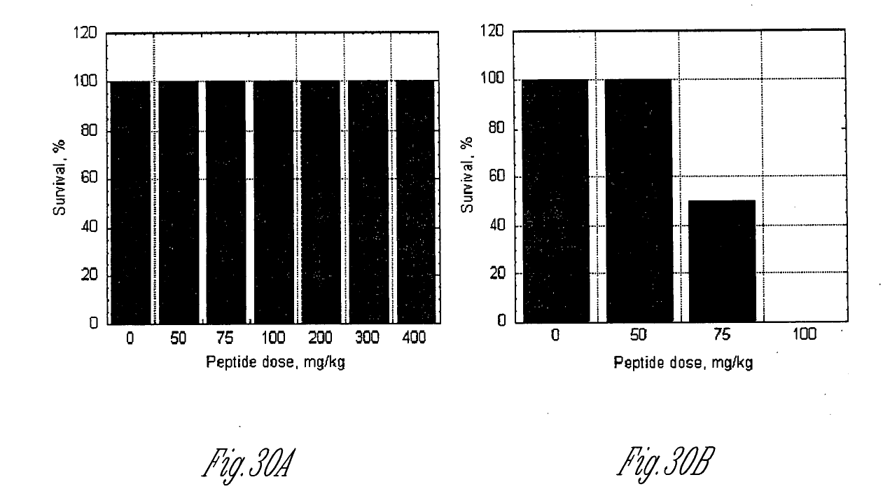

In Vivo Antibacterial Effects: Peptide SEQ ID NO:18 was tested for antibacterial in vivo by observing whether this peptide could protect mice from bacterial infection. Two groups of mice (4 mice per dose in each group) were infected intraperitoneally (left side) with a lethal dose of MRSA (ATCC 33591) (2-5×10⁷ cfu/mouse). To the first group of mice, peptide bolus doses of 0 (vehicle only), 10, 20, and 40 mg/kg were administered subcutaneously (SQ) in the upper neck compartment soon after MRSA injection. To the second group of mice, five bolus peptide doses each 0 (vehicle only), 2.5, and 5 mg/kg were administered intravenously (IV) in 10 hour intervals with the first series of doses administered soon after MRSA injection. All mice in the control groups that received vehicle without peptide (0 mg/kg doses) died within 48 hours. However, at 40 mg/kg bolus SQ dose and 2.5×5 mg/kg IV dosage regimen, 75% and 50% of mice survived, respectively, during the course of a fourteen-day study (FIG. 28).

In general, some variability was observed in the data for SQ and IV treatments. Such variability may result from a low absorption of the peptide administered SQ and/or due to the range of expected experimental errors in IV administration of drugs to mice, a small test animal. Therefore,.we examined the efficacy of intraperitoneal (IP) route of treatment. Six mice were infected intraperitoneally (left side) with a lethal dose of MRSA (2-5×10⁷ cfu/mouse). A 13 mg/kg bolus of the peptide was administered IP (right side) 45 min to an hour after MRSA injection. All mice in the control group that received vehicle without peptide died within 48 hours. However, 67% of mice that received the peptide survived during the course of a seven-day study.

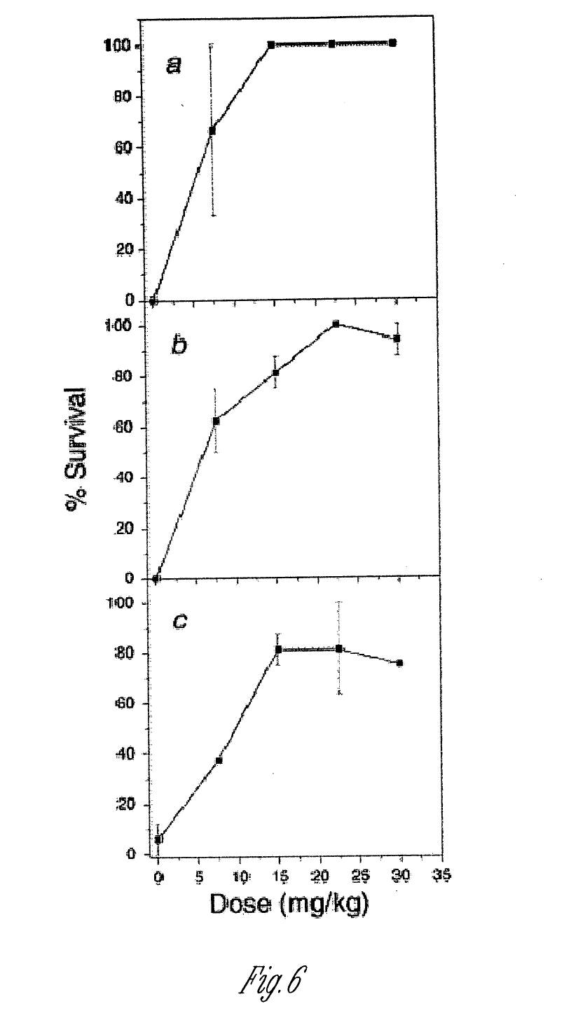

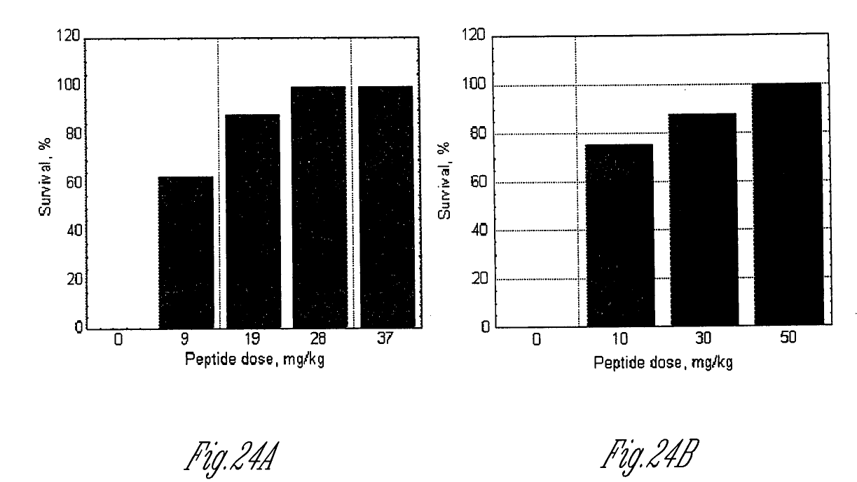

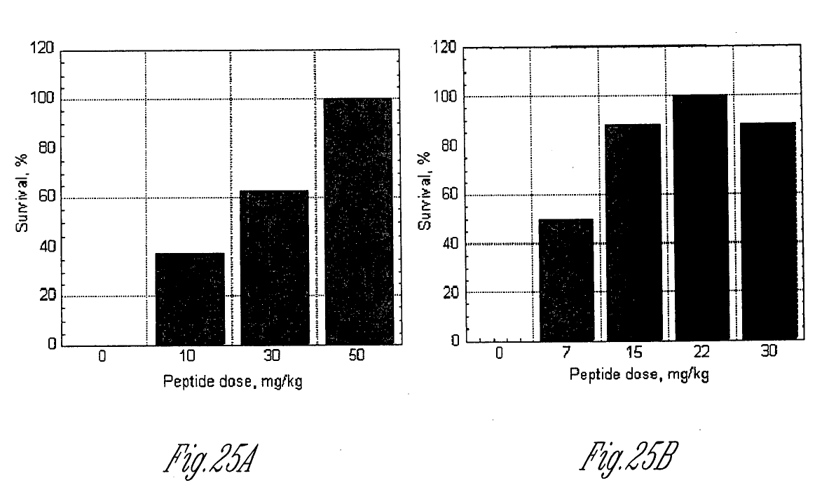

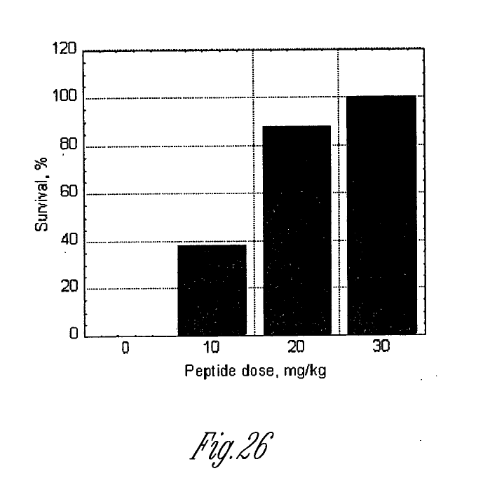

Following this initial demonstration of in vivo antibacterial efficacy, a larger study with peptides having SEQ ID NO:8, 12, 17, 18, and 26 was performed. Groups of mice were infected with lethal doses of MRSA (ATCC 33591) (IP left side). Each group of mice was treated with a bolus EP (right side) dose of peptide SEQ ID NO:8, 12, 17, 18, or 26 at 45-60 min after initial infection. The mice were observed for up to 14 days. All mice in the control group that received vehicle without peptide died within 48 hours. In each case, a single dose of the appropriate amount of peptide was sufficient to completely protect various groups of mice from MRSA infections (FIGS. 6, 24, 25, and 26; Table 12).

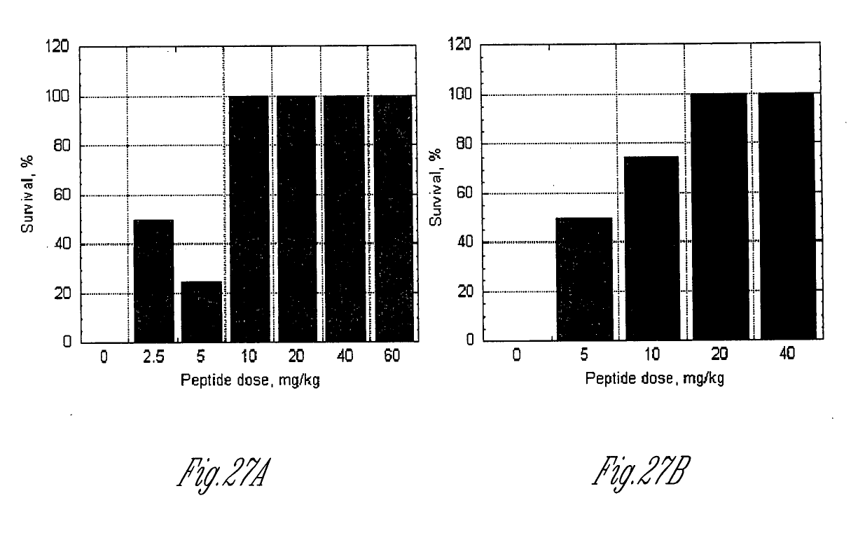

Groups of mice were also infected with lethal doses of VREF (ATCC 51575) (IP left side). Each group of mice was treated with a bolus IP (right side) dose of peptide SEQ ID NO:12, 17, or 18 at 45-60 min after initial infection. The mice were observed for 14 days. All mice in the control group that received vehicle without peptide died within 48 hours. In each case, a single dose of the appropriate amount of peptide was sufficient to completely protect various groups of mice from VFEF infections (FIG. 27, Table 12).

TABLE 12

In vivo Protection Doses (PD₅₀) and
Lethal Doses (LD₅₀) of cyclic peptides

| | | |
|---|---|---|
| PD₅₀ (μg/ml) | LD₅₀(IP) | LD₅₀(IV) |

| | | | | |
|---|---|---|---|---|
| Sequence^{a} | MRSA^{b} | VREF^{c} | (μg/ml) | (μg/ml) |
| c[RRKWLWLW] | 8 | 7 | 60 | 16 |
| (SEQ ID NO: 18) | | | | |
| c[KQRWLWLW] | 7 | — | 55 | >12 |
| (SEQ ID NO: 8) | | | | |
| c[KSKWLWLW] | 20 | 5 | >400 | 70 |
| (SEQ ID NO: 12) | | | | |
| c[KKKWLWLW] | 7 | 13 | — | — |
| (SEQ ID NO: 17) | | | | |
| c[KKLWLW] | 15 | — | 75 | 24 |
| (SEQ ID NO: 26) | | | | |

^{a}Underlined letters represent D-residues.
^{b}MRSA (ATCC 33591).
^{c}VREF (ATCC 51575). Male Balb- C mice were used in groups of 8 (for MRSA) and 4 (for VREF and lethal doses).

Remarkably, the effective doses were parallel to those required to kill bacteria in vitro. Specifically, the dosages of peptides having SEQ ID NO:18, 8, 12, 17, and 26 at which 50% of animals are protected from S. aureus MRSA infection (PD₅₀ ) were 8 mg/kg, 7 mg/kg, 20 mg/kg, 7 mg/kg, and 15 mg/kg, respectively. Similarly, the dosages of peptides having SEQ ID NO:18, 12 and 17 at which 50% of animals are protected from E. faecium VREF infection (PD₅₀) were 7 mg/kg, 5 mg/kg and 13 mg/kg, respectively. The trend of these values is similar to the trend of the MIC values observed for in vitro anti-bacterial studies, as shown by Table 13, below.

TABLE 13

Comparison of Effective Amounts In Vitro and In Vivo

| | |
|---|---|
| In Vitro | In Vivo |

| | MRSA | VREF | RBC | MRSA | VREF | |
|---|---|---|---|---|---|---|
| | MIC | MIC | MIC | PD₅₀ | PD₅₀ | LD₅₀(ip) |
| Peptide | (μg/mL) | (μg/mL) | (μg/mL) | mg/kg | mg/kg | mg/kg |
| c[RRKWLWLW] | 6 | 5 | 50 | 8 | 7 | 60 |
| (SEQ ID NO: 18) | | | | | | |
| c[KQRWLWLW] | 12 | 12 | 40 | 7 | — | 55 |
| (SEQ ID NO: 8) | | | | | | |
| c[KSKWLWLW] | 5 | 15 | 100 | 20 | 5 | >400 |
| (SEQ ID NO: 12) | | | | | | |
| c[KKLWLW] | 10 | 40 | 80 | 15 | — | 75 |
| (SEQ ID NO: 26) | | | | | | |

These in vivo studies support the use of the cyclic peptides of the present invention as anti-microbial agents, including use of agents in the treatment of multiply drug-resistant Staphylococcus aureus, Enterococcus feacium and other bacterial infections.

Pharmacokinetic Studies: Pharmacokinetic parameters for peptides cyclo[RRMWLWLW] and cyclo[KSKWLWLW] in mice and for commercial antibiotics in humans are provided in Table 14.

TABLE 14
Pharmacokinetic Comparisons

| Pharmacokinetic | c[RRKWLWLW] | c[KSKWLWLW] | Vancomycin^{a} | Erythromycin^{a} |
|---|---|---|---|---|
| Parameter | Mouse | Mouse | Human | Human |
| Kₑₗ (1/h) | 0.6 | 0.84 | 0.12 | 0.58 |
| V_{D} (L/kg) | 0.58 | 0.67 | 0.47 | — |
| T_{1/2} (h) | 1.20 | 0.83 | 6 | 1.2 |
| CL (mL/min) | 0.16 | 0.30 | 66 | — |
| F_{IP} (%) | 9 | 32 | — | — |

Kₑₗ - elimination rate constant,
V_{D} - volume of distribution,
T_{1/2} - half life time,
CL - total clearance,
F_{IP} - bioavailability via IP route.
^{a}from David Bourne, Ph.D., www.boomer.org/c/p1/

### EXAMPLE 5

### Cyclic β-Peptides Self-Assemble to Form Transmembrane Ion Channels

The ion transport properties of tetrapeptides cyclo[(β-Trp)₄] and cyclo[(β-Trp-β-Leu)₂], synthesized via established protocols, were studied in liposome-based assays and by single channel conductance measurements in planar lipid membranes. The observed conductance in 500 mM KCl with peptide concentrations of about 30 mM in the subphase is 56 pS, corresponding to the rates of channel mediated K⁺ transport of 1.9×10⁷ ions s⁻¹ for both tetrapeptides. Such a transport speed is more than twice that of gramicidin A under similar conditions.

FT-IR studies in lipid membranes were also undertaken that provided evidence of transmembrane channels formed by these peptides. Peptide preparations displayed all expected peptide IR signals including amide I, amide II and N-H bands. The observed amide N-H stretching bands at 3289 cm⁻¹ and 3297 cm⁻¹ indicate the existence of tight backbone hycdrogen-bonding networks with an average inter-subunit distance of 4.8 Å that are consistent with solid state IR data on cyclic D, L-α-peptides. Therefore, cyclic β-peptides can also form transmembrane ion channels.

All publications and patents are incorporated by reference herein, as though individually incorporated by reference. The invention is not limited to the exact details shown and described, for it should be understood that many variations and modifications may be made while remaining within the spirit and scope of the invention defined by the statements.

1. A pharmaceutical composition for treating or preventing a microbial infection in an animal comprising a pharmaceutically acceptable carrier and a cyclic peptide having a sequence of from four to about sixteen alternating D- and L-α-amino acids in an amount effective to treat or prevent said infection caused by a target microbial organism in said animal.

2. The pharmaceutical composition of claim 1, wherein the cyclic peptide has a minimum inhibitory concentration at which substantially no target microbial organisms grow in vitro that is less than about one half the peptide concentration needed to cause 50% hemolysis of mammalian red blood cells in vitro.

3. The pharmaceutical composition of claim 1, wherein the cyclic peptide has a minimum inhibitory concentration at which substantially no target microbial organisms grow in vitro that is less than one fifth the peptide concentration needed to cause 50% hemolysis of mammalian red blood cells in vitro.

4. The pharmaceutical composition of claim 1, wherein the cyclic peptide has a minimum inhibitory concentration at which substantially no target microbial organisms grow in vitro that is less than one tenth the peptide concentration needed to cause 50% hemolysis of mammalian red blood cells in vitro.

5. The pharmaceutical composition of claim 1, wherein the cyclic peptide has a minimum inhibitory concentration at which substantially no target microbial organisms grow in vitro that is less than one twentieth the peptide concentration needed to cause 50% hemolysis of mammalian red blood cells in vitro.

6. The pharmaceutical composition of claim 1, wherein the cyclic peptide causes substantially no hemolysis of mammalian red blood cells in vitro.

7. The pharmaceutical composition of claim 1, wherein the target microbial organism is a gram-positive bacterium or a gram-negative bacterium.

8. The pharmaceutical composition of claim 1, wherein the cyclic peptide self-assembles into a supramolecular structure that is more disruptive of cellular membranes of the target microbial organism than of animal cell membranes.

9. The pharmaceutical composition of claim 8, wherein the supramolecular structure comprises a nanotube, a barrel of associated, axially parallel nanotubes, a carpet of associated nanotubes, or mixtures thereof.

10. The pharmaceutical composition of claim 8, wherein the supramolecular structure induces depolarization of the target microbial organism selectively over membrane depolarization of animal cells.

11. The pharmaceutical composition of claim 8, wherein the supramolecular structure induces lysis of the target microbial organism selectively over animal cell lysis.

12. The pharmaceutical composition of claim 1, wherein the cyclic peptide comprises a plurality of amino acids having side chains with affinity for biomolecules integral to membranes of the target microbial organism.

13. The pharmaceutical composition of claim 1, wherein the effective amount is an amount of the cyclic peptide sufficient to induce lysis of target microbial organisms without undesired toxicity against animal cells.

14. The pharmaceutical composition of claim 1, wherein the effective amount induces substantially no hemolysis of mammalian red blood cells in vitro.

15. The pharmaceutical composition of claim 1, wherein the effective amount is a single dosage.

16. The pharmaceutical composition of claim 1, wherein the cyclic peptide has at least one polar D- or L-amino acid.

17. The pharmaceutical composition of claim 1, wherein the cyclic peptide has at least two polar D- or L-amino acids.

18. The pharmaceutical composition of claim 1, wherein the cyclic peptide has at least three polar D- or L-amino acids.

19. The pharmaceutical composition of claim 1, wherein the cyclic peptide has four to six polar D- or L-amino acids.

20-29. (canceled)

30. The pharmaceutical composition of claim 1, wherein the cyclic peptide has at least one nonpolar D- or L-amino acid residue.

31. The pharmaceutical composition of claim 1, wherein the cyclic peptide has two to fifteen nonpolar D- or L-amino acid residues.

32. The pharmaceutical composition of claim 30 or 31, wherein each nonpolar D- or L-amino acid residue is alanine, valine, isoleucine, leucine, methionine, norleucine, phenylalanine, tyrosine or tryptophan.

33. A pharmaceutical composition for treating or preventing a microbial infection in an animal comprising a pharmaceutically acceptable carrier and a cyclic peptide having a sequence of about six alternating D- and L-α-amino acids in an amount effective to treat or prevent said infection caused by a target microbial organism in said animal.

34. A pharmaceutical composition for treating or preventing a microbial infection in an animal comprising a pharmaceutically acceptable carrier and a cyclic peptide having a sequence of about eight alternating D- and L-α-amino acids in an amount effective to treat or prevent said infection caused by a target microbial organism in said animal.

35. The pharmaceutical composition of any one of claims 1, 33 or 34, wherein the cyclic peptide has an amino acid sequence of formula I: 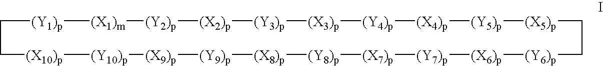
wherein:
m is an integer ranging from 1 to 7;
each p is separately an integer ranging from 0 to 7;
each X₁, X₂, X₃, X₄, X₅, X₆, X₇, X₈, X₉, and X₁₀ is separately a polar D- or L-α-amino acid; and
each Y₁, Y₂, Y₃, Y₄, Y₅, Y₆, Y₇, Y₈, Y₉, and Y₁₀ is separately nonpolar D- or L-α-amino acid; and
wherein the cyclic peptide has an even number of from four to about sixteen alternating D- and L-α amino acids.

36. The pharmaceutical composition of claim 1, 33 or 34, wherein the cyclic peptide has an amino acid sequence of formula II: 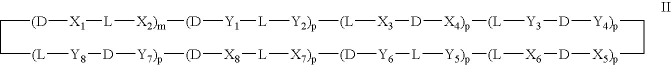
wherein:
m is an integer ranging from 1 to 7;
each p is separately an integer ranging from 0 to 7;
each X₁, X₂, X₃, X₄, X₅, X₆, X₇, and X₈ is separately a polar D- or L-α-amino acid;
each Y₁, Y₂, Y₃, Y₄, Y₅, Y₆, Y₇, and Y₈ is separately nonpolar D- or L-α-amino acid; and
wherein the cyclic peptide has an even number of from four to about sixteen alternating D- and L-α amino acids.

37. The pharmaceutical composition any one of claims 1, 33 or 34, wherein the cyclic peptide has an amino acid sequence having having formula III: 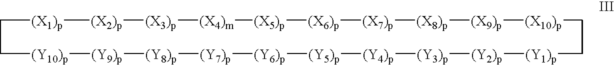
wherein:
m is an integer ranging from 1 to 7;
each p is separately an integer ranging from 0 to 7;
each X₁, X₂, X₃, X₄, X₅, X₆, X₇, X₈, X₉, and X₁₀ is separately a polar D- or L-α-amino acid;
each Y₁, Y₂, Y₃, Y₄, Y₅, Y₆, Y₇, Y₈, Y₉, and Y₁₀ is separately nonpolar D- or L-α-amino acid; and
wherein the cyclic peptide has an even number of from four to about sixteen alternating D- and L-α amino acids.

38. The pharmaceutical composition any one of claims 1, 33 or 34, wherein the cyclic peptide has an amino acid sequence of formula IVa or IVb: 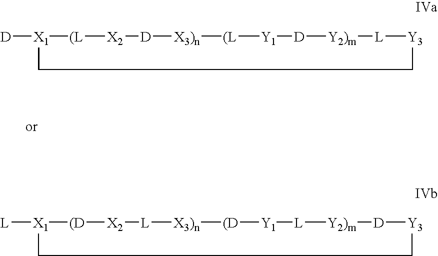
wherein:
n is an integer ranging from 0 to 4;
m is an integer ranging from 1 to 7;
X₁, X₂ and X₃ are each a separate a polar amino acid;
Y₁, Y₂ and Y₃ are each a separate nonpolar amino acid; and
wherein the cyclic peptide has an even number of from four to about sixteen alternating D- and L-α amino acids.

39. The pharmaceutical composition of claim 1, 33 or 34, wherein the cyclic peptide has an amino acid sequence of formula Va or Va: 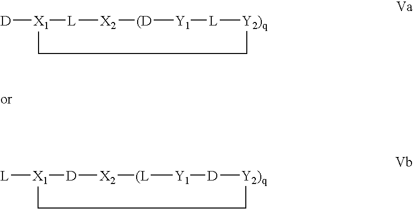
wherein:
q is an integer ranging from 2 to 7;
X₁ and X₂ are separately polar amino acids;
Y₁ and Y₂ are separately nonpolar amino acids.

40. The composition of claim 1, wherein the composition comprises a mixture of two or more different cyclic peptides.

41. The pharmaceutical composition of claim 1, wherein the amino acid sequence comprises any one of SEQ ID NO:8, 9, 12, 17, 18, 26, 29, 47-52, 61, 63, 67, 68, 72-77, 84, 85, 87-89, 91-93, 100, 102, 107, 111,112, 119, 125 or 139.

42-133. (canceled).

134-160. (canceled)

161. A peptide comprising a cyclic amino acid sequence of about six alternating d- and l-α-amino acids, said peptide having anti-microbial activity but substantially no anti-animal cell activity.

162-171. (canceled)

172-177. (canceled)

178-180. (canceled)

181. A pharmaceutical composition for treating or preventing a microbial infection in an animal comprising a pharmaceutically acceptable carrier and a cyclic peptide having a sequence of eight alternating d- and l-α-amino acids in an amount effective to treat or prevent said infection caused by a target microbial organism in said animal, wherein said cyclic peptide comprises the contiguous amino acid sequence K-W-L-W-K.

182. The pharmaceutical composition of claim 181, wherein said cyclic peptide comprises the contiguous amino acid sequence K-W-L-W-K-X1-X2-X3, wherein X1, X2, and X3 are independently selected from the amino acids K, S, Q, and H.

183. The pharmaceutical composition of claim 181 wherein said cyclic peptide is selected from SEQ ID NO:85, SEQ ID NO:87, SEQ ID NO:88, SEQ ID NO:91, SEQ ID NO:92, SEQ ID NO:93, SEQ ID NO:119, and SEQ ID NO:125.
